(19) United States
(12) Patent Application Publication
(10) Pub. No.: US 2019/0185852 A1
(43) Pub. Date: Jun. 20, 2019

(54) THERAPEUTICALLY MODULATING APOB AND APOAI

(71) Applicants: M. Mahmood HUSSAIN, Brooklyn, NY (US); Liye ZHOU, Brooklyn, NY (US); The Research Foundation for the State University of New York, Albany, NY (US)

(72) Inventors: M. Mahmood HUSSAIN, Brooklyn, NY (US); Liye ZHOU, Brooklyn, NY (US)

(21) Appl. No.: 16/099,095

(22) PCT Filed: May 5, 2017
(86) PCT No.: PCT/US2017/031240

§ 371 Date: Nov. 5, 2018

Related U.S. Application Data
(60) Provisional application No. 62/332,442, filed on May 5, 2016.

(51) Int. Cl. C12N 15/113; A61P 9/10; A61P 3/06

(52) U.S. Cl. CPC C12N 15/113; A61P 9/10; A61P 3/06; C12N 2310/14; C12N 2320/35; C12N 2320/32; C12N 2310/141

(57) ABSTRACT

MicroRNAs can be used to decrease expression of apolipoprotein B (apoB), increase expression of apolipoprotein A (apoA), and decrease expression of NCOR1. Use of these microRNAs can simultaneously reduce LDL and increase HDL in circulation and have applications in prevention and treatment of atherosclerosis, hyperlipidemia, and cardiovascular disease as well as other disorders associated with high apoB and/or low apoAI levels.

### CROSS REFERENCE TO RELATED APPLICATIONS

This application claims priority to U.S. Provisional Application No. 62/332,442, entitled “THERAPEUTICALLY MODULATING APOB AND APOAI,” filed May 5, 2016, the disclosure of which is hereby incorporated by reference in its entirety.

### GOVERNMENT LICENSE RIGHTS

This invention was made with government support under grant numbers 2R56DK046900-17A1 and 5R01DK46900-15 from the National Institutes of Health. The government has certain rights in the invention.

### FIELD

The subject technology generally relates to methods of altering the expression of proteins involved in lipid transport and metabolism, for example, to prevent and treat cardiovascular diseases and risk factors such as atherosclerosis and hyperlipidemia.

### INCORPORATION BY REFERENCE

All publications, patents, and patent applications cited herein are incorporated by reference in their entirety. U.S. application Ser. No. 14/370,846, filed Jan. 9, 2013, also relates to methods of treating atherosclerosis and hyperlipidemia with a microRNA, the content of which is hereby incorporated by reference in its entirety.

### BACKGROUND

High plasma concentrations of plasma low density lipoprotein (LDL) and low plasma concentrations of high density lipoprotein (HDL) cholesterol levels are risk factors for cardiovascular diseases. Thus, an ideal treatment goal is to simultaneously decrease LDL and increase HDL.

### SUMMARY OF THE INVENTIONS

The subject technology provides methods of administering a microRNA (miR) comprising SEQ ID NO:1, wherein the miR simultaneously reduces plasma LDL, increases plasma HDL, and enhances hepatic fatty acid oxidation (FAO) and reverse cholesterol transport. In some embodiments, the methods of the subject technology reduce hepatic very low density lipoprotein (VLDL) production.

In some embodiments of the subject technology, the miR further comprises a sequence with at least 70%, 75%, 80%, 85%, 90% or 95% identity to SEQ ID NO:2. In another embodiment, the miR is hsa-miR-1200 (Dharmacon) (referred to herein as “miR-1200”), and has the sequence of SEQ ID NO:2. See Table 1.

TABLE 1
Seed and Full Sequences of miR-1200

| MiR | Seed Sequence | Full Sequence |
|---|---|---|
| Hsa-miR-1200 | SEQ ID NO: 1: | SEQ ID NO: 2: |
| (″miR-1200″) | UCCUGA | CUCCUGAGCCAUUCUGAGCCUC |

In some aspects of the subject technology, a miR comprising SEQ ID NO:1 is administered to a mammal. In some embodiments, the mammal is a mouse. In yet another embodiment, the mammal is an Apoe^{-/-} mouse. In some embodiments, the mammal is a human. In yet another embodiment, the methods of the subject technology provide for the administration of a therapeutically effective amount of a miR comprising SEQ ID NO:1 to a human in need thereof, wherein the treatment prevents or reduces hyperlipidemia or atherosclerosis.

In some embodiments of the subject technology, a therapeutically effective amount of miR comprising SEQ ID NO:1 for treatment of a human is 0.1-2 mg/kg/week. In some of these embodiments, the therapeutically effective amount is 0.1-0.5 mg/kg/week, 0.5-1 mg/kg/week, 1-1.5 mg/kg/week, 1.5-2 mg/kg/week, 0.1 mg/kg/week, 1 mg/kg/week, 1.5 mg/kg/week or 2 mg/kg/week. A person of ordinary skill in the art would understand that this initial dose can be adjusted based on the severity and type of condition being treated, the mode of administration and the response of the individual patient. The dose may also be administered twice a week as a divided dose, biweekly, or as an extended release formulation.

In some embodiments of the subject technology, apoAI expression is increased by contacting a cell with an inhibitor of BCL11B. In one aspect of the subject technology, a miR comprising SEQ ID NO:1 increases apoAI transcription by reducing the expression and/or activity of its repressor, BCL11B. In another aspect of the subject technology, a miR comprising SEQ ID NO:1 reduces apoB expression by targeting the 3′-untranslated region of mRNA and enhancing posttranscriptional degradation. In yet another aspect of the subject technology, a miR comprising SEQ ID NO:1 increases hepatic fatty acid oxidation by repressing NCOR1.

In some embodiments of the subject technology, apoAI expression is increased by contacting a cell with an inhibitor of NRIP1. The inhibitor may be a nucleic acid inhibitor, such as an siRNA, or it may be a small molecule, peptide or protein inhibitor, such as an antibody or a fusion protein. Inhibitors of NRIP1 may be administered in combination with another inhibitor, such as an inhibitor of BCL11B or apoB expression. In one aspect of the subject technology, an NRIP1 inhibitor is administered to an animal or human in an amount sufficient to increase apoAI expression, thereby causing a therapeutically desirable effect, such as preventing or treating atherosclerosis and/or hyperlipidemia.

In some of the methods of the subject technology, a miR comprising SEQ ID NO:1 is administered to prevent, mitigate or reduce atherosclerosis, hyperlipidemia, dyslipidemia, cardiovascular disease. In other methods of the subject technology, a miR comprising SEQ ID NO:1 is administered to prevent, mitigate or reduce insulin resistance, type II diabetes, schizophrenia, fatty liver disease, inflammation, hepatitis C, familial hypercholesterolemia, multiple sclerosis and rheumatoid arthritis.

The subject technology provides methods of reducing plasma LDL and increasing plasma HDL without causing liver injury. In one aspect provided herein, miR-1200 significantly reduced plasma LDL- and increased HDL-cholesterol in diet-induced hyperlipidemic mice. In another embodiment, an miR comprising SEQ ID NO:1 reduces plasma LDL and increases plasma HDL in a hyperlipidemic human.

### BRIEF DESCRIPTION OF THE DRAWINGS

FIGS. 1A-1D show the identification of miRs regulating apoB and apoAI secretion in Huh-7 cells. (A) Huh-7 cells were reverse transfected in duplicate plates with a human miRDIAN mimic 16.0 library (Dharmacon) of 1237 miRs. After 24 hours, cells received complete media with 10% FBS. After another 24 hours, cells were incubated with complete media containing 10% fetal bovine serum (FBS) and oleic acid/BSA complexes (0.4 mM/1.5%) for 2 hours to avoid identification of miRs that affect posttranslational degradation of apoB. Media apoB and apoAI were quantified by ELISA. As a control, different wells in each plate were transfected with Scr (negative) or miR-30c (positive, reduces apoB). (B) Percent change in media apoB and apoAI in two plates exposed to the same miRs compared to Scr control were plotted. (C) The number of miRs that changed media apoB and apoAI to different extents in the second screening were tabulated. (D) Different miR family members with the same seed sequence showed similar effects on media apoB and apoAI.

FIGS. 2A-2J show regulation of apoB secretion by miR-1200 in human hepatoma cells. (A) Reverse transfection of miR-1200 [50 nM] in Huh-7 cells significantly increased miR-1200 levels after 48 h. (B, C) Dose-dependent effects of miR-1200 and anti-1200 on media (B) and cellular (C) apoB. (D, E) Temporal changes in media (D) and cellular (E) apoB levels after treatment with 50 nM of miR-1200, anti-1200 or Scr control. (F) The effect of miR-1200 and anti-1200 on apoB mRNA levels normalized to Scr. (G) Time dependent disappearance of apoB mRNA in cells transfected with miR-1200 and treated with actinomycin D (1 μg/mL). The apoB/18S rRNA ratio at time 0 was set to 100%. (H) Top: Predicted interactions between miR-1200 and apoB mRNA from miRanda. Bottom: The seed interacting site was mutated as indicated, using the primers shown in Table 3 (I) Cells were first transfected with psiCHECK luciferase constructs (1.5 μg/well) containing normal (WT) or mutated (Mut) apoB 3′-UTR sequences. Equal numbers of these cells were transferred to another plate and reverse transfected with either Scr or miR-1200 [50 nM]. The ratios of firefly and Renilla luciferase activities determined after 48 h are shown relative to Scr. (J) Proposed working model: miR-1200 targets the 3′-UTR of apoB, induces mRNA degradation, and decreases the production of apoB-containing lipoproteins. Data are represented as mean±SD. p<0.05, ** P<0.01 and *** P<0.001.

FIGS. 3A-3J show that MiR-1200 increases apoAI secretion by reducing expression of BCL11B, a repressor. (A) Dose-dependent effect of miR-1200 and anti-1200 on apoAI in Huh-7 cells measured after 48 h. (B) Time-dependent changes in media apoAI levels in Huh-7 cells transfected with 50 nM miR-1200, anti-1200 or Scr control. (C) Effect of miR-1200 and anti-1200 on mRNA levels of apoAI normalized to Scr. (D) Temporal changes in apoAI mRNA levels in cells transfected with Scr or miR-1200 and treated with actinomycin D (1 μg/mL). (E) Luciferase activity in Huh-7 cells transfected first with a vector in which human apoAI promoter was cloned upstream of Gaussia luciferase cDNA and then transfected with either miR-1200 or Scr. The luciferase activities were determined after 48 h. Data are presented relative to Scr. (F) MiR-1200 or different siRNAs [50 nM] were reverse transfected in Huh-7 cells. apoAI (left) and apoB (right) were measured in the media after 48 h. (G) Huh-7 cells were co-transfected with different miRs and siRNAs [50 nM]. Secreted apoAI and apoB levels were measured after 48 h. (H) BCL11B mRNA levels were quantified in Scr control, miR-1200, or siBCL11B (SEQ ID NO:4, Table 4) transfected Huh-7 cells. (I) Cells were first transfected with a plasmid expressing luciferase under the control of apoAI promoter and then transfected with miR-1200 or Scr. The luciferase activities were determined after 48 h. (J) Proposed working model: Under normal conditions, BCL11B binds to the apoAI promoter to repress transcription. In miR-1200 overexpressing cells, miR-1200 decreases mRNA levels of BCL11B leading to de-repression of apoAI transcription and increases in mRNA levels. Data are represented as mean±SD. p<0.05, ** P<0.01 and *** P<0.001.

FIGS. 4A-4H show that MiR-1200 differentially regulates HDL and non-HDL cholesterol levels in diet induced hyperlipidemic mice. Male C57BL/6 mice were fed a Western diet for 6 weeks and injected retro-orbitally with miR-1200 or PBS (n=5). Plasma samples were collected 4 days after each injection. (A) A schematic diagram showing amounts of miR injected (top) and times of blood collected (bottom). (B) miR-1200 levels were quantified in different tissues of miR-1200 injected mice and normalized to levels in the small intestine (SI) where the lowest amounts were found. (C) Injection of miR-1200 did not change the expression levels of another endogenous miR, miR-30c, compared to PBS group. (D) Hepatic mRNA levels of different target and non-target genes were quantified in two groups of mice. (E-F) Temporal changes in indicated plasma constituents. (G) Western blot analysis of plasma apolipoproteins and their quantifications by ImageJ. (H) Plasma samples from each group were pooled and subjected to FPLC. Distribution of lipids in different lipoproteins is shown. Data are represented as mean±SD. p<0.05, ** P<0.01 and *** P<0.001. Data are representative of 3 independent experiments.

FIGS. 5A-5H show that MiR-1200 enhances fatty acid oxidation. (A) Hepatic cholesterol and triglyceride were measured in liver homogenates from FIG. 4. (B) Liver slices from FIG. 4 were used to measure fatty acid oxidation and syntheses of fatty acids, triglycerides and phospholipids. (C) Gene expression changes in the livers of mice injected with miR-1200 and PBS. (D) Predicted interaction sites of miR-1200 in the 3′-UTRs of human and mouse NCOR1 mRNA. (E) Huh-7 cells were transfected with 50 nM of miR-1200 or Scr control. After 48 hours, FAO and syntheses of lipids were measured. (F) The mRNA levels of NCOR1 in miR-1200 transfected Huh-7 cells. (G) Huh-7 cells were co-transfected with indicated different siRNA and miRs (50 nM each) to test their effects on fatty acid oxidation. (H) Proposed working model: Under normal conditions, NCOR1 interacts with PPARa/RXR heterodimer (PPARa) to reduce the expression of genes involved in FAO. In miR-1200 overexpressing cells, NCOR1 expression will be reduced resulting in its dissociation from PPARa and allowing the binding of PGC1a to increase the expression of genes involved in FAO. Data are represented as mean±SD. p<0.05, ** P<0.01 and *** P<0.001.

FIGS. 6A-6E show that MiR-1200 decreases VLDL production and promotes reverse cholesterol transport. Male C57B1/J mice were fed on a Western diet for 6 weeks and then injected with 1 mg/kg/week of miR-1200 or PBS (n=7). (A) Time course of plasma lipid levels. (B) Four days after the third injection, mice were divided into two groups. In one group, mice were fasted for 18 hours and injected with Poloxamer 407 and [³⁵S] Promix to study VLDL production (n=3). Time dependent changes in plasma triglyceride were measured. (C) apoB was immunoprecipitated from plasma samples obtained from 2 hour time points and visualized by autoradiography (left). apoB bands were quantified with ImageJ (right). Amounts of newly secreted apoAI were too low to detect. (D) Mice in the second group were intraperitoneally injected with ³H-cholesterol labeled and Ac-LDL loaded J774.1A macrophages (n=4). Radioactivity was measured in plasma, feces, and livers after 48 hours to assess reverse cholesterol transport (RCT). (E) J774A.1 macrophages were loaded with ³H-cholesterol and Ac-LDL and used for 6 h cholesterol efflux studies. Left: Plasma samples (5%) from PBS or miR-1200 treated C57BL/6J mice from FIG. 4 were used for cholesterol efflux (n=5). Right: Isolated HDL (5%) was used as cholesterol acceptor. Data are represented as mean±SD. p<0.05, ** P<0.01 and *** P<0.001.

FIGS. 7A-7H show that MiR-1200 reduces plasma cholesterol and atherosclerosis in Apoe^{-/-} mice. Western diet fed male Apoe^{-/-} mice were injected with 2 mg/kg/week of miR-1200 or PBS (n=5). (A) Quantification of miR-1200 in different organs and hepatic miR-30c levels. (B) Hepatic expression levels of target and non-target genes. (C) Temporal changes in total plasma cholesterol, phospholipid, and triglyceride. (D) Plasma samples from each group were pooled and fractionated by FPLC. Cholesterol, phospholipid and triglyceride were measured in each fraction. The inserts show amplified HDL peaks. (E) Plasma AST, ALT, and CK activities were measured at the end. (F) Livers from two groups were used for hepatic lipids quantification. (G) Aortic arches were exposed, photographed and quantified. (H) Aortas were isolated, fixed and stained with Oil Red O. ImageJ was used for quantification of the atherosclerotic lesions. Data are represented as mean±SD. p<0.05, ** P<0.01 and *** P<0.001.

FIG. 8 provides a graphical summary of miR-1200 regulation

FIG. 9 shows that Hsa-miR-1200 is present in the intron of ELMO1 and is conserved in primates. The top line shows schematic representation of different introns and exons in the human ELMO1 gene. MiR-1200 resides in intron 6 of the gene. Pre-miR-1200 sequences are highly conserved in primates and are highlighted with gray after alignment using Clustal W.

FIG. 10 shows: (top) predicted base-pairing at four different sites between miR-1200 and the 3′-UTR of human BCL11B; (bottom) three miR-1200 target sites on BCL11B 3′-UTR that are well conserved in different species. MiRanda was used to predict potential targets of miR-1200.

FIGS. 11A-11C show that (A-B) MiR-1200 regulates apoB and apoAI in HepG2 cells. Human hepatoma HepG2 cells were reverse transfected with miRs [50 nM]. NT: non-transfected. Media and cellular apolipoproteins were measured after 48 hours. (C) Effect of miR-1200 on mRNA levels in mouse hepatoma AML12 cells. AML 12 cells were plated and were forward transfected with miR-1200 and Scr control [50 nM] using Lipofectamine RNAiMAX. Expression levels of indicated genes were quantified after 48 hours.

FIGS. 12A-12E show that miR-1200 reduces plasma cholesterol and atherosclerosis in Apoe−/− mice without causing liver injury. Male Apoe−/− mice were fed a Western diet for 6 weeks and then injected with 1 mg/kg/week of miR-1200 or PBS control (n=3). Plasma samples were collected four days after each injection. (A) Hepatic expression levels of target and non-target genes. (B) Hepatic cholesterol and triglyceride levels were measured. (C) Time course of total plasma cholesterol. (D) Time course of changes in plasma triglyceride, ALT, AST, and CK activities. (E) Aortas were isolated, fixed and stained with Oil Red 0. Image J was used to quantify the lesion size.

### DETAILED DESCRIPTION

Despite significant advances in lowering risk factors, cardiovascular diseases (CVD) accounted for 30.8% of deaths in 2003-2013 in the United States, and the estimated annual cost of CVD and stroke for 2011-2012 was about $316.6 billion. Most of the risk factors for CVD are controllable, especially plasma cholesterol, which is carried in the blood by apolipoprotein B (apoB)-containing lipoproteins, such as low-density lipoproteins (LDLs), and non-apoB-containing high-density lipoproteins (HDLs). apoB-containing lipoproteins are primarily synthesized and secreted by the liver and small intestine to transport lipids to other peripheral tissues. Excess accumulation of these lipoproteins and their modifications in the plasma contribute to atherosclerosis as these modified lipoproteins are taken up by macrophages. apoAI interacts with ATP-binding cassette transporter family A and protein 1 (ABCA1) present on the plasma membrane of different cells, especially macrophages, extracts cholesterol and transports it back to the liver for excretion from the body. This reverse cholesterol transport (RCT) is believed to be anti-atherogenic. For these reasons, elevated LDL and low HDL are two well-established risk factors for atherosclerosis.

Statins lower plasma LDL-cholesterol by reducing hepatic cholesterol synthesis and increasing LDL clearance. However, these drugs only decrease the incidence of cholesterol related diseases by 30-40%, and almost 20% of the population fails to respond to or cannot tolerate statins. Further, high doses of statins sometimes cause muscle pain, elevations in plasma levels of liver and muscle enzymes, and new onset of diabetes mellitus.

While PCSK9 inhibitors have been shown to lower plasma cholesterol, PCSK9 inhibitors have also been associated with neurocognitive side effects. Because the target of both statins and PCSK9 inhibitors is the LDL receptor, these drug classes are not useful in the treatment of homozygous familial hypercholesterolemia subjects that are deficient in this receptor. Prior to the subject technology, no effective therapeutic methods were available to increase functional HDL to prevent CVD. Thus, a need remains for novel therapeutic agents that modulate plasma LDL and HDL to achieve therapeutically beneficial outcomes.

Other known methods for reducing LDL include total plasma exchange (TPE) and LDL apheresis. TPE replaces all plasma every 7-14 days and can reduce plasma LDL to below target levels. HDL levels are also severely reduced however, and the sharp decrease in LDL is followed by a rebound phase as new VLDL is synthesized and secreted. LDL apheresis is similar in that it selectively removes apoB containing lipoproteins, but unlike TPE, LDL apheresis spares HDL. The side effects for both procedures, however, include hypotension, anemia, and hypocalcaemia. Moreover, these treatments are time consuming, invasive and not universally available.

In severe cases, liver transplantation may also be a viable option to lower lipid levels and prevent early onset cardiac events. Liver transplantation is however costly, not readily available globally, and limited by the availability of suitable donors.

MicroRNAs (miRs) are small (˜22 nucleotides) non-coding RNAs that target multiple genes and affect multiple pathways by interacting with the 3′-untranslated region (3′-UTR) of mRNA and destabilizing mRNA or blocking translation. In >70% of cases, miRs mediate regulation by mRNA degradation. MiRs bind to the target mRNA via seed and supplementary sequences. A seed sequence (2-7 nucleotides from the 5′-end of the miR) forms perfect complementary base pairs, while the supplementary site in the 3′-region may or may not form perfect base pairs with the target mRNA. MiRs with the same seed sequence belong to the same family. MiR-30c and miR-33 have been identified to decrease LDL and HDL, respectively, and MiR-148a consistently decreased HDL but had variable effects on plasma LDL levels. However, no MiR has previously been shown to both decrease LDL and increase HDL.

High plasma LDL and low HDL cholesterol levels are risk factors for cardiovascular diseases. Although therapeutics would ideally both lower LDL and increase HDL, there were no known drug therapies that concomitantly mitigate these risk factors prior to the subject technology. Moreover, existing therapeutics such as statins and PCSK9 inhibitors are only partially effective and can cause serious adverse effects.

The subject technology provides methods of administering a miR comprising SEQ ID NO:1, wherein the miR decreases apoB and increases apoAI in a mammal, resulting in lower levels of LDL and higher levels of HDL in plasma.

In some embodiments of the subject technology, the miR comprises a sequence with at least 70%, 75%, 80%, 85%, 90% or 95% identity to SEQ ID NO:2. In certain embodiments, the miR is miR-1200, and has the sequence of SEQ ID NO:2. (See Table 1.)

The subject technology provides methods of simultaneously lowering plasma LDL and increasing plasma HDL. In some embodiments of the subject technology, a microRNA comprising SEQ ID NO:1 is administered to a mammal, wherein the microRNA reduces plasma LDL and increases plasma HDL via different mechanisms, thus mitigating dyslipidemia and atherosclerosis. In some embodiments, the microRNA is miR-1200.

The subject technology includes methods of significantly reducing apoB (an LDL structural protein) while increasing apoAI (main HDL protein) secretion. In some embodiment, the methods reduce apoB while increasing apoAI in cell culture. In some embodiments, the methods reduce apoB while increasing apoAI in hepatic or hepatoma cells. In some embodiments, the methods reduce apoB while increasing apoAI in the liver of a human or other mammal. In some aspects of the subject technology, apoB expression is decreased by an inhibitor that causes degradation of mRNA encoding apoB, e.g. the human apoB mRNA (Gene accession NM_000384, Appendix A). In other aspects of the subject technology, apoAI expression is increased by an inhibitor that causes degradation of mRNA encoding a repressor of ApoAI, such as NRIP1, e.g. human NRIP1 mRNA (Gene accession NM_003489, Appendix A) and/or BCL11B, e.g. human BCL11B mRNA (Gene accession NM_022898, Appendix A).

In some embodiments of the subject technology, apoAI is increased by inhibiting its repressor, BCL11B. In some embodiments, BCL11B expression is inhibited by a miR. In yet another embodiment, BCL11B is inhibited by an RNA longer than 20 nucleotides, such as an RNA that is longer than 30, 50, 75, 100, 125 or 200 nucleotides. In another embodiment, BCL11B is inhibited by a nucleic acid comprising modified nucleotides, a double-stranded nucleic acid inhibitor, a protein inhibitor or a small molecule inhibitor.

In some embodiments of the subject technology, apoAI is increased by inhibiting its transcriptional repressor, NRIP1. In some embodiments, NRIP1 expression is inhibited by an siRNA. In yet another embodiment, NRIP1 is inhibited by an RNA longer than 20 nucleotides, such as an RNA that is longer than 30, 50, 75, 100, 125 or 200 nucleotides. In another embodiment, NRIP1 is inhibited by a nucleic acid comprising modified nucleotides, a double-stranded nucleic acid inhibitor, a protein inhibitor or by a small molecule inhibitor. In some embodiments, NRIP1 inhibitors are administered to an animal or human, alone or in combination with inhibitors of BCL11B and/or apoB, to achieve a therapeutically effective result, such as treating or preventing hyperlipidemia and/or atherosclerosis.

A microRNA is a short RNA. MicroRNAs may also be denoted miRNA or miR herein. Preferably a miRNA to be used with the subject technology is 19-25 nucleotides in length and consists of non-protein-coding RNA. Mature miRNAs may exert, together with the RNA-induced silencing complex, a regulatory effect on protein synthesis at the post-transcriptional level. More than 1500 human miRNA sequences have been discovered to date and their names and sequences are available from the miRBase database (http://www.mirbase.org).

A miRNA of the subject technology can be synthesized, altered, or removed from the natural state using a number of standard techniques known in the art. A synthetic miRNA, or a miRNA partially or completely separated from its coexisting materials is considered isolated. An isolated miRNA can exist in substantially purified form, or can exist in a cell into which the miRNA has been delivered. A miRNA can be chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Rosetta Genomics (North Brunswick, N.J.), Pierce Chemical (part of Perbio Science, Rockford, Ill., USA), Glen Research (Sterling, Va., USA), ChemGenes (Ashland, Mass., USA), Ambion (Foster City, Calif., USA), and Cruachem (Glasgow, UK).

In some embodiments, the miRs of the invention are delivered to target cells using an expression vector encoding the miR. A variety of suitable vectors are known in the art, including plasmids, viruses, and linear polynucleotides. Plasmids suitable for expressing any of the miRs of the subject technology, methods for inserting nucleic acid sequences into the plasmid to express the miR of interest, and methods of delivering the recombinant plasmid to cells of interest are well established and practiced in the art. Examples of suitable plasmids and methods of expression and delivery can be found in Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

In other embodiments, the miRs of the subject technology are expressed from recombinant viral vectors. Non-limiting examples of viral vectors include retroviral vectors, adenoviral vectors (AV), adeno-associated virus vectors (AAV), herpes virus vectors, and the like. Recombinant viral vectors suitable for expressing miRs of the subject technology, methods for inserting nucleic acid sequences for expressing RNA in the vector, methods of delivering the viral vector to cells of interest, and recovery of the expressed RNA molecules are within the skill in the art. Examples include Dornburg (1995), Gene Therap. 2:301-310; Eglitis (1988), Biotechniques 6:608-614; Miller (1990), Hum. Gene Therap. 1:5-14; and Anderson (1998), Nature 392:25-30, the entire disclosures of which are herein incorporated by reference.

Various modifications to the miRs of the subject technology can be introduced as a means of increasing intracellular stability, therapeutic efficacy, and shelf life. Some modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

In yet other embodiments, the miRs of the subject technology are expressed from recombinant circular or linear plasmids using any suitable promoter. Selection of suitable promoters is within the skill in the art. Suitable promoters include but are not limited to U6 or H1 RNA pol III promoter sequences or cytomegalovirus promoters. Recombinant plasmids can also comprise inducible or regulatable promoters for miRNA expression in cells. For example, the CMV intermediate-early promoter may be used with the miRNAs of the subject technology to initiate transcription of the miRNA gene product coding sequences.

A further embodiment of the subject technology provides a method of preventing or treating a disease associated with high apoB and/or low apoAI levels, including but not limited to insulin resistance, type II diabetes, schizophrenia, fatty liver disease, inflammation, hepatitis C, familial hypercholesterolemia, and rheumatoid arthritis.

An additional embodiment of the subject technology provides a method of preventing or treating a disease associated with reduced LDL and increased HDL, including but not limited to cardiovascular disease (coronary artery disease, peripheral arterial disease, cerebral vascular disease, cardiomyopathy, hypertensive heart disease, cardiac dysrhythmias, inflammatory heart disease, aortic aneurysm, renal artery stenosis, valvular heart disease), atherosclerosis, fatty liver disease, diabetic dyslipidemia, and hypocholesterolemia.

In one embodiment, the subject technology features changing levels of apoB, apoAI, HDL, and/or LDL with a microRNA administered with additional agents at a therapeutically effective amount. The term “therapeutically effective amount,” as used herein, refers to the total amount of microRNA and each additional agent that is sufficient to show a meaningful benefit to the subject.

Pharmaceutical compositions of the subject technology can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or calcium or sodium salts (for example, calcium chloride, calcium ascorbate, calcium gluconate or calcium lactate).

Delivery of the compositions in the claimed methods may be facilitated by use of a biocompatible gel, a lipid-based delivery system, such as liposomes, polycationic liposome-hyaluronic acid (LPH) nanoparticles (Medina, 2004), LPH nanoparticle conjugated to a peptide, such as an integrin-binding peptide (Liu, 2011), cationic polyurethanes such as polyurethane-short branch-polyethylenimine (PU-PEI), a glycoprotein-disulfide linked nanocarrier (Chiou, 2012) or other known miR delivery systems including, but not limited to dendrimers, poly(lactide-co-glycolide) (PLGA) particles, protamine, naturally occurring polymers, (e.g. chitosan, protamine, atelocollagen), peptides derived from protein translocation domains, inorganic particles, such as gold particles, silica-based nanoparticles, or magnetic particles. (Zhang, 2013).

If desired, the miRs of the subject technology may be modified to protect against degradation, improve half-life, or to otherwise improve efficacy. Suitable modifications are described, e.g. in U.S. Patent Publication Nos. 20070213292, 20060287260, 20060035254, 20060008822, and 20050288244, each of which is hereby incorporated by reference in its entirety.

Pharmaceutical compositions of the subject technology can be packaged for use in liquid or solid form, or can be lyophilized. Conventional nontoxic solid pharmaceutically-acceptable carriers can be used for solid pharmaceutical compositions of the subject technology. Examples of carriers include but are not limited to pharmaceutical grades of mannitol, lactose, starch, magnesium stearate, sodium saccharin, talcum, cellulose, glucose, sucrose, and magnesium carbonate.

Pharmaceutical formulations may be adapted for administration by any appropriate route. For example, appropriate routes may include oral, nasal, topical (including buccal, sublingual, or transdermal), or parenteral (including subcutaneous, intrasternal, intracutaneous, intramuscular, intraarticular, intraperitoneal, intrasynovial, intrathecal, intralesional, intravenous, intradermal injections or infusions). For human administration, the formulations preferably meet sterility, pyrogenicity, general safety, and purity standards, as required by the offices of the Food and Drug Administration (FDA).

The therapeutically effective amount of microRNA varies depending on several factors, such as the condition being treated, the severity of the condition, the time of administration, the duration of treatment, the age, gender, weight, and condition of the subject. In some embodiments of the subject technology, a therapeutically effective amount of miR comprising SEQ ID NO:1 for treatment of a human is 0.1-2 mg/kg/week. In some of these embodiments, the therapeutically effective amount is 0.1-0.5 mg/kg/week, 0.5-1 mg/kg/week, 1-1.5 mg/kg/week, 1.5-2 mg/kg/week, 0.1 mg/kg/week, 1 mg/kg/week, 1.5 mg/kg/week or 2 mg/kg/week. A person of ordinary skill in the art would understand that this initial dose can be adjusted based on the severity and type of condition being treated, the mode of administration and the response of the individual patient. One of ordinary skill in the art may also modify the route of administration in order to obtain the maximal therapeutic effect. Where a dosage regimen comprises multiple administrations, the effective amount of the miRNA molecule administered to the subject can comprise the total amount of gene product administered over the entire dosage regimen.

The microRNA in the subject technology can be administered with additional agents in combination therapy, either jointly or separately, or by combining the microRNA and additional agents(s) into one composition.

For example, the miRNA pharmaceutical compositions of the subject technology can be used to treat hypercholesterolemia or atherosclerosis, either alone or in combination with a statin. Examples of statins include Atorvastatin (Lipitor), Ezetimibe/Simvastatin (Vytorin), Lovastatin (Mevacor), Simvastatin (Zocor), Pravastatin (Pravachol), Fluvastatin (Lescol), and Rosuvastatin (Crestor), Fenofibrate (Lipofen), Gemfibrozol (Lopid) and/or Ezetimibe (Zetia).

In other embodiments, the pharmaceutical compositions of the subject technology are administered in combination with ACE inhibitors, aldosterone inhibitors, angiotensin II receptor blockers (ARBs), beta-blockers, calcium channel blockers, cholesterol lowering drugs, digoxin, diuretics, inotropic therapy, potassium or magnesium, PCSK9 inhibitors (otherwise known as monoclonal antibodies), vasodilators, or warfarin.

Examples of ACE inhibitors include but are not limited to Accupril (quinapril), Aceon (perindopril), Altace (ramipril), Capoten (captopril), Lotensin (benazepril), Mavik (trandolapril), Monopril (fosinopril), Prinivil, Zestril (lisinopril), Univasc (moexipril), and Vasotec (enalapril).

Examples of aldosterone inhibitors include but are not limited to eplernone (Inspra) and spironolactone (Aldoctone).

Examples of angiotensin II receptor blockers (ARBs) include but are not limited to candesartan (Atacand), eprosartan (Teventen), irbesartan (Avapro), Iosartan (Cozar), telmisartan (Micardis), valsartan (Diovan), and olmesartan (Benicar).

Examples of beta-blockers include acebutolol hydrochloride (Sectral), atenolol (Tenormin), betaxolol hydrochloride (Kerlone), bisoprolol fumarate (Zebeta), carteolol hydrochloride (Cartrol), esmolol hydrochloride (Brevibloc), metoprolol (Lopressor, Toprol XL), and penbutolol sulfate (Levatol).

Examples of calcium channel blockers include Amlodipine (Norvasc), Diltiazem (Cardizem, Tiazac), Felodipine, Isradipine, Nicardipine (Cardene SR), Nifedipine (Procardia) Nisoldipine (Sular), and Verapamil (Calan, Verelan, Covera-HS).

The practice of aspects of the subject technology can employ, unless otherwise indicated, conventional techniques of cell biology, cell culture, molecular biology, transgenic biology, microbiology, recombinant DNA, and immunology, which are within the skill of the art. These techniques are fully explained in literature. Examples of conventional techniques can be found in Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986). All patents, patent applications and references cited herein are incorporated in their entirety by reference.

### EXAMPLES

The following specific examples are to be construed as merely illustrative, and not limiting the remainder of the disclosure in any way. It is believed that one skilled in the art can, based on the description herein, utilize the subject technology to its fullest extent.

### Example 1

### Identification of MicroRNAs Regulating apoB and apoAI Secretion from Human Hepatoma Cells

To identify miRs regulating apoB and apoAI secretion, human hepatoma Huh-7 cells were transfected with 1237 human miRs (human miRIDIAN Mimic 16.0 library, Dharmacon).

MiRs were suspended in RNase free water to obtain 2 μM stocks and 3 μL of each miR was added in duplicate wells to obtain a final concentration of 50 nM. 7 μl of Opti-MEM and 10 μl of lipofectamine RNAiMAX (Life technologies) diluted 1:20 in Serum Reduced Opti-MEM was added to each well. After 20 to 30 minutes, 25,000 cells in 100 μl of Opti-MEM were added to each well. After additional 24 hours, culture media were changed with fresh DMEM containing 10% fetal bovine serum. Media were changed 24 hours later and cells were incubated with DMEM containing oleic acid/BSA complex ((oleic acid (0.4 mM)/BSA (1.5%)) for 2 hours.

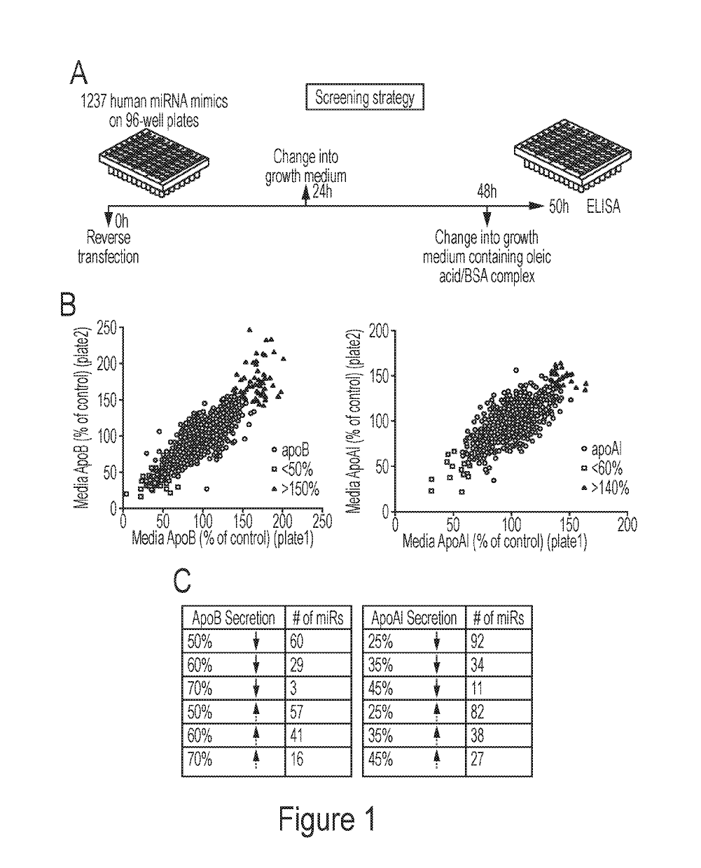

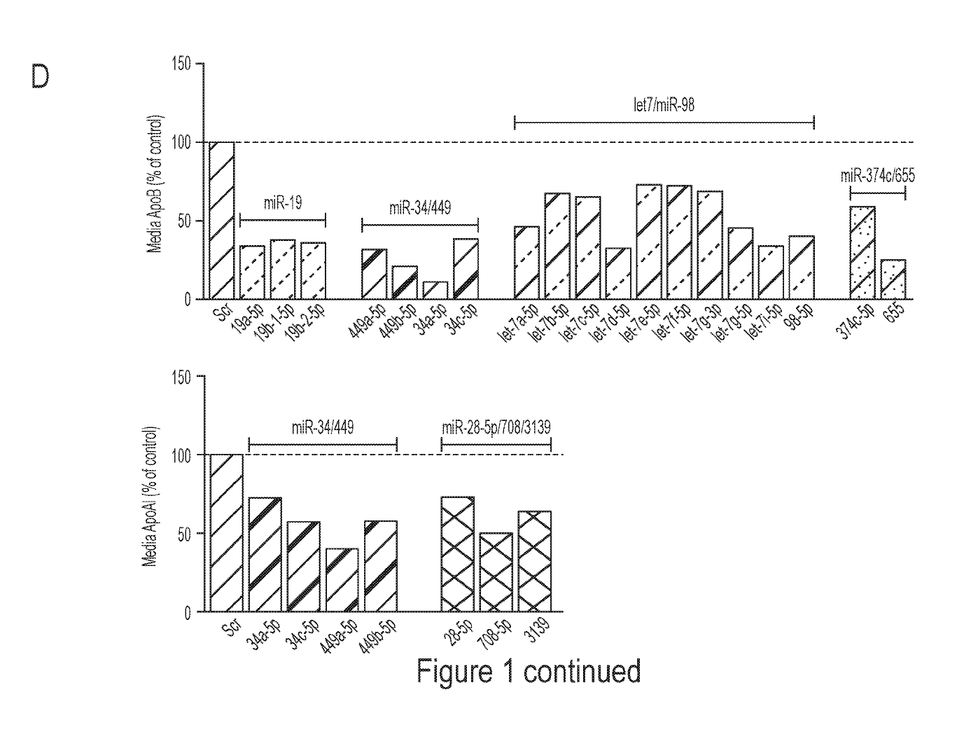

apoB and apoAI concentrations in medium were measured by ELISA (Hussain et al., 1995). Secreted apolipoproteins were quantified by ELISA as shown in FIG. 1A. For intracellular apoB measurement, cells were homogenized in 100 mM Tris buffer (pH7.4) containing 150 mM NaCl, 1 mM EGTA, 1 mM EDTA, 1% Triton X-100 and 0.5% SDS. apoB was measured via ELISA (Walsh et al., 2015).

The first screening performed in duplicate plates showed high reproducibility (Spearman r=0.96 and 0.92; FIG. 1B) and resulted in the identification of 60 and 57 miRs that decreased and increased, respectively, apoB secretion by over 50%; and 34 and 38 miRs that decreased and increased apoAI secretion by over 35% (FIG. 1C). Within these miRs, miRs in the same families exhibited similar effects on apoB and apoAI secretion indicating good internal reproducibility (FIG. 1D). In the second screening of 102 candidate miRs, 75 miRs gave results similar to the first screening. miR-1200 decreased apoB secretion by 61±2%, and increased apoAI secretion by 54±17%.

### Example 2

### MiR-1200 Decreases apoB Secretion by Enhancing Posttranscriptional mRNA Degradation

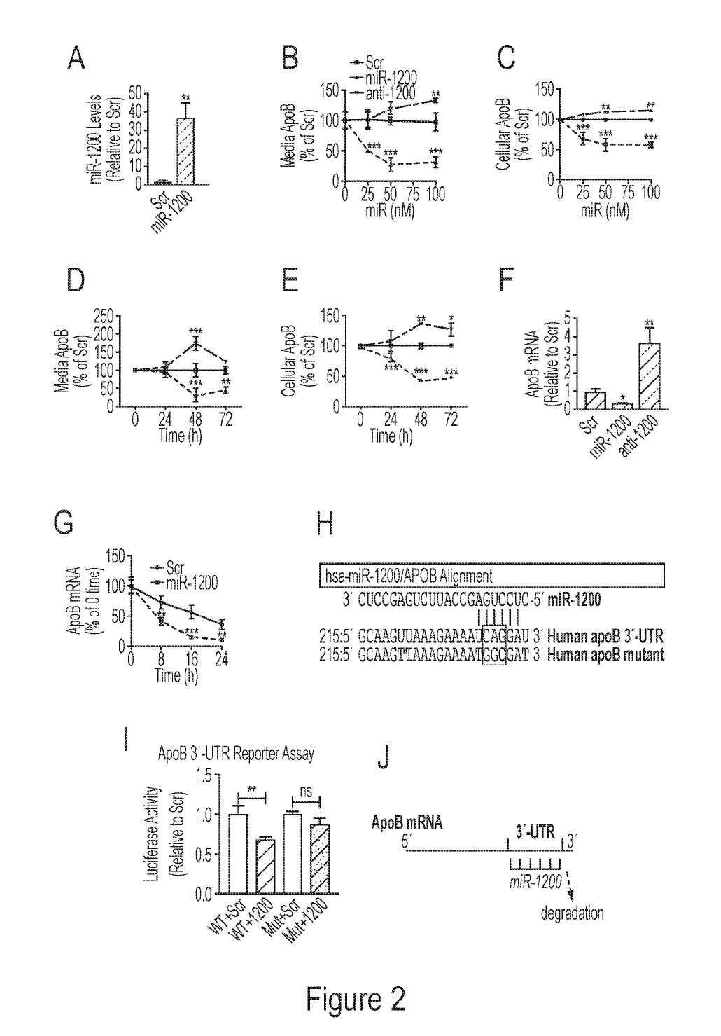

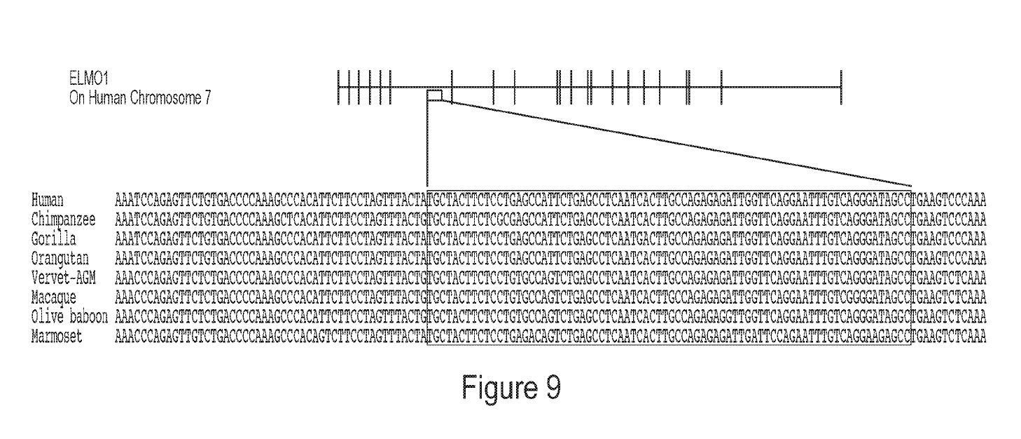

Hsa-miR-1200 is located in the 6^{th} intron of Engulfment and cell motility protein 1 (ELMO1) on human chromosome 7, and the precursor miR-1200 is conserved (FIG. 9). To study its role, Huh-7 cells were transfected with miR-1200 to increase cellular concentrations (FIG. 2A). MiR-1200 decreased media and cellular apoB in a dose-dependent manner (FIGS. 2B, 2C). Hairpin inhibitor of miR-1200, anti-1200, dose-dependently increased apoB suggesting that endogenous miR-1200 regulates apoB production (FIGS. 2B, 2C). The effects of miR-1200 and anti-1200 on cellular and media were maximum at 48 hours post transfection (FIGS. 2D, 2E). These studies showed that miR-1200 reduces, whereas anti-1200 increases, cellular and media apoB.

Lower cellular apoB protein levels could be due to reductions in mRNA or protein synthesis. Quantifications revealed that apoB mRNA levels were reduced in miR-1200 and increased in anti-1200 over-expressing cells, suggesting that miR-1200 modulates mRNA levels (FIG. 2F). To investigate how miR-1200 reduces apoB mRNA, mRNA degradation was determined after treating cells with actinomycin D to inhibit transcription. apoB mRNA disappeared faster in miR-1200 expressing cells (FIG. 2G), indicating that miR-1200 enhances posttranscriptional degradation. mRNA half-life was measured as follows: Huh-7 cells (1.2*10⁵/well) in 12-well plates were reverse transfected with miR-1200 or Scr (50 nM). After 24 hours, cells were treated with 1 μg/mL actinomycin D in growth medium. Total RNA were collected at different time points to quantify mRNA levels by qRT-PCR Primers used for qRT-PCR are shown in Table 2.

RNA isolation and qRT-PCR: Total RNA from tissues and cells was extracted using TRIzol (Invitrogen). RNA was reverse transcribed into cDNA with the Omniscript RT kit (QIAGEN). Expression levels of gene are quantified by qRT-PCR using SYBER Green qPCR Core Kit (Eurogentec), and data was analyzed with ΔΔCT method and normalized to 18S. Primers specific for miR-1200, miR-30c, snoRNA 202 were purchased from Life Technologies.

TABLE 2
Primers used for quantitative PCR

| Gene | Forward primer | Reverse primer |
|---|---|---|
| Human apoAI | 5′-GCAGAGACTATGTGTCCCAGTTTG-3′ | 5′-CCAGTTGTCAAGGAGCTTTAG-3′ |
| Human apoB | 5′-TGACCTTGTCCAGTGAAGTC-3′ | 5′-GTTCTGAATGTCCAGGGTGA-3′ |
| Human ABCA1 | 5′-TGGTCTCCAAGCAGAGTGTG-3′ | 5′-GAGCAGCAGCTCCCAATAC-3′ |
| Human BCL11B | 5′-CACCCCCGACGAAGATGACCAC-3′ | 5′-CGGCCCGGGCTCCAGGTAGATG-3′ |
| Human NCOR1 | 5′-CTGACAGGCCTCAAGAAAGG-3′ | 5′-AACCTGTTCCAGACGTGGTC-3′ |
| Mouse apoAI | 5′-GGCCGTGGCTCTGGTCTT-3′ | 5′-GGTTCATCTTGCTGCCATACC-3′ |
| Mouse apoB | 5′-CTCGACCATCGGCACTGT-3′ | 5′-AGTTTCTTCTCTGGAGGGGACT-3′ |
| Mouse MTP | 5′-CACACAACTGGCCTCTCATTAAAT-3′ | 5′-TGCCCCCATCAAGAAACACT-3′ |
| Mouse ABCA1 | 5′-TTGGCGCTCAACTTTTACGAA-3′ | 5′-GAGCGAATGTCCTTCCCCA-3′ |
| Mouse BCL11B | 5′-GAGCCCTTTCCAGCTCTCTT-3′ | 5′-CCAGGTCTTTCTCCACCTTG-3′ |
| Mouse ABCG1 | 5′-ACAACTTCACAGAGGCCCAG-3′ | 5′-TTTCCCAGAGATCCCTTTCA-3′ |
| Mouse SR-BI | 5′-ACGGCCAGAAGCCAGTAGTC-3′ | 5′-GACCTTTTGTCTGAACTCCCTGTAG-3′ |
| Mouse NCOR1 | 5′-AGAACTTCTGATGTTTCTTCCAG-3′ | 5′-CTGGAGACTTGGCTGGTATA-3′ |
| Mouse CPT1A | 5′-AAGCACCAGCACCTGTACCG-3′ | 5′-CCTTTACAGTGTCCATCCTCTG-3′ |
| Mouse ACOX1 | 5′-AAGAGTTCATTCTCAACAGCCC-3′ | 5′-CTTGGACAGACTCTGAGCTGC-3′ |
| Mouse MCAD | 5′-TTACCGAAGAGTTGGCGTATG-3′ | 5′-ATCTTCTGGCCGTTGATAACA-3′ |
| Mouse PGC-1a | 5′-ATACCGCAAAGAGCACGAGAAG-3′ | 5′-CTCAAGAGCAGCGAAAGCGTCACAG-3′ |
| 18s rRNA | 5′-AGTCCCTTGCCCTTTGTACACA-3′ | 5′-GATCCGAGGGCCTCACTAAAC-3′ |

TABLE 3
Primers used for site-directed mutagenesis
apoB _{C231G}_A232G_G233C
Forward: 5′-TAGCAAAATAACTCAGATCGCCATTTTCTTTAACTTGCAAAAAATGCCATCCTTCTG-3′
Reverse: 5′-CAGAAGGATGGCATTTTTTGCAAGTTAAAGAAAATGGCGATCTGAGTTATTTTGCTA-3′

TABLE 4
siRNAs (Dharmacon)

| Catalog | Gene | Gene | |
|---|---|---|---|
| Number | Symbol | Accession | Sequence |
| D-006686- | NRIP1 | NM_003489 | SEQ ID NO: 3: |
| 01 | | | GAACAAAGGUCAUGAGUGA |
| D-005082- | BCL11B | NM_022898 | SEQ ID NO: 4: |
| 01 | | | GAGCAAGUCGUGCGAGUUC |
| D-020818- | ZBTB7A | NM_015898 | SEQ ID NO: 5: |
| 01 | | | UCACCGCGCUCAUGGACUU |

The mechanism by which miR-1200 regulates apoB mRNA degradation was further elucidated by in silico analysis using miRanda (http://www.microrna.org/microrna/home.do), showing that apoB mRNA contains a miR-1200 interacting site in its 3′-UTR (FIG. 2H). This indicates that miR-1200 interacts with the 3′-UTR of apoB mRNA to increase degradation. DNA encoding the 3′-UTR of human apoB mRNA was inserted after the luciferase cDNA in psiCHECK2 plasmid by standard cloning methods to obtain pLuc-apoB-3′-UTR expression plasmid. This plasmid or control psiCHECK2 plasmid (1.5 μg) was transfected using TurboFect transfection reagent (Dharmacon) in Huh-7 cells (1.2*106) plated in 10 cm Petri dishes one day before transfection. After 24 hours of transfection, cells were detached and plated in 6-well plates containing miRs+RNAiMAX for reverse transfection (final concentration: 50 nM). Luciferase activity was measured after 48 hours with Dual-Luciferase Reporter Assay System (Promega). apoAI promoter luciferase reporter construct was purchased from GeneCopoeia. Luciferase activity of this plasmid was significantly reduced by miR-1200 and this inhibition was avoided after mutagenesis of the complementary site that interacts with the seed sequence (FIG. 2I). These results indicate that miR-1200 interacts with the 3′-UTR of apoB to increase mRNA degradation (FIG. 2J).

### Example 3

### MiR-1200 Increases apoAI Secretion by Reducing BCL11B, a Repressor of apoAI Transcription

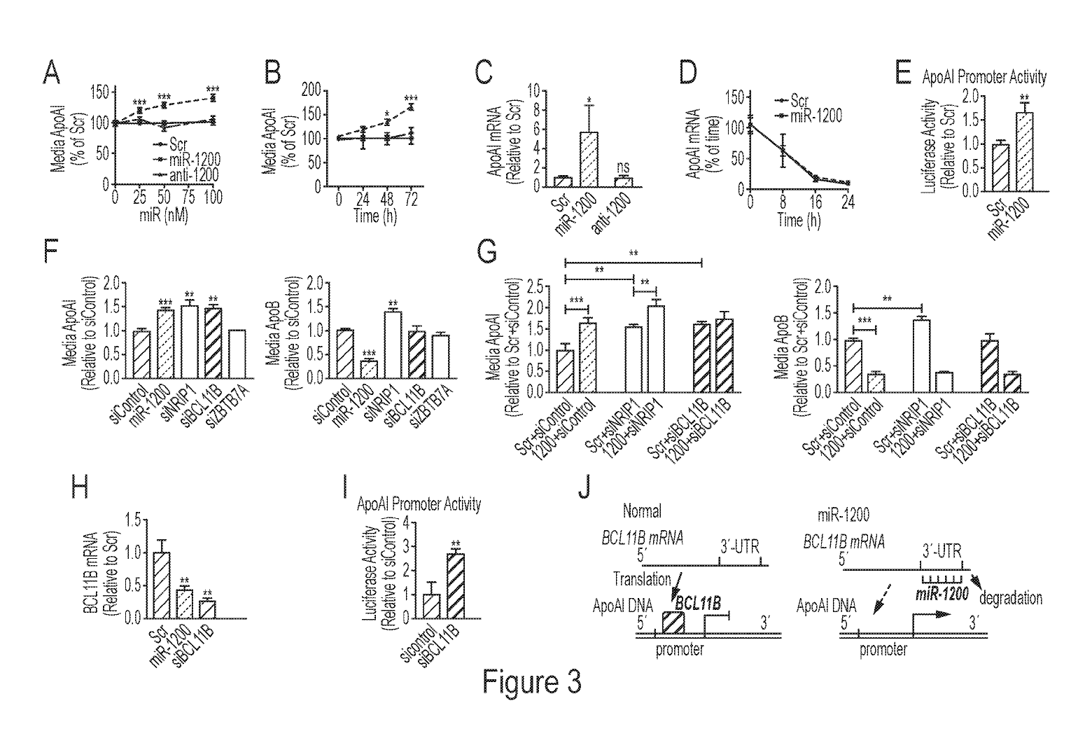

The following example demonstrates that miR-1200 increases apoAI secretion by reducing BCL11B, a repressor of apoAI transcription. MiR-1200 dose-dependently enhanced apoAI secretion by ˜41% in Huh-7 cells compared to Scr (FIG. 3A). Time course studies showed that media apoAI continued to increase until 72 hours after miR-1200 transfection (FIG. 3B). MiR-1200 increased apoAI mRNA by ˜6-fold (FIG. 3C). Therefore, overexpression of miR-1200 increases media apoAI by elevating mRNA levels. In these studies, anti-1200 had no effect on apoAI expression (FIGS. 3A-C) indicating a complex mode of apoAI regulation different from that of apoB. MiR-1200 had no effect on apoAI mRNA degradation (FIG. 3D). However, it increased the activity of a 1.2 kb apoAI promoter by ˜67% (FIG. 3E) demonstrating that miR-1200 increases apoAI mRNA by enhancing transcription.

Although miRs normally reduce gene expression (He and Hannon, 2004), they have been shown to activate transcription by interacting with promoter sequences involving complementary base pairing via RNA activation (Huang et al., 2012; Place et al., 2008). There were no miR-1200 complementary sequences in the 1.2-kb apoAI promoter. To determine whether miR-1200 may instead increase apoAI transcription by suppressing a transcriptional repressor(s), three transcriptional repressors were selected from a list of predicted miR-1200 target genes generated by TargetScan (http://www.targetscan.org/) as they had the potential to bind the apoAI promoter, and the target sites were conserved in human and mouse. Huh-7 cells were then transfected with siRNAs against NRIP1 (Nuclear Receptor Interacting Protein 1), BCL11B (B-Cell Lymphoma 11B), or ZBTB7A (Zinc Finger and BTB Domain Containing 7A) (FIG. 3F). As expected, miR-1200 reduced apoB; however siNRIP1 (SEQ ID NO:3, Table 4) increased apoB secretion while siBCL11B and siZBTB7A had no effect on apoB indicating that these repressors do not regulate apoB secretion like miR-1200. However, similar to miR-1200, both siNRIP1 and siBCL11B increased media apoAI by about ˜46-53%, but siZBTB7A had no effect. Therefore, NRIP1 and BCL11B may work as apoAI repressors.

To test whether BCL11B is an intermediary in the regulation of apoAI by miR-1200, miR-1200 was co-transfected with siRNAs in Huh-7 cells (FIG. 3G). MiR-1200 and siNRIP1 alone increased apoAI secretion by 64 and 50%, respectively, while a combination of both miR-1200 and siNRIP1 increased apoAI secretion by 104% compared to Scr+siControl. This suggests that NRIP1 and miR-1200 additively increase apoAI secretion by possibly involving two independent mechanisms (FIG. 3G). On the other hand, miR-1200, siBCL11B and siBCL11B+miR-1200 increased apoAI to similar levels (FIG. 3G). In contrast, miR-1200 reduced apoB secretion in cells treated with both siNRIP1 and siBCL1B. These data show that miR-1200 is unable to increase apoAI secretion in siBCL11B treated cells but is able to reduce apoB secretion. Thus, miR-1200 increases apoAI expression indirectly by reducing expression of its repressor, BCL11B.

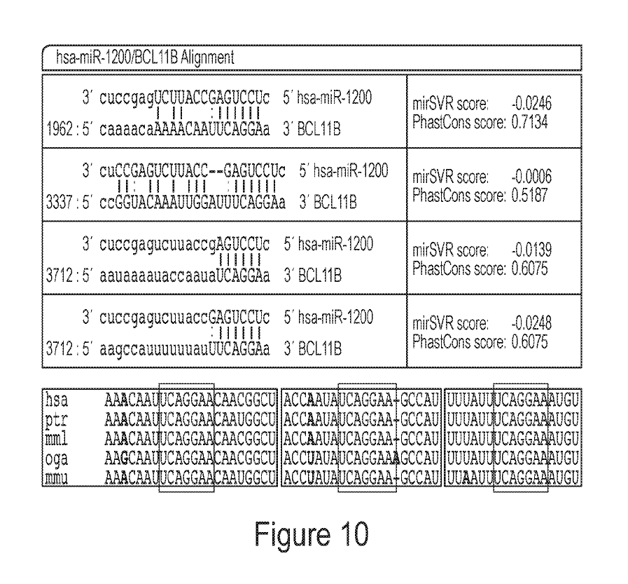

Bioinformatics analyses showed that the 3′-UTR of the human BCL11B mRNA contained 4 miR-1200 binding sites and 3 of these sites were evolutionarily conserved (FIG. 10). To test whether miR-1200 regulates BCL11B, mRNA levels were quantified in miR-1200 transfected cells. BCL11B mRNA levels were decreased by ˜56% in miR-1200 and siBCL11B expressing cells (FIG. 3H), indicating that miR-1200 regulates BCL11B expression. Further, siBCL11B increased apoAI promoter activity by ˜2.6-fold (FIG. 3I), suggesting that BCL11B represses apoAI transcription. These studies indicate that miR-1200 increases apoAI expression by reducing BCL11B (FIG. 3J).

### Example 4

### MiR-1200 Reduces apoB and Increases apoAI in Other Human and Mouse Hepatoma Cell Lines

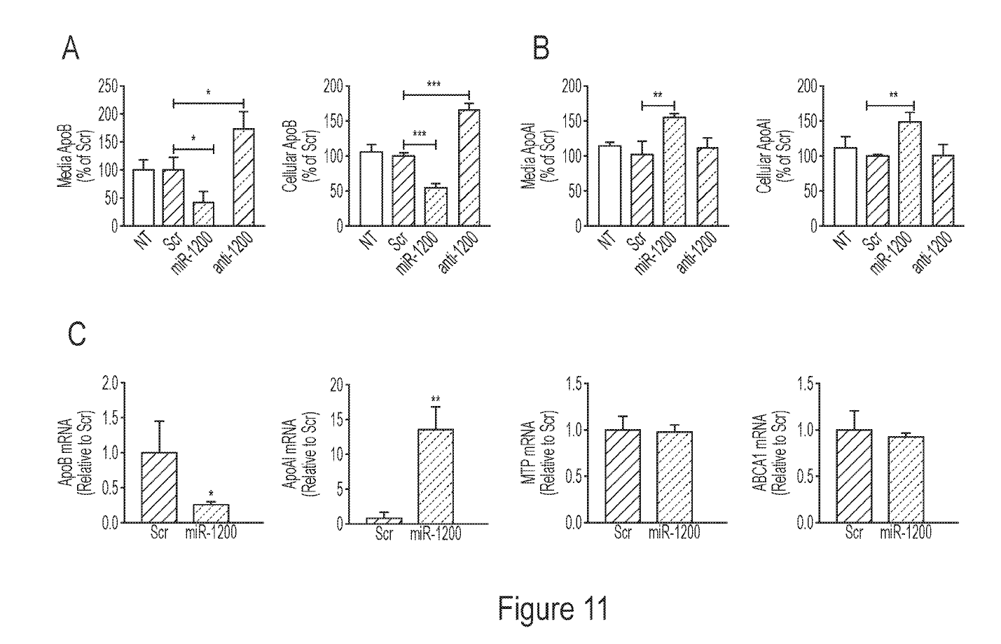

To ascertain that the regulation of apoB and apoAI by miR-1200 is not specific to Huh-7 cells, its effects in other human hepatoma HepG2 cells were studied. MiR-1200 and anti-1200 decreased and increased media and cellular apoB, respectively (FIG. 11A). Further, miR-1200 increased media and cellular apoAI levels by ˜48-54% while anti-1200 had no effect (FIG. 11B). These studies showed that miR-1200 regulates apoB and apoAI levels in HepG2 cells.

Since mouse models are commonly used to evaluate the role of miRs in lipid metabolism and atherosclerosis, the effects of miR-1200 on apoB and apoAI in mouse hepatoma AML12 cells were examined. Expression of miR-1200 decreased apoB and increased apoAI but had no effect on MTTP and ABCA1 mRNA levels (FIG. 11C). Thus, miR-1200 also modulates apoB and apoAI expression in mouse hepatoma cells.

### Example 5

### MiR-1200 Reduces LDL and Increases HDL Cholesterol in Western Diet Fed C57BL/6J Mice

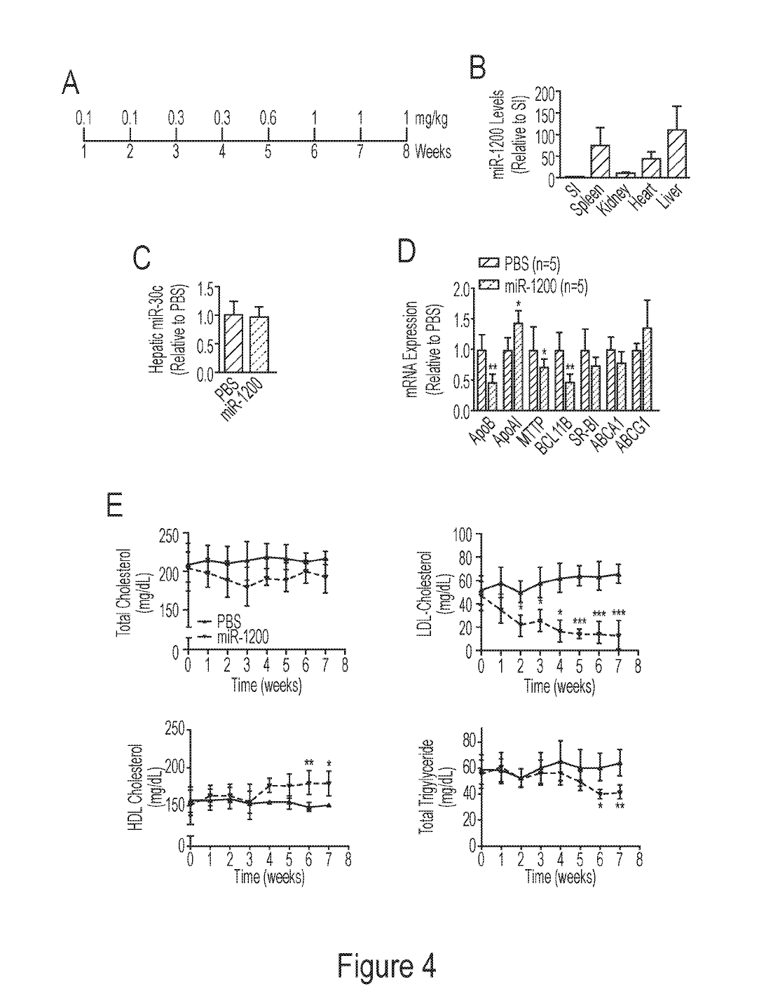

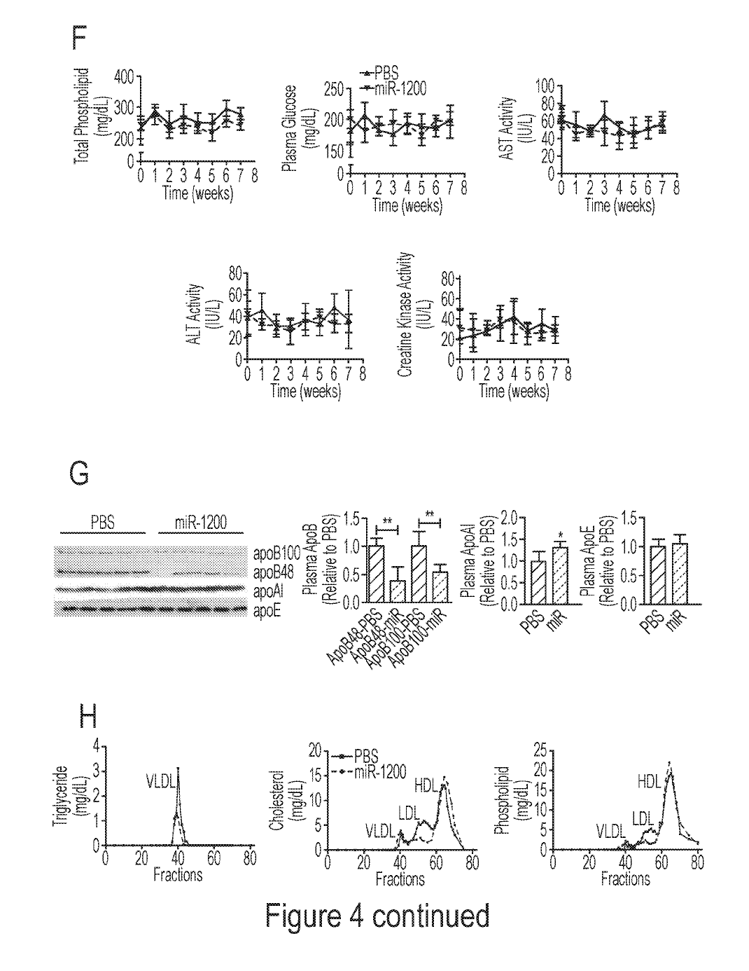

To investigate the physiological consequences of miR-1200 overexpression, a dose-escalation study in wild type C57BL/6J mice fed a Western diet for 6 weeks was performed (FIG. 4). Mice were first injected with a low dose of miR-1200 (0.1 mg/kg/week) or PBS control. Dosage was increased gradually in the following weeks to 0.3 mg/kg, 0.6 mg/kg and 1 mg/kg per week (FIG. 4A). At the end of the study, tissue distribution studies in miR-1200 injected mice showed that liver, spleen and heart contained significant amounts of miR-1200 (FIG. 4B). The effects of miR-1200 overexpression in the liver were further investigated. The hepatic accretions of miR-1200 had no effect on the endogenous miR-30c levels (FIG. 4C). MiR-1200 significantly reduced hepatic apoB and BCL11B, increased apoAI, and had no effect on MTTP, SR-BI, ABCA1 and ABCG1 mRNA levels (FIG. 4D). These studies indicate that miR-1200 accumulated in the liver and reduced the expression of its target genes, but had no effect on non-target genes.

Analysis of Plasma Constituents

Blood was collected in EDTA containing tubes from overnight fasted mice. Plasma was separated by centrifugation. Total plasma cholesterol, triglyceride, and phospholipid were measured using commercial kits (Thermo Fisher Scientific, Wako Diagnostic). To precipitate apoB-containing lipoproteins, 25 μL of 0.44 mM phosphotungstic acid and 20 mM MgCl₂ were added to 10 μL of plasma, incubated for 5 min at room temperature, and centrifuged at 12,000*g. Supernatants were used to measure cholesterol in HDL. Cholesterol levels in non-HDL fractions were determined by subtracting HDL-cholesterol from total cholesterol. Lipids were extracted from liver homogenates using methanol/chloroform and quantified using kits. Plasma ALT, AST, glucose and CK were measured using commercial available kits (Pointe Scientific, Wako Diagnostic, and Thermo scientific) according to the manufacturer's instructions.

Analyses of plasma constituents revealed no significant changes in total plasma cholesterol (FIG. 4E). However, miR-1200 reduced non-HDL (LDL) cholesterol at the lowest 0.1 mg/kg/week dose and the effect persisted at higher doses (FIG. 4E). Low dose of miR-1200 had no effect but higher doses of 0.6 and 1 mg/kg/week increased HDL-cholesterol compared with the PBS group (FIG. 4E). At low doses, total plasma triglyceride did not change. However, at a 1 mg/kg/week dose, plasma triglycerides were significantly reduced. At all the doses, there were no significant differences in plasma phospholipids, glucose, aspartate aminotransferase (AST), alanine aminotransferase (ALT), and creatine kinase (CK) levels in these two groups (FIG. 4F). Western blot of plasma proteins showed that miR-1200 reduced apoB48 and apoB100 by 60 and 48%, respectively; increased apoAI by 32%; and had no effect on apoE (FIG. 4G). Gel filtration showed that triglyceride in VLDL fractions was decreased, cholesterol and phospholipid were reduced in the LDL fraction, and cholesterol and phospholipid were increased in HDL of the miR-1200 group compared to controls (FIG. 4H). These studies showed that miR-1200 reduces non-HDL cholesterol and increases HDL cholesterol without causing liver or muscle toxicity. Thus, miR-1200 differentially modulates plasma lipoproteins with no obvious adverse effects.

### Example 6

### MiR-1200 Does Not Cause Hepatosteatosis and Increases Fatty Acid Oxidation (FAO)

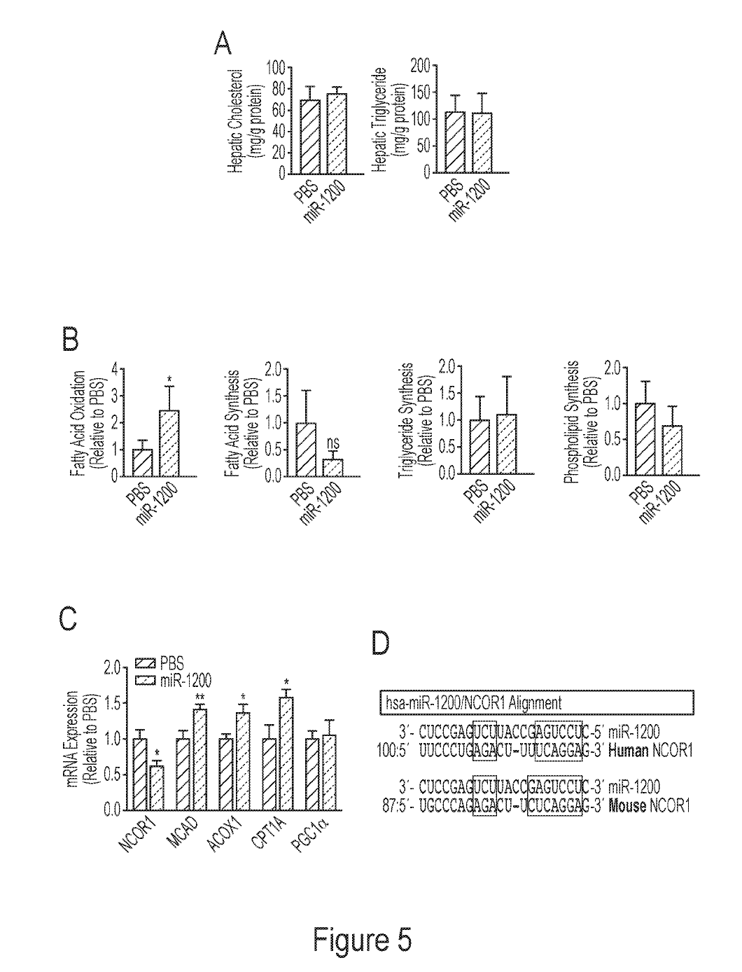

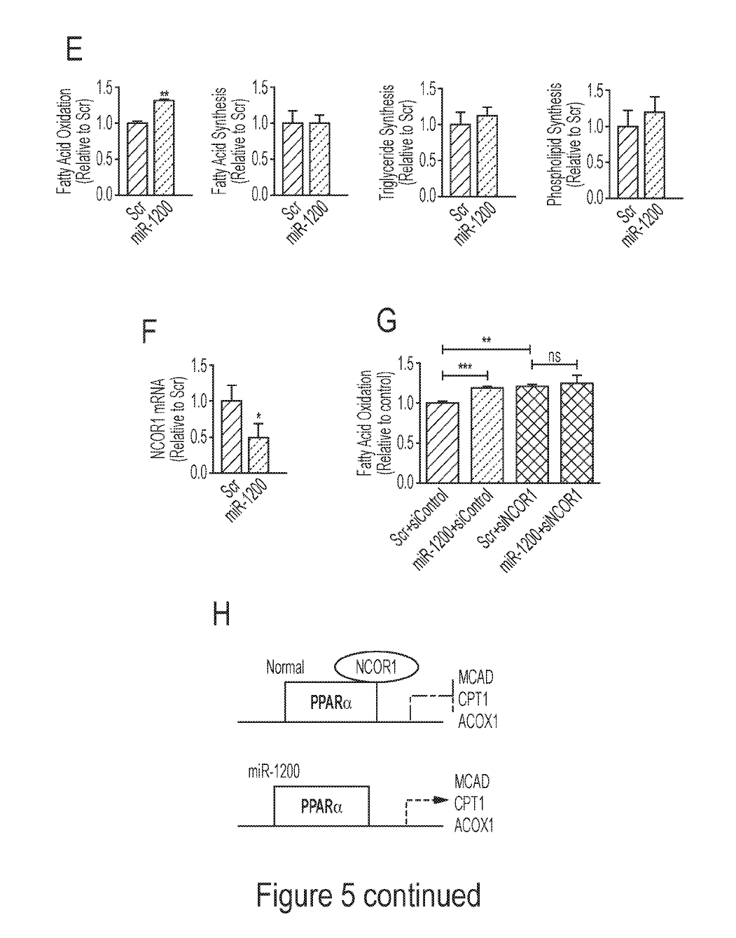

The effects of miR-1200 on hepatic lipid metabolism were tested. Assimilation of miR-1200 in the liver had no effect on hepatic cholesterol and triglyceride levels, indicating that miR-1200 does not cause hepatic steatosis (FIG. 5A). Increased hepatic lipid content is usually observed when apoB-lipoprotein secretion is reduced via MTP inhibition or the use of apoB anti-sense. Previous studies show that miR-30c inhibits hepatic lipoprotein production but does not cause steatosis by reducing lipid synthesis (Soh et al., 2013).

The effect of miR-1200 on lipid synthesis and FAO was assessed. In the livers of miR-1200 injected group, FAO was increased by >2-fold but had no effect on the synthesis of different lipids (FIG. 5B). Consistent with increases in FAO, these livers had higher expression levels of MCAD, ACOX1, and CPT1 and lower NCOR1 levels, a known repressor of FAO (Fan and Evans, 2015; Mottis et al., 2013) (FIG. 5C). Prediction algorithms informed that NCOR1 is a target of miR-1200 (FIG. 5D). To test whether miR-1200 regulates FAO by modulating NCOR1 levels, Huh-7 cells were transfected with Scr or miR-1200. Transfection of miR-1200 increased FAO without affecting lipid syntheses (FIG. 5E) and reduced NCOR1 mRNA levels (FIG. 5F). To determine whether miR-1200 regulates FAO via NCOR1, Huh-7 cells were co-transfected with different combinations of miRs and siRNAs (FIG. 5G). MiR-1200 and siNCOR1 significantly increased FAO. siNCOR1+miR-1200 increased FAO to similar extents indicating that miR-1200 and NCOR1 are in the same pathway and that miR-1200 might reduce NCOR1 to increase FAO. Under normal conditions, NCOR1 interacts with PPARα/RXR to reduce the expression of genes involved in FAO. Overexpression of miR-1200 may reduce NCOR1 levels de-repressing the expression of genes involved in FAO (FIG. 5H).

Fatty acid oxidation and synthesis of fatty acids, triglycerides, and phospholipids: For hepatic FAO, ˜100 mg fresh liver slices were incubated with 0.2 μCi of ¹⁴C-oleate for 2 h. Released ¹⁴C—CO₂ was trapped in phenylethylamine soaked Whatman filter paper and counted (Khatun et al., 2012; Soh et al., 2013). To study FAO in cells, Huh-7 cells were plated in 12-well plates and incubated with DMEM containing 0.4 μCi/ml of ¹⁴C-oleate and covered with phenylethylamine soaked Whatman filter paper for 3 hours at 37° C. At the end of incubation, 200 μl of 1M perchloric acid was added to media and incubated for 1 h at room temperature to precipitate acid-insoluble metabolites, and centrifuged (10 min 12,000*g). The radioactivity in the supernatant and the filter paper was counted.

For fatty acid synthesis (de novo lipogenesis), about 50 mg fresh liver slices were incubated with 1 μCi ¹⁴C-acetate. After one hour, the liver slices were washed with PBS and subjected to fatty acids extraction using Petroleum Ether. The radioactivity in fatty acids was measured by scintillation counter. For triglyceride and phospholipid synthesis, 50 mg fresh liver slices were labeled with 1 μCi of ³H-glycerol for 1 hour. Total lipids were extracted by chloroform and methanol and separated on silica-60 Thin Layer Chromatography. The bands containing triglyceride or phospholipid were scraped off from the plates and counted in a scintillation counter.

### Example 7

### MiR-1200 Reduces Hepatic Production of apoB-Containing Lipoproteins and Augments Reverse Cholesterol Transport

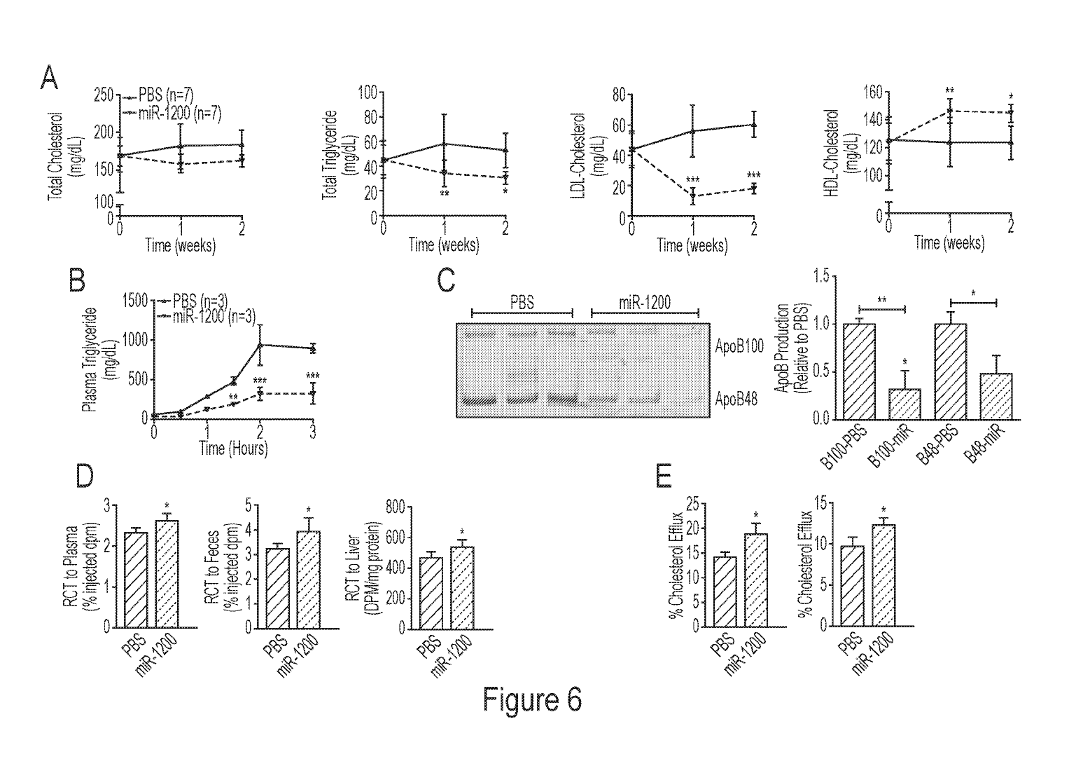

MiR-1200 significantly reduced plasma LDL cholesterol levels (FIG. 4E) and cellular and media apoB (FIGS. 2A-D, FIGS. 11A-11C). Additionally, miR-1200 increased plasma HDL (FIG. 4E). The following example assesses whether (1) miR-1200 reduces hepatic VLDL production to lower plasma LDL and (2) miR-1200 enhances RCT from lipid-loaded macrophages to plasma, liver and feces. Western diet-fed male C57BL/6J mice were injected with 1 mg/kg/week miR-1200 or PBS for two weeks. As before, miR-1200 had no effect on total cholesterol, but decreased total triglyceride levels (FIG. 6A). Quantifications of cholesterol in different lipoproteins showed that miR-1200 decreased LDL-cholesterol and increased HDL cholesterol (FIG. 6A). After the second weekly injection, mice were divided into two groups and used for VLDL production and RCT. For VLDL production, overnight fasted mice were injected intraperitoneally with poloxamer 407 (1 mg/g body weight) and 150 μCi of [35S]Promix (Soh et al., 2013) to inhibit lipoprotein lipase. Blood was removed at indicated time points. apoB was immunoprecipitated, separated on SDS-PAGE, and visualized by autoradiography. For immunoprecipitation, plasma (100 μl) was incubated for 16 h with 5 μl of anti-apoB polyclonal antibody (Texas Academy Biosciences, Product ID 20A-G1) in NET buffer (50 mM Tris, pH 7.4, 150 mM NaCl, 5 mM EDTA, 0.5% Triton X-100 and 0.1% SDS) and Protein A/G PLUS-Agarose beads (Sigma, sc-2003).

MiR-1200 injected mice accumulated reduced amounts of triglyceride in plasma over time (FIG. 6B). Triglyceride production rates were 3-fold lower in the miR-1200 group (138 mg·dL⁻¹·hour⁻¹) compared with the PBS group (432 mg·dL⁻¹·hour⁻¹). Additionally, the amounts of newly synthesized apoB were significantly reduced in the plasma of miR-1200 group (FIG. 6C). These studies indicate that miR-1200 significantly diminishes hepatic production of triglyceride-rich apoB-containing lipoproteins.

For in vivo RCT (McGillicuddy et al., 2009), mice were injected with ³H-cholesterol labeled macrophages. After 48 hours, miR-1200 treated mice had 13% more ³H-cholesterol in plasma, 22% more in feces, and 16% more in the liver compared with PBS controls (FIG. 6D). These studies indicated that miR-1200 enhances RCT from macrophages.

For RCT (Rohatgi et al., 2014; Khera et al., 2011; McGillicuddy et al., 2009), J774A.1 cells (10⁵/well) were plated in 6-well plates one day before loading. For loading, cells were incubated with Ac-LDL (50 μg/ml)+³H-cholesterol (5 μCi/ml) in DMEM containing 10% FBS for 48 hours. After washing with PBS three times, cells were incubated with 0.5% BSA containing DMEM for one hour. Cells were harvested, washed, and suspended in 0.5% BSA containing DMEM. A small aliquot of cells was counted in scintillation counter to measure the total injected dpm. 300 μl of cells were injected into each mouse. Samples were collected after 48 hours.

Increases in RCT are due to augmentations in cholesterol efflux potential of HDL. Total plasma and HDL isolated from miR-1200 treated mice effluxed ˜26% more radiolabeled cholesterol than controls (FIG. 6E). These data showed that HDL levels increased by miR-1200 are efficient in cholesterol efflux.

Cholesterol Efflux Assay

For cholesterol efflux (Khera et al., 2011), J774A.1 cells (1.2×10⁴) were plated in each well of a 96-well plate one day before loading. For loading, cells were incubated with DMEM containing 50 μg/mL Ac-LDL, 0.2 μCi/mL ³H-cholesterol, 10% FBS and 0.5% BSA for 48 hours. Then cells were washed three times with PBS and equilibrated in serum free DMEM containing 2 μM of LXR agonist TO901317 and 0.5% BSA for 24 hours. HDL or whole plasma (5%, v/v) was used as cholesterol acceptor. DMEM containing 0.5% BSA was used as control. The efflux was performed in the presence of TO901317 and 0.5% BSA for 6 hours. After efflux, radiolabeled cholesterol in media and cells were counted separately. Percent Cholesterol efflux=media counts/(media counts+cell counts)*100% −% blank efflux.

### Example 8

### MiR-1200 Reduces Atherosclerosis in Apoe^{-/-} Mice

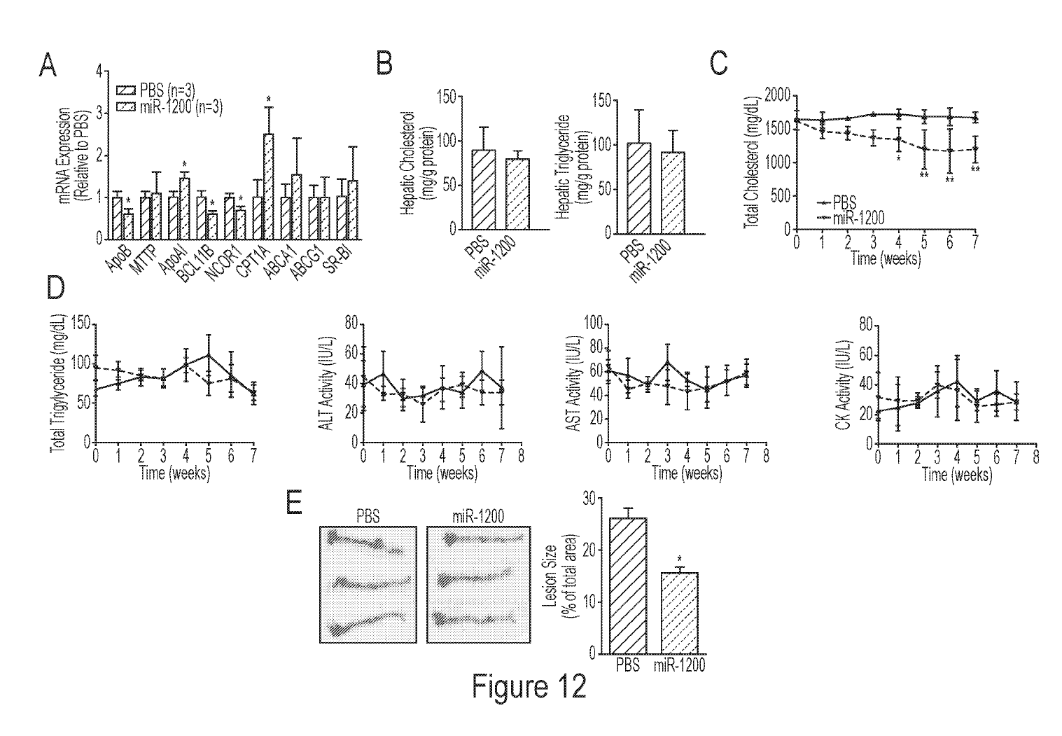

This example demonstrates that miR-1200 can reduce atherosclerosis. Western diet fed Apoe^{-/-} mice were injected with 1 mg/kg/week miR-1200 for 7 weeks (FIGS. 12A-12E). Injection of miR-1200 significantly reduced hepatic apoB, BCL11B, and NCOR1; increased ApoAI, and CPT1; and had no effect on MTTP, ABCA1, and ABCG1 mRNA levels compared with controls (FIG. 12A). Lipid analyses revealed no significant differences in hepatic cholesterol and triglyceride in both the groups (FIG. 12B). Plasma total cholesterol significantly reduced starting from week 4 (FIG. 12C). There were no significant changes in plasma triglyceride, ALT, AST and CK levels (FIG. 12D). Oil Red O staining of aortas showed significantly reduced lesion size in the miR-1200 group (FIG. 12E). These studies indicated that miR-1200 reduces atherosclerotic plaques.

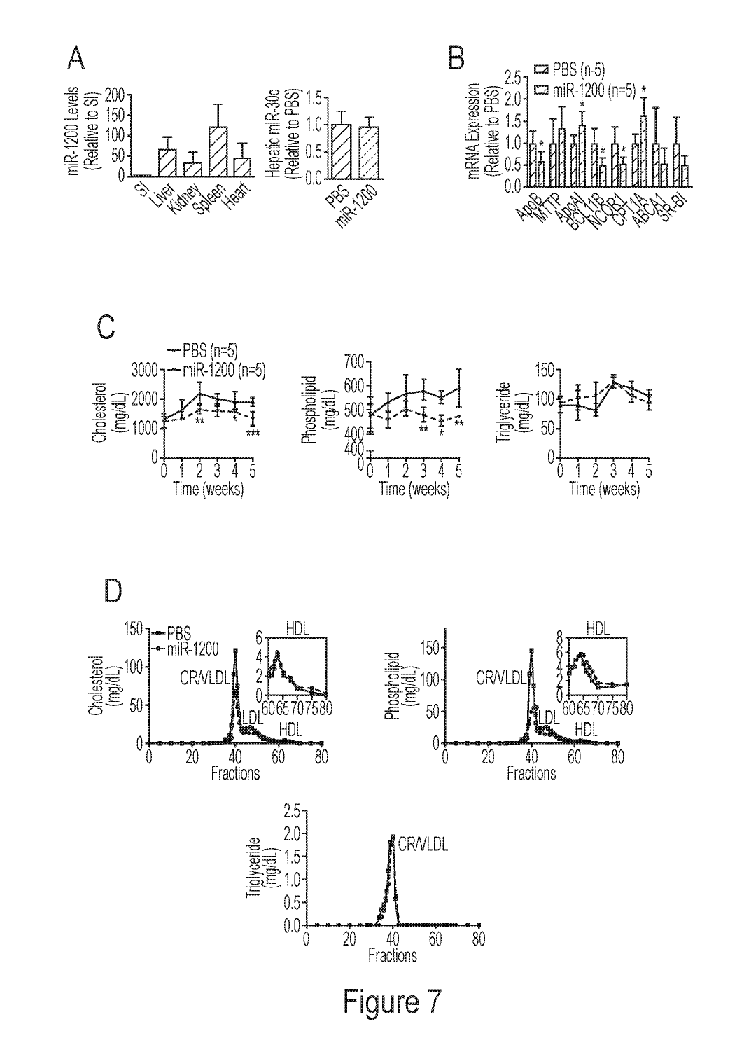

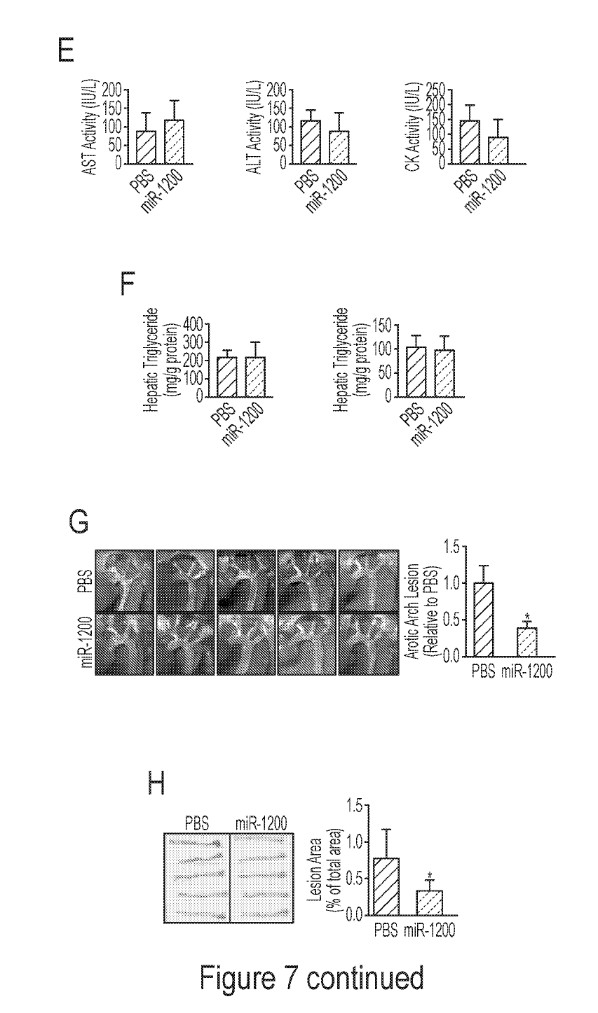

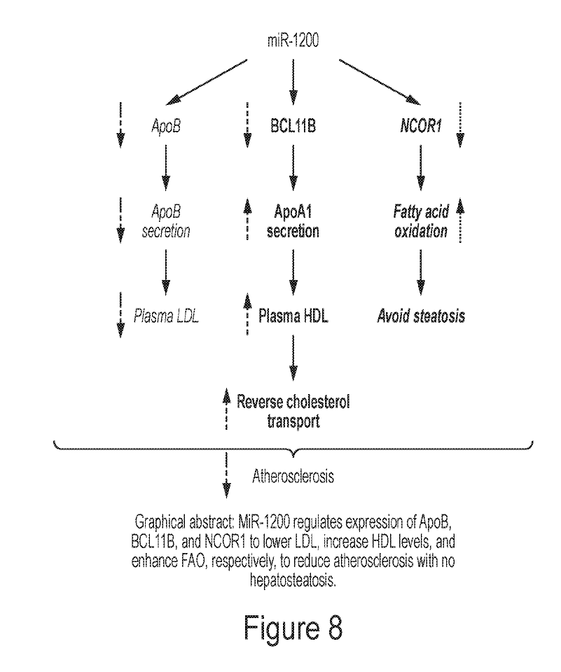

In a second experiment, mice fed a Western diet were injected with 2 mg/kg/week of miR-1200 or PBS for 5 weeks. Again, miR-1200 accumulated in the liver, kidney, spleen and heart of these mice and hepatic accretions had no effect on miR-30c expression (FIG. 7A). The mRNA levels of apoB, BCL11B and NCOR1 were significantly reduced, apoAI and CPT1 were increased, and MTTP, SR-B1 and ABCA1 were not changed (FIG. 7B). Total cholesterol and phospholipids in plasma were significantly decreased by miR-1200 treatment, while plasma triglyceride levels were unaffected (FIG. 7C). FPLC analyses showed that reductions in cholesterol and phospholipids were mainly in apoB-containing lipoproteins (FIG. 7D). Again, liver and muscle injury markers (ALT, AST and CK) were not elevated in plasma (FIG. 7E). Further, miR-1200 did not cause lipid accumulation in the liver, as hepatic cholesterol and triglyceride were the same as in the control group (FIG. 7F). In miR-1200 injected Apoe^{-/-} mice, aortic arch lesions were significantly reduced (FIG. 7G). Further, lipid accumulation in the aortas determined by Oil Red O staining was significantly lower in the miR-1200 group (FIG. 7H). Therefore, these studies show that miR-1200 decreases total plasma cholesterol levels and reduces atherosclerosis in Apoe^{-/-} mice.

### Example 9

### Cell Culture

Cells used in the foregoing Examples including, Human hepatoma Huh-7 and HepG2; mouse hepatoma AML12; and mouse macrophage J774A.1 cells from American Type Culture Collection were maintained in Dulbecco's Modified Eagle Medium (DMEM) containing 10% fetal bovine serum, 1% penicillin-streptomycin and 1% L-glutamine in a 37° C., 5% CO2 cell culture incubator.

### Example 10

### Methods of Preventing and Treating Hyperlipidemia and Atherosclerosis in Human Patients

A therapeutically effective amount of a miR comprising SEQ ID NO:1 is administered to a human patient, wherein LDL is decreased and HDL is increased, without causing liver or muscle injury. The miR is administered at a dose of 0.1-2 mg/kg/week, with the specific dosage chosen based on the type and severity of the disease and patient response and characteristics. A dose as low as 0.1 mg/kg/week, i.e. a dose 10-fold lower than that used in mice, may be therapeutically effective, given the slower metabolic rate in humans. To achieve optimal response, the dose may be increased up to 1 mg/kg/week, the same dose as in mice. If needed, the dose may be further increased up to 2 mg/kg/week. Such dose optimization is within the skill of a person of ordinary skill in the art.

### Example 11

### Methods of Increasing apoAI levels by Reducing NRIP1

A therapeutically effective amount of an NRIP1 inhibitor is administered to cells in vitro or in vivo, thereby increasing the expression of apoAI. The inhibitor may be a nucleic acid inhibitor, such as an siRNA, e.g. with the sequence of SEQ ID NO:3, shown in Table 4. Alternatively, the NRIP1 inhibitor may be a small molecule or a protein, such as an antibody or a fusion protein. To further enhance apoAI expression, the NRIP1 inhibitor is optionally administered in combination with an inhibitor of BCL11B and/or an inhibitor of apoB expression or activity. When administered to an animal or a human patient, the specific dosage of each inhibitor is chosen and adjusted based on the type and severity of the disease, as well as the patient response and characteristics.

### REFERENCES

Chiou et al., Journal of Controlled Release. 2012; 159:240-25
Cornier, M. A. and Eckel, R. H. (2015). Non-traditional dosing of statins in statin-intolerant patients-is it worth a try? Curr. Atheroscler. Rep. 17, 475.
Fisher, E. A. and Ginsberg, H. N. (2002). Complexity in the secretory pathway: the assembly and secretion of apolipoprotein B-containing lipoproteins. J. Biol. Chem. 277, 17377-17380.
Khatun, I., Zeissig, S., Iqbal, J., Wang, M., Curiel, D., Shelness, G. S., Blumberg, R. S., and Hussain, M. M. (2012). Phospholipid transfer activity of MTP promotes assembly of phospholipid-rich apoB-containing lipoproteins and reduces plasma as well as hepatic lipids in mice. Hepatology 55, 1356-1368.
Khera, A. V., Cuchel, M., de, l.L.-M., Rodrigues, A., Burke, M. F., Jafri, K., French, B. C., Phillips, J. A., Mucksavage, M. L., Wilensky, R. L., Mohler, E. R., Rothblat, G. H., and Rader, D. J. (2011). Cholesterol efflux capacity, high-density lipoprotein function, and atherosclerosis. N. Engl. J. Med. 364, 127-135.
Liu et al., Molecular Pharmaceutics. 2011; 8:250-259
McGillicuddy, F. C., de la Llera, M. M., Hinkle, C. C., Joshi, M. R., Chiquoine, E. H., Billheimer, J. T., Rothblat, G. H., and Reilly, M. P. (2009). Inflammation impairs reverse cholesterol transport in vivo. Circulation 119, 1135-1145.
Medina et al., Curr Pharm Des. 2004; 10(24):2981-9
Rohatgi, A., Khera, A., Berry, J. D., Givens, E. G., Ayers, C. R., Wedin, K. E., Neeland, I. J., Yuhanna, I. S., Rader, D. R., De Lemos, J. A., and Shaul, P. W. (2014). HDL cholesterol efflux capacity and incident cardiovascular events. N. Engl. J. Med. 371, 2383-2393.
Soh, J., Iqbal, J., Queiroz, J., Fernandez-Hernando, C., and Hussain, M. M. (2013). MicroRNA-30c reduces hyperlipidemia and atherosclesrosis by decreasing lipid synthesis and lipoprotein secretion. Nat. Med. 19, 892-900.
Walsh, M. T., Iqbal, J., Josekutty, J., Soh, J., Di, L. E., Ozaydin, E., Gunduz, M., Tarugi, P., and Hussain, M. M. (2015). Novel Abetalipoproteinemia Missense Mutation Highlights the Importance of the N-Terminal beta-Barrel in Microsomal Triglyceride Transfer Protein Function. Circ. Cardiovasc. Genet. 8, 677-687.
Zhang et al., J Control Release. 2013, 172(3):962-74.

APPENDIX A
HOMO SAPIENS APOLIPOPROTEIN B (APOB), MRNA
(GENE ACCESSION NM_000384)

| | |
|---|---|
| 1 | ATTCCCACCG GGACCTGCGG GGCTGAGTGC CCTTCTCGGT TGCTGCCGCT GAGGAGCCCG |
| 61 | CCCAGCCAGC CAGGGCCGCG AGGCCGAGGC CAGGCCGCAG CCCAGGAGCC GCCCCACCGC |
| 121 | AGCTGGCGAT GGACCCGCCG AGGCCCGCGC TGCTGGCGCT GCTGGCGCTG CCTGCGCTGC |
| 181 | TGCTGCTGCT GCTGGCGGGC GCCAGGGCCG AAGAGGAAAT GCTGGAAAAT GTCAGCCTGG |
| 241 | TCTGTCCAAA AGATGCGACC CGATTCAAGC ACCTCCGGAA GTACACATAC AACTATGAGG |
| 301 | CTGAGAGTTC CAGTGGAGTC CCTGGGACTG CTGATTCAAG AAGTGCCACC AGGATCAACT |
| 361 | GCAAGGTTGA GCTGGAGGTT CCCCAGCTCT GCAGCTTCAT CCTGAAGACC AGCCAGTGCA |
| 421 | CCCTGAAAGA GGTGTATGGC TTCAACCCTG AGGGCAAAGC CTTGCTGAAG AAAACCAAGA |
| 481 | ACTCTGAGGA GTTTGCTGCA GCCATGTCCA GGTATGAGCT CAAGCTGGCC ATTCCAGAAG |
| 541 | GGAAGCAGGT TTTCCTTTAC CCGGAGAAAG ATGAACCTAC TTACATCCTG AACATCAAGA |
| 601 | GGGGCATCAT TTCTGCCCTC CTGGTTCCCC CAGAGACAGA AGAAGCCAAG CAAGTGTTGT |
| 661 | TTCTGGATAC CGTGTATGGA AACTGCTCCA CTCACTTTAC CGTCAAGACG AGGAAGGGCA |
| 721 | ATGTGGCAAC AGAAATATCC ACTGAAAGAG ACCTGGGGCA GTGTGATCGC TTCAAGCCCA |
| 781 | TCCGCACAGG CATCAGCCCA CTTGCTCTCA TCAAAGGCAT GACCCGCCCC TTGTCAACTC |
| 841 | TGATCAGCAG CAGCCAGTCC TGTCAGTACA CACTGGACGC TAAGAGGAAG CATGTGGCAG |
| 901 | AAGCCATCTG CAAGGAGCAA CACCTCTTCC TGCCTTTCTC CTACAAGAAT AAGTATGGGA |
| 961 | TGGTAGCACA AGTGACACAG ACTTTGAAAC TTGAAGACAC ACCAAAGATC AACAGCCGCT |
| 1021 | TCTTTGGTGA AGGTACTAAG AAGATGGGCC TCGCATTTGA GAGCACCAAA TCCACATCAC |
| 1081 | CTCCAAAGCA GGCCGAAGCT GTTTTGAAGA CTCTCCAGGA ACTGAAAAAA CTAACCATCT |
| 1141 | CTGAGCAAAA TATCCAGAGA GCTAATCTCT TCAATAAGCT GGTTACTGAG CTGAGAGGCC |
| 1201 | TCAGTGATGA AGCAGTCACA TCTCTCTTGC CACAGCTGAT TGAGGTGTCC AGCCCCATCA |
| 1261 | CTTTACAAGC CTTGGTTCAG TGTGGACAGC CTCAGTGCTC CACTCACATC CTCCAGTGGC |
| 1321 | TGAAACGTGT GCATGCCAAC CCCCTTCTGA TAGATGTGGT CACCTACCTG GTGGCCCTGA |
| 1381 | TCCCCGAGCC CTCAGCACAG CAGCTGCGAG AGATCTTCAA CATGGCGAGG GATCAGCGCA |
| 1441 | GCCGAGCCAC CTTGTATGCG CTGAGCCACG CGGTCAACAA CTATCATAAG ACAAACCCTA |
| 1501 | CAGGGACCCA GGAGCTGCTG GACATTGCTA ATTACCTGAT GGAACAGATT CAAGATGACT |
| 1561 | GCACTGGGGA TGAAGATTAC ACCTATTTGA TTCTGCGGGT CATTGGAAAT ATGGGCCAAA |
| 1621 | CCATGGAGCA GTTAACTCCA GAACTCAAGT CTTCAATCCT GAAATGTGTC CAAAGTACAA |
| 1681 | AGCCATCACT GATGATCCAG AAAGCTGCCA TCCAGGCTCT GCGGAAAATG GAGCCTAAAG |
| 1741 | ACAAGGACCA GGAGGTTCTT CTTCAGACTT TCCTTGATGA TGCTTCTCCG GGAGATAAGC |
| 1801 | GACTGGCTGC CTATCTTATG TTGATGAGGA GTCCTTCACA GGCAGATATT AACAAAATTG |
| 1861 | TCCAAATTCT ACCATGGGAA CAGAATGAGC AAGTGAAGAA CTTTGTGGCT TCCCATATTG |
| 1921 | CCAATATCTT GAACTCAGAA GAATTGGATA TCCAAGATCT GAAAAAGTTA GTGAAAGAAG |
| 1981 | CTCTGAAAGA ATCTCAACTT CCAACTGTCA TGGACTTCAG AAAATTCTCT CGGAACTATC |
| 2041 | AACTCTACAA ATCTGTTTCT CTTCCATCAC TTGACCCAGC CTCAGCCAAA ATAGAAGGGA |
| 2101 | ATCTTATATT TGATCCAAAT AACTACCTTC CTAAAGAAAG CATGCTGAAA ACTACCCTCA |
| 2161 | CTGCCTTTGG ATTTGCTTCA GCTGACCTCA TCGAGATTGG CTTGGAAGGA AAAGGCTTTG |
| 2221 | AGCCAACATT GGAAGCTCTT TTTGGGAAGC AAGGATTTTT CCCAGACAGT GTCAACAAAG |
| 2281 | CTTTGTACTG GGTTAATGGT CAAGTTCCTG ATGGTGTCTC TAAGGTCTTA GTGGACCACT |
| 2341 | TTGGCTATAC CAAAGATGAT AAACATGAGC AGGATATGGT AAATGGAATA ATGCTCAGTG |
| 2401 | TTGAGAAGCT GATTAAAGAT TTGAAATCCA AAGAAGTCCC GGAAGCCAGA GCCTACCTCC |
| 2461 | GCATCTTGGG AGAGGAGCTT GGTTTTGCCA GTCTCCATGA CCTCCAGCTC CTGGGAAAGC |
| 2521 | TGCTTCTGAT GGGTGCCCGC ACTCTGCAGG GGATCCCCCA GATGATTGGA GAGGTCATCA |
| 2581 | GGAAGGGCTC AAAGAATGAC TTTTTTCTTC ACTACATCTT CATGGAGAAT GCCTTTGAAC |
| 2641 | TCCCCACTGG AGCTGGATTA CAGTTGCAAA TATCTTCATC TGGAGTCATT GCTCCCGGAG |
| 2701 | CCAAGGCTGG AGTAAAACTG GAAGTAGCCA ACATGCAGGC TGAACTGGTG GCAAAACCCT |
| 2761 | CCGTGTCTGT GGAGTTTGTG ACAAATATGG GCATCATCAT TCCGGACTTC GCTAGGAGTG |
| 2821 | GGGTCCAGAT GAACACCAAC TTCTTCCACG AGTCGGGTCT GGAGGCTCAT GTTGCCCTAA |
| 2881 | AAGCTGGGAA GCTGAAGTTT ATCATTCCTT CCCCAAAGAG ACCAGTCAAG CTGCTCAGTG |
| 2941 | GAGGCAACAC ATTACATTTG GTCTCTACCA CCAAAACGGA GGTGATCCCA CCTCTCATTG |
| 3001 | AGAACAGGCA GTCCTGGTCA GTTTGCAAGC AAGTCTTTCC TGGCCTGAAT TACTGCACCT |
| 3061 | CAGGCGCTTA CTCCAACGCC AGCTCCACAG ACTCCGCCTC CTACTATCCG CTGACCGGGG |
| 3121 | ACACCAGATT AGAGCTGGAA CTGAGGCCTA CAGGAGAGAT TGAGCAGTAT TCTGTCAGCG |
| 3181 | CAACCTATGA GCTCCAGAGA GAGGACAGAG CCTTGGTGGA TACCCTGAAG TTTGTAACTC |
| 3241 | AAGCAGAAGG TGCGAAGCAG ACTGAGGCTA CCATGACATT CAAATATAAT CGGCAGAGTA |
| 3301 | TGACCTTGTC CAGTGAAGTC CAAATTCCGG ATTTTGATGT TGACCTCGGA ACAATCCTCA |
| 3361 | GAGTTAATGA TGAATCTACT GAGGGCAAAA CGTCTTACAG ACTCACCCTG GACATTCAGA |
| 3421 | ACAAGAAAAT TACTGAGGTC GCCCTCATGG GCCACCTAAG TTGTGACACA AAGGAAGAAA |
| 3481 | GAAAAATCAA GGGTGTTATT TCCATACCCC GTTTGCAAGC AGAAGCCAGA AGTGAGATCC |
| 3541 | TCGCCCACTG GTCGCCTGCC AAACTGCTTC TCCAAATGGA CTCATCTGCT ACAGCTTATG |
| 3601 | GCTCCACAGT TTCCAAGAGG GTGGCATGGC ATTATGATGA AGAGAAGATT GAATTTGAAT |
| 3661 | GGAACACAGG CACCAATGTA GATACCAAAA AAATGACTTC CAATTTCCCT GTGGATCTCT |
| 3721 | CCGATTATCC TAAGAGCTTG CATATGTATG CTAATAGACT CCTGGATCAC AGAGTCCCTC |
| 3781 | AAACAGACAT GACTTTCCGG CACGTGGGTT CCAAATTAAT AGTTGCAATG AGCTCATGGC |
| 3841 | TTCAGAAGGC ATCTGGGAGT CTTCCTTATA CCCAGACTTT GCAAGACCAC CTCAATAGCC |
| 3901 | TGAAGGAGTT CAACCTCCAG AACATGGGAT TGCCAGACTT CCACATCCCA GAAAACCTCT |
| 3961 | TCTTAAAAAG CGATGGCCGG GTCAAATATA CCTTGAACAA GAACAGTTTG AAAATTGAGA |
| 4021 | TTCCTTTGCC TTTTGGTGGC AAATCCTCCA GAGATCTAAA GATGTTAGAG ACTGTTAGGA |
| 4081 | CACCAGCCCT CCACTTCAAG TCTGTGGGAT TCCATCTGCC ATCTCGAGAG TTCCAAGTCC |
| 4141 | CTACTTTTAC CATTCCCAAG TTGTATCAAC TGCAAGTGCC TCTCCTGGGT GTTCTAGACC |
| 4201 | TCTCCACGAA TGTCTACAGC AACTTGTACA ACTGGTCCGC CTCCTACAGT GGTGGCAACA |
| 4261 | CCAGCACAGA CCATTTCAGC CTTCGGGCTC GTTACCACAT GAAGGCTGAC TCTGTGGTTG |
| 4321 | ACCTGCTTTC CTACAATGTG CAAGGATCTG GAGAAACAAC ATATGACCAC AAGAATACGT |
| 4381 | TCACACTATC ATGTGATGGG TCTCTACGCC ACAAATTTCT AGATTCGAAT ATCAAATTCA |
| 4441 | GTCATGTAGA AAAACTTGGA AACAACCCAG TCTCAAAAGG TTTACTAATA TTCGATGCAT |
| 4501 | CTAGTTCCTG GGGACCACAG ATGTCTGCTT CAGTTCATTT GGACTCCAAA AAGAAACAGC |
| 4561 | ATTTGTTTGT CAAAGAAGTC AAGATTGATG GGCAGTTCAG AGTCTCTTCG TTCTATGCTA |
| 4621 | AAGGCACATA TGGCCTGTCT TGTCAGAGGG ATCCTAACAC TGGCCGGCTC AATGGAGAGT |
| 4681 | CCAACCTGAG GTTTAACTCC TCCTACCTCC AAGGCACCAA CCAGATAACA GGAAGATATG |
| 4741 | AAGATGGAAC CCTCTCCCTC ACCTCCACCT CTGATCTGCA AAGTGGCATC ATTAAAAATA |
| 4801 | CTGCTTCCCT AAAGTATGAG AACTACGAGC TGACTTTAAA ATCTGACACC AATGGGAAGT |
| 4861 | ATAAGAACTT TGCCACTTCT AACAAGATGG ATATGACCTT CTCTAAGCAA AATGCACTGC |
| 4921 | TGCGTTCTGA ATATCAGGCT GATTACGAGT CATTGAGGTT CTTCAGCCTG CTTTCTGGAT |
| 4981 | CACTAAATTC CCATGGTCTT GAGTTAAATG CTGACATCTT AGGCACTGAC AAAATTAATA |
| 5041 | GTGGTGCTCA CAAGGCGACA CTAAGGATTG GCCAAGATGG AATATCTACC AGTGCAACGA |
| 5101 | CCAACTTGAA GTGTAGTCTC CTGGTGCTGG AGAATGAGCT GAATGCAGAG CTTGGCCTCT |
| 5161 | CTGGGGCATC TATGAAATTA ACAACAAATG GCCGCTTCAG GGAACACAAT GCAAAATTCA |
| 5221 | GTCTGGATGG GAAAGCCGCC CTCACAGAGC TATCACTGGG AAGTGCTTAT CAGGCCATGA |
| 5281 | TTCTGGGTGT CGACAGCAAA AACATTTTCA ACTTCAAGGT CAGTCAAGAA GGACTTAAGC |
| 5341 | TCTCAAATGA CATGATGGGC TCATATGCTG AAATGAAATT TGACCACACA AACAGTCTGA |
| 5401 | ACATTGCAGG CTTATCACTG GACTTCTCTT CAAAACTTGA CAACATTTAC AGCTCTGACA |
| 5461 | AGTTTTATAA GCAAACTGTT AATTTACAGC TACAGCCCTA TTCTCTGGTA ACTACTTTAA |
| 5521 | ACAGTGACCT GAAATACAAT GCTCTGGATC TCACCAACAA TGGGAAACTA CGGCTAGAAC |
| 5581 | CCCTGAAGCT GCATGTGGCT GGTAACCTAA AAGGAGCCTA CCAAAATAAT GAAATAAAAC |
| 5641 | ACATCTATGC CATCTCTTCT GCTGCCTTAT CAGCAAGCTA TAAAGCAGAC ACTGTTGCTA |
| 5701 | AGGTTCAGGG TGTGGAGTTT AGCCATCGGC TCAACACAGA CATCGCTGGG CTGGCTTCAG |
| 5761 | CCATTGACAT GAGCACAAAC TATAATTCAG ACTCACTGCA TTTCAGCAAT GTCTTCCGTT |
| 5821 | CTGTAATGGC CCCGTTTACC ATGACCATCG ATGCACATAC AAATGGCAAT GGGAAACTCG |
| 5881 | CTCTCTGGGG AGAACATACT GGGCAGCTGT ATAGCAAATT CCTGTTGAAA GCAGAACCTC |
| 5941 | TGGCATTTAC TTTCTCTCAT GATTACAAAG GCTCCACAAG TCATCATCTC GTGTCTAGGA |
| 6001 | AAAGCATCAG TGCAGCTCTT GAACACAAAG TCAGTGCCCT GCTTACTCCA GCTGAGCAGA |
| 6061 | CAGGCACCTG GAAACTCAAG ACCCAATTTA ACAACAATGA ATACAGCCAG GACTTGGATG |
| 6121 | CTTACAACAC TAAAGATAAA ATTGGCGTGG AGCTTACTGG ACGAACTCTG GCTGACCTAA |
| 6181 | CTCTACTAGA CTCCCCAATT AAAGTGCCAC TTTTACTCAG TGAGCCCATC AATATCATTG |
| 6241 | ATGCTTTAGA GATGAGAGAT GCCGTTGAGA AGCCCCAAGA ATTTACAATT GTTGCTTTTG |
| 6301 | TAAAGTATGA TAAAAACCAA GATGTTCACT CCATTAACCT CCCATTTTTT GAGACCTTGC |
| 6361 | AAGAATATTT TGAGAGGAAT CGACAAACCA TTATAGTTGT ACTGGAAAAC GTACAGAGAA |
| 6421 | ACCTGAAGCA CATCAATATT GATCAATTTG TAAGAAAATA CAGAGCAGCC CTGGGAAAAC |
| 6481 | TCCCACAGCA AGCTAATGAT TATCTGAATT CATTCAATTG GGAGAGACAA GTTTCACATG |
| 6541 | CCAAGGAGAA ACTGACTGCT CTCACAAAAA AGTATAGAAT TACAGAAAAT GATATACAAA |
| 6601 | TTGCATTAGA TGATGCCAAA ATCAACTTTA ATGAAAAACT ATCTCAACTG CAGACATATA |
| 6661 | TGATACAATT TGATCAGTAT ATTAAAGATA GTTATGATTT ACATGATTTG AAAATAGCTA |
| 6721 | TTGCTAATAT TATTGATGAA ATCATTGAAA AATTAAAAAG TCTTGATGAG CACTATCATA |
| 6781 | TCCGTGTAAA TTTAGTAAAA ACAATCCATG ATCTACATTT GTTTATTGAA AATATTGATT |
| 6841 | TTAACAAAAG TGGAAGTAGT ACTGCATCCT GGATTCAAAA TGTGGATACT AAGTACCAAA |
| 6901 | TCAGAATCCA GATACAAGAA AAACTGCAGC AGCTTAAGAG ACACATACAG AATATAGACA |
| 6961 | TCCAGCACCT AGCTGGAAAG TTAAAACAAC ACATTGAGGC TATTGATGTT AGAGTGCTTT |
| 7021 | TAGATCAATT GGGAACTACA ATTTCATTTG AAAGAATAAA TGACGTTCTT GAGCATGTCA |
| 7081 | AACACTTTGT TATAAATCTT ATTGGGGATT TTGAAGTAGC TGAGAAAATC AATGCCTTCA |
| 7141 | GAGCCAAAGT CCATGAGTTA ATCGAGAGGT ATGAAGTAGA CCAACAAATC CAGGTTTTAA |
| 7201 | TGGATAAATT AGTAGAGTTG GCCCACCAAT ACAAGTTGAA GGAGACTATT CAGAAGCTAA |
| 7261 | GCAATGTCCT ACAACAAGTT AAGATAAAAG ATTACTTTGA GAAATTGGTT GGATTTATTG |
| 7321 | ATGATGCTGT CAAGAAGCTT AATGAATTAT CTTTTAAAAC ATTCATTGAA GATGTTAACA |
| 7381 | AATTCCTTGA CATGTTGATA AAGAAATTAA AGTCATTTGA TTACCACCAG TTTGTAGATG |
| 7441 | AAACCAATGA CAAAATCCGT GAGGTGACTC AGAGACTCAA TGGTGAAATT CAGGCTCTGG |
| 7501 | AACTACCACA AAAAGCTGAA GCATTAAAAC TGTTTTTAGA GGAAACCAAG GCCACAGTTG |
| 7561 | CAGTGTATCT GGAAAGCCTA CAGGACACCA AAATAACCTT AATCATCAAT TGGTTACAGG |
| 7621 | AGGCTTTAAG TTCAGCATCT TTGGCTCACA TGAAGGCCAA ATTCCGAGAG ACCCTAGAAG |
| 7681 | ATACACGAGA CCGAATGTAT CAAATGGACA TTCAGCAGGA ACTTCAACGA TACCTGTCTC |
| 7741 | TGGTAGGCCA GGTTTATAGC ACACTTGTCA CCTACATTTC TGATTGGTGG ACTCTTGCTG |
| 7801 | CTAAGAACCT TACTGACTTT GCAGAGCAAT ATTCTATCCA AGATTGGGCT AAACGTATGA |
| 7861 | AAGCATTGGT AGAGCAAGGG TTCACTGTTC CTGAAATCAA GACCATCCTT GGGACCATGC |
| 7921 | CTGCCTTTGA AGTCAGTCTT CAGGCTCTTC AGAAAGCTAC CTTCCAGACA CCTGATTTTA |
| 7981 | TAGTCCCCCT AACAGATTTG AGGATTCCAT CAGTTCAGAT AAACTTCAAA GACTTAAAAA |
| 8041 | ATATAAAAAT CCCATCCAGG TTTTCCACAC CAGAATTTAC CATCCTTAAC ACCTTCCACA |
| 8101 | TTCCTTCCTT TACAATTGAC TTTGTAGAAA TGAAAGTAAA GATCATCAGA ACCATTGACC |
| 8161 | AGATGCTGAA CAGTGAGCTG CAGTGGCCCG TTCCAGATAT ATATCTCAGG GATCTGAAGG |
| 8221 | TGGAGGACAT TCCTCTAGCG AGAATCACCC TGCCAGACTT CCGTTTACCA GAAATCGCAA |
| 8281 | TTCCAGAATT CATAATCCCA ACTCTCAACC TTAATGATTT TCAAGTTCCT GACCTTCACA |
| 8341 | TACCAGAATT CCAGCTTCCC CACATCTCAC ACACAATTGA AGTACCTACT TTTGGCAAGC |
| 8401 | TATACAGTAT TCTGAAAATC CAATCTCCTC TTTTCACATT AGATGCAAAT GCTGACATAG |
| 8461 | GGAATGGAAC CACCTCAGCA AACGAAGCAG GTATCGCAGC TTCCATCACT GCCAAAGGAG |
| 8521 | AGTCCAAATT AGAAGTTCTC AATTTTGATT TTCAAGCAAA TGCACAACTC TCAAACCCTA |
| 8581 | AGATTAATCC GCTGGCTCTG AAGGAGTCAG TGAAGTTCTC CAGCAAGTAC CTGAGAACGG |
| 8641 | AGCATGGGAG TGAAATGCTG TTTTTTGGAA ATGCTATTGA GGGAAAATCA AACACAGTGG |
| 8701 | CAAGTTTACA CACAGAAAAA AATACACTGG AGCTTAGTAA TGGAGTGATT GTCAAGATAA |
| 8761 | ACAATCAGCT TACCCTGGAT AGCAACACTA AATACTTCCA CAAATTGAAC ATCCCCAAAC |
| 8821 | TGGACTTCTC TAGTCAGGCT GACCTGCGCA ACGAGATCAA GACACTGTTG AAAGCTGGCC |
| 8881 | ACATAGCATG GACTTCTTCT GGAAAAGGGT CATGGAAATG GGCCTGCCCC AGATTCTCAG |
| 8941 | ATGAGGGAAC ACATGAATCA CAAATTAGTT TCACCATAGA AGGACCCCTC ACTTCCTTTG |
| 9001 | GACTGTCCAA TAAGATCAAT AGCAAACACC TAAGAGTAAA CCAAAACTTG GTTTATGAAT |
| 9061 | CTGGCTCCCT CAACTTTTCT AAACTTGAAA TTCAATCACA AGTCGATTCC CAGCATGTGG |
| 9121 | GCCACAGTGT TCTAACTGCT AAAGGCATGG CACTGTTTGG AGAAGGGAAG GCAGAGTTTA |
| 9181 | CTGGGAGGCA TGATGCTCAT TTAAATGGAA AGGTTATTGG AACTTTGAAA AATTCTCTTT |
| 9241 | TCTTTTCAGC CCAGCCATTT GAGATCACGG CATCCACAAA CAATGAAGGG AATTTGAAAG |
| 9301 | TTCGTTTTCC ATTAAGGTTA ACAGGGAAGA TAGACTTCCT GAATAACTAT GCACTGTTTC |
| 9361 | TGAGTCCCAG TGCCCAGCAA GCAAGTTGGC AAGTAAGTGC TAGGTTCAAT CAGTATAAGT |
| 9421 | ACAACCAAAA TTTCTCTGCT GGAAACAACG AGAACATTAT GGAGGCCCAT GTAGGAATAA |
| 9481 | ATGGAGAAGC AAATCTGGAT TTCTTAAACA TTCCTTTAAC AATTCCTGAA ATGCGTCTAC |
| 9541 | CTTACACAAT AATCACAACT CCTCCACTGA AAGATTTCTC TCTATGGGAA AAAACAGGCT |
| 9601 | TGAAGGAATT CTTGAAAACG ACAAAGCAAT CATTTGATTT AAGTGTAAAA GCTCAGTATA |
| 9661 | AGAAAAACAA ACACAGGCAT TCCATCACAA ATCCTTTGGC TGTGCTTTGT GAGTTTATCA |
| 9721 | GTCAGAGCAT CAAATCCTTT GACAGGCATT TTGAAAAAAA CAGAAACAAT GCATTAGATT |
| 9781 | TTGTCACCAA ATCCTATAAT GAAACAAAAA TTAAGTTTGA TAAGTACAAA GCTGAAAAAT |
| 9841 | CTCACGACGA GCTCCCCAGG ACCTTTCAAA TTCCTGGATA CACTGTTCCA GTTGTCAATG |
| 9901 | TTGAAGTGTC TCCATTCACC ATAGAGATGT CGGCATTCGG CTATGTGTTC CCAAAAGCAG |
| 9961 | TCAGCATGCC TAGTTTCTCC ATCCTAGGTT CTGACGTCCG TGTGCCTTCA TACACATTAA |
| 10021 | TCCTGCCATC ATTAGAGCTG CCAGTCCTTC ATGTCCCTAG AAATCTCAAG CTTTCTCTTC |
| 10081 | CAGATTTCAA GGAATTGTGT ACCATAAGCC ATATTTTTAT TCCTGCCATG GGCAATATTA |
| 10141 | CCTATGATTT CTCCTTTAAA TCAAGTGTCA TCACACTGAA TACCAATGCT GAACTTTTTA |
| 10201 | ACCAGTCAGA TATTGTTGCT CATCTCCTTT CTTCATCTTC ATCTGTCATT GATGCACTGC |
| 10261 | AGTACAAATT AGAGGGCACC ACAAGATTGA CAAGAAAAAG GGGATTGAAG TTAGCCACAG |
| 10321 | CTCTGTCTCT GAGCAACAAA TTTGTGGAGG GTAGTCATAA CAGTACTGTG AGCTTAACCA |
| 10381 | CGAAAAATAT GGAAGTGTCA GTGGCAACAA CCACAAAAGC CCAAATTCCA ATTTTGAGAA |
| 10441 | TGAATTTCAA GCAAGAACTT AATGGAAATA CCAAGTCAAA ACCTACTGTC TCTTCCTCCA |
| 10501 | TGGAATTTAA GTATGATTTC AATTCTTCAA TGCTGTACTC TACCGCTAAA GGAGCAGTTG |
| 10561 | ACCACAAGCT TAGCTTGGAA AGCCTCACCT CTTACTTTTC CATTGAGTCA TCTACCAAAG |
| 10621 | GAGATGTCAA GGGTTCGGTT CTTTCTCGGG AATATTCAGG AACTATTGCT AGTGAGGCCA |
| 10681 | ACACTTACTT GAATTCCAAG AGCACACGGT CTTCAGTGAA GCTGCAGGGC ACTTCCAAAA |
| 10741 | TTGATGATAT CTGGAACCTT GAAGTAAAAG AAAATTTTGC TGGAGAAGCC ACACTCCAAC |
| 10801 | GCATATATTC CCTCTGGGAG CACAGTACGA AAAACCACTT ACAGCTAGAG GGCCTCTTTT |
| 10861 | TCACCAACGG AGAACATACA AGCAAAGCCA CCCTGGAACT CTCTCCATGG CAAATGTCAG |
| 10921 | CTCTTGTTCA GGTCCATGCA AGTCAGCCCA GTTCCTTCCA TGATTTCCCT GACCTTGGCC |
| 10981 | AGGAAGTGGC CCTGAATGCT AACACTAAGA ACCAGAAGAT CAGATGGAAA AATGAAGTCC |
| 11041 | GGATTCATTC TGGGTCTTTC CAGAGCCAGG TCGAGCTTTC CAATGACCAA GAAAAGGCAC |
| 11101 | ACCTTGACAT TGCAGGATCC TTAGAAGGAC ACCTAAGGTT CCTCAAAAAT ATCATCCTAC |
| 11161 | CAGTCTATGA CAAGAGCTTA TGGGATTTCC TAAAGCTGGA TGTAACCACC AGCATTGGTA |
| 11221 | GGAGACAGCA TCTTCGTGTT TCAACTGCCT TTGTGTACAC CAAAAACCCC AATGGCTATT |
| 11281 | CATTCTCCAT CCCTGTAAAA GTTTTGGCTG ATAAATTCAT TATTCCTGGG CTGAAACTAA |
| 11341 | ATGATCTAAA TTCAGTTCTT GTCATGCCTA CGTTCCATGT CCCATTTACA GATCTTCAGG |
| 11401 | TTCCATCGTG CAAACTTGAC TTCAGAGAAA TACAAATCTA TAAGAAGCTG AGAACTTCAT |
| 11461 | CATTTGCCCT CAACCTACCA ACACTCCCCG AGGTAAAATT CCCTGAAGTT GATGTGTTAA |
| 11521 | CAAAATATTC TCAACCAGAA GACTCCTTGA TTCCCTTTTT TGAGATAACC GTGCCTGAAT |
| 11581 | CTCAGTTAAC TGTGTCCCAG TTCACGCTTC CAAAAAGTGT TTCAGATGGC ATTGCTGCTT |
| 11641 | TGGATCTAAA TGCAGTAGCC AACAAGATCG CAGACTTTGA GTTGCCCACC ATCATCGTGC |
| 11701 | CTGAGCAGAC CATTGAGATT CCCTCCATTA AGTTCTCTGT ACCTGCTGGA ATTGTCATTC |
| 11761 | CTTCCTTTCA AGCACTGACT GCACGCTTTG AGGTAGACTC TCCCGTGTAT AATGCCACTT |
| 11821 | GGAGTGCCAG TTTGAAAAAC AAAGCAGATT ATGTTGAAAC AGTCCTGGAT TCCACATGCA |
| 11881 | GCTCAACCGT ACAGTTCCTA GAATATGAAC TAAATGTTTT GGGAACACAC AAAATCGAAG |
| 11941 | ATGGTACGTT AGCCTCTAAG ACTAAAGGAA CATTTGCACA CCGTGACTTC AGTGCAGAAT |
| 12001 | ATGAAGAAGA TGGCAAATAT GAAGGACTTC AGGAATGGGA AGGAAAAGCG CACCTCAATA |
| 12061 | TCAAAAGCCC AGCGTTCACC GATCTCCATC TGCGCTACCA GAAAGACAAG AAAGGCATCT |
| 12121 | CCACCTCAGC AGCCTCCCCA GCCGTAGGCA CCGTGGGCAT GGATATGGAT GAAGATGACG |
| 12181 | ACTTTTCTAA ATGGAACTTC TACTACAGCC CTCAGTCCTC TCCAGATAAA AAACTCACCA |
| 12241 | TATTCAAAAC TGAGTTGAGG GTCCGGGAAT CTGATGAGGA AACTCAGATC AAAGTTAATT |
| 12301 | GGGAAGAAGA GGCAGCTTCT GGCTTGCTAA CCTCTCTGAA AGACAACGTG CCCAAGGCCA |
| 12361 | CAGGGGTCCT TTATGATTAT GTCAACAAGT ACCACTGGGA ACACACAGGG CTCACCCTGA |
| 12421 | GAGAAGTGTC TTCAAAGCTG AGAAGAAATC TGCAGAACAA TGCTGAGTGG GTTTATCAAG |
| 12481 | GGGCCATTAG GCAAATTGAT GATATCGACG TGAGGTTCCA GAAAGCAGCC AGTGGCACCA |
| 12541 | CTGGGACCTA CCAAGAGTGG AAGGACAAGG CCCAGAATCT GTACCAGGAA CTGTTGACTC |
| 12601 | AGGAAGGCCA AGCCAGTTTC CAGGGACTCA AGGATAACGT GTTTGATGGC TTGGTACGAG |
| 12661 | TTACTCAAGA ATTCCATATG AAAGTCAAGC ATCTGATTGA CTCACTCATT GATTTTCTGA |
| 12721 | ACTTCCCCAG ATTCCAGTTT CCGGGGAAAC CTGGGATATA CACTAGGGAG GAACTTTGCA |
| 12781 | CTATGTTCAT AAGGGAGGTA GGGACGGTAC TGTCCCAGGT ATATTCGAAA GTCCATAATG |
| 12841 | GTTCAGAAAT ACTGTTTTCC TATTTCCAAG ACCTAGTGAT TACACTTCCT TTCGAGTTAA |
| 12901 | GGAAACATAA ACTAATAGAT GTAATCTCGA TGTATAGGGA ACTGTTGAAA GATTTATCAA |
| 12961 | AAGAAGCCCA AGAGGTATTT AAAGCCATTC AGTCTCTCAA GACCACAGAG GTGCTACGTA |
| 13021 | ATCTTCAGGA CCTTTTACAA TTCATTTTCC AACTAATAGA AGATAACATT AAACAGCTGA |
| 13081 | AAGAGATGAA ATTTACTTAT CTTATTAATT ATATCCAAGA TGAGATCAAC ACAATCTTCA |
| 13141 | GTGATTATAT CCCATATGTT TTTAAATTGT TGAAAGAAAA CCTATGCCTT AATCTTCATA |
| 13201 | AGTTCAATGA ATTTATTCAA AACGAGCTTC AGGAAGCTTC TCAAGAGTTA CAGCAGATCC |
| 13261 | ATCAATACAT TATGGCCCTT CGTGAAGAAT ATTTTGATCC AAGTATAGTT GGCTGGACAG |
| 13321 | TGAAATATTA TGAACTTGAA GAAAAGATAG TCAGTCTGAT CAAGAACCTG TTAGTTGCTC |
| 13381 | TTAAGGACTT CCATTCTGAA TATATTGTCA GTGCCTCTAA CTTTACTTCC CAACTCTCAA |
| 13441 | GTCAAGTTGA GCAATTTCTG CACAGAAATA TTCAGGAATA TCTTAGCATC CTTACCGATC |
| 13501 | CAGATGGAAA AGGGAAAGAG AAGATTGCAG AGCTTTCTGC CACTGCTCAG GAAATAATTA |
| 13561 | AAAGCCAGGC CATTGCGACG AAGAAAATAA TTTCTGATTA CCACCAGCAG TTTAGATATA |
| 13621 | AACTGCAAGA TTTTTCAGAC CAACTCTCTG ATTACTATGA AAAATTTATT GCTGAATCCA |
| 13681 | AAAGATTGAT TGACCTGTCC ATTCAAAACT ACCACACATT TCTGATATAC ATCACGGAGT |
| 13741 | TACTGAAAAA GCTGCAATCA ACCACAGTCA TGAACCCCTA CATGAAGCTT GCTCCAGGAG |
| 13801 | AACTTACTAT CATCCTCTAA TTTTTTAAAA GAAATCTTCA TTTATTCTTC TTTTCCAATT |
| 13861 | GAACTTTCAC ATAGCACAGA AAAAATTCAA ACTGCCTATA TTGATAAAAC CATACAGTGA |
| 13921 | GCCAGCCTTG CAGTAGGCAG TAGACTATAA GCAGAAGCAC ATATGAACTG GACCTGCACC |
| 13981 | AAAGCTGGCA CCAGGGCTCG GAAGGTCTCT GAACTCAGAA GGATGGCATT TTTTGCAAGT |
| 14041 | TAAAGAAAAT CAGGATCTGA GTTATTTTGC TAAACTTGGG GGAGGAGGAA CAAATAAATG |
| 14101 | GAGTCTTTAT TGTGTATCAT A |

HOMO SAPIENS NUCLEAR RECEPTOR
INTERACTING PROTEIN 1 (NRIP1), MRNA (GENE ACCESSION NM_003489)

| | |
|---|---|
| 1 | GCAGGCGCCT TCGCGGACCG AGCCTGACGG AGCCGGAGGC TGGGAGCCGC GGCGGCCTGG |
| 61 | GGAAGTGTTT GGATTGTGAG CTATTTCAGA ACTGTTCTCA GGACTCATTA TTTTAACATT |
| 121 | TGGGAGAAAC ACAGCCAGAA GATGCACACT TGACTGAAGG AGGACAGGGA ATCTGAAGAC |
| 181 | TCCGGATGAC ATCAGAGCTA CTTTTCAACA GCCTTCTCAA TTTTCTTTCT CAGAAAGCAG |
| 241 | AGGCTCAGAG CTTGGAGACA GACGAACACT GATATTTGCA TTTAATGGGG AACAAAAGAT |
| 301 | GAAGAAGGAA AAGGAATATA TTCACTAAGG ATTCTATCTG CTTACTGCTA CAGACCTATG |
| 361 | TGTTAAGGAA TTCTTCTCCT CCTCCTTGCG TAGAAGTTGA TCAGCACTGT GGTCAGACTG |
| 421 | CATTTATCTT GTCATTGCCA GAAGAAATCT TGGACAGAAT GTAACAGTAC GTCTCTCTCT |
| 481 | GATTGCGATG GAAGGTGATA AACTGATACT CCTTTATTAA AGTTACATCG CACTCACCAC |
| 541 | AGAAAACCAT TCTTTAAAGT GAATAGAAAC CAAGCCCTTG TGAACACTTC TATTGAACAT |
| 601 | GACTCATGGA GAAGAGCTTG GCTCTGATGT GCACCAGGAT TCTATTGTTT TAACTTACCT |
| 661 | AGAAGGATTA CTAATGCATC AGGCAGCAGG GGGATCAGGT ACTGCCGTTG ACAAAAAGTC |
| 721 | TGCTGGGCAT AATGAAGAGG ATCAGAACTT TAACATTTCT GGCAGTGCAT TTCCCACCTG |
| 781 | TCAAAGTAAT GGTCCAGTTC TCAATACACA TACATATCAG GGGTCTGGCA TGCTGCACCT |
| 841 | CAAAAAAGCC AGACTGTTGC AGTCTTCTGA GGACTGGAAT GCAGCAAAGC GGAAGAGGCT |
| 901 | GTCTGATTCT ATCATGAATT TAAACGTAAA GAAGGAAGCT TTGCTAGCTG GCATGGTTGA |
| 961 | CAGTGTGCCT AAAGGCAAAC AGGATAGCAC ATTACTGGCC TCTTTGCTTC AGTCATTCAG |
| 1021 | CTCTAGGCTG CAGACTGTTG CTCTGTCACA ACAAATCAGG CAGAGCCTCA AGGAGCAAGG |
| 1081 | ATATGCCCTC AGTCATGATT CTTTAAAAGT GGAGAAGGAT TTAAGGTGCT ATGGTGTTGC |
| 1141 | ATCAAGTCAC TTAAAAACTT TGTTGAAGAA AAGTAAAGTT AAAGATCAAA AGCCTGATAC |
| 1201 | GAATCTTCCT GATGTGACTA AAAACCTCAT CAGAGATAGG TTTGCAGAGT CTCCTCATCA |
| 1261 | TGTTGGACAA AGTGGAACAA AGGTCATGAG TGAACCGTTG TCATGTGCTG CAAGATTACA |
| 1321 | GGCTGTTGCA AGCATGGTGG AAAAAAGGGC TAGTCCTGCC ACCTCACCTA AACCTAGTGT |
| 1381 | TGCTTGTAGC CAGTTAGCAT TACTTCTGTC AAGCGAAGCC CATTTGCAGC AGTATTCTCG |
| 1441 | AGAACACGCT TTAAAAACGC AAAATGCAAA TCAAGCAGCA AGTGAAAGAC TTGCTGCTAT |
| 1501 | GGCCAGATTG CAAGAAAATG GCCAGAAGGA TGTTGGCAGT TACCAGCTCC CAAAAGGAAT |
| 1561 | GTCAAGCCAT CTTAATGGTC AGGCAAGAAC ATCATCAAGC AAACTGATGG CTAGCAAAAG |
| 1621 | TAGTGCTACA GTGTTTCAAA ATCCAATGGG TATCATTCCT TCTTCCCCTA AAAATGCAGG |
| 1681 | TTATAAGAAC TCACTGGAAA GAAACAATAT AAAACAAGCT GCTAACAATA GTTTGCTTTT |
| 1741 | ACATCTTCTT AAAAGCCAGA CTATACCTAA GCCAATGAAT GGACACAGTC ACAGTGAGAG |
| 1801 | AGGAAGCATT TTTGAGGAAA GTAGTACACC TACAACTATT GATGAATATT CAGATAACAA |
| 1861 | TCCTAGTTTT ACAGATGACA GCAGTGGTGA TGAAAGTTCT TATTCCAACT GTGTTCCCAT |
| 1921 | AGACTTGTCT TGCAAACACC GAACTGAAAA ATCAGAATCT GACCAACCTG TTTCCCTGGA |
| 1981 | TAACTTCACT CAATCCTTGC TAAACACTTG GGATCCAAAA GTCCCAGATG TAGATATCAA |
| 2041 | AGAAGATCAA GATACCTCAA AGAATTCTAA GCTAAACTCA CACCAGAAAG TAACACTTCT |
| 2101 | TCAATTGCTA CTTGGCCATA AGAATGAAGA AAATGTAGAA AAAAACACCA GCCCTCAGGG |
| 2161 | AGTACACAAT GATGTGAGCA AGTTCAATAC ACAAAATTAT GCAAGGACTT CTGTGATAGA |
| 2221 | AAGCCCCAGT ACAAATCGGA CTACTCCAGT GAGCACTCCA CCTTTACTTA CATCAAGCAA |
| 2281 | AGCAGGGTCT CCCATCAATC TCTCTCAACA CTCTCTGGTC ATCAAATGGA ATTCCCCACC |
| 2341 | ATATGTCTGC AGTACTCAGT CTGAAAAGCT AACAAATACT GCATCTAACC ACTCAATGGA |
| 2401 | CCTTACAAAA AGCAAAGACC CACCAGGAGA GAAACCAGCC CAAAATGAAG GTGCACAGAA |
| 2461 | CTCTGCAACG TTTAGTGCCA GTAAGCTGTT ACAAAATTTA GCACAATGTG GAATGCAGTC |
| 2521 | ATCCATGTCA GTGGAAGAGC AGAGACCCAG CAAACAGCTG TTAACTGGAA ACACAGATAA |
| 2581 | ACCGATAGGT ATGATTGATA GATTAAATAG CCCTTTGCTC TCAAATAAAA CAAATGCAGT |
| 2641 | TGAAGAAAAT AAAGCATTTA GTAGTCAACC AACAGGTCCT GAACCAGGGC TTTCTGGTTC |
| 2701 | TGAAATAGAA AATCTGCTTG AAAGACGTAC TGTCCTCCAG TTGCTCCTGG GGAACCCCAA |
| 2761 | CAAAGGGAAG AGTGAAAAAA AAGAGAAAAC TCCCTTAAGA GATGAAAGTA CTCAGGAACA |
| 2821 | CTCAGAGAGA GCTTTAAGTG AACAAATACT GATGGTGAAA ATAAAATCTG AGCCTTGTGA |
| 2881 | TGACTTACAA ATTCCTAACA CAAATGTGCA CTTGAGCCAT GATGCTAAGA GTGCCCCATT |
| 2941 | CTTGGGTATG GCTCCTGCTG TGCAGAGAAG CGCACCTGCC TTACCAGTGT CCGAAGACTT |
| 3001 | TAAATCGGAG CCTGTTTCAC CTCAGGATTT TTCTTTCTCC AAGAATGGTC TGCTAAGTCG |
| 3061 | ATTGCTAAGA CAAAATCAAG ATAGTTACCT GGCAGATGAT TCAGACAGGA GTCACAGAAA |
| 3121 | TAATGAAATG GCACTTCTAG AATCAAAGAA TCTTTGCATG GTCCCTAAGA AAAGGAAGCT |
| 3181 | TTATACTGAG CCATTAGAAA ATCCATTTAA AAAGATGAAA AACAACATTG TTGATGCTGC |
| 3241 | AAACAATCAC AGTGCCCCAG AAGTACTGTA TGGGTCCTTG CTTAACCAGG AAGAGCTGAA |
| 3301 | ATTTAGCAGA AATGATCTTG AATTTAAATA TCCTGCTGGT CATGGCTCAG CCAGCGAAAG |
| 3361 | TGAACACAGG AGTTGGGCCA GAGAGAGCAA AAGCTTTAAT GTTCTGAAAC AGCTGCTTCT |
| 3421 | CTCAGAAAAC TGTGTGCGAG ATTTGTCCCC GCACAGAAGT AACTCTGTGG CTGACAGTAA |
| 3481 | AAAGAAAGGA CACAAAAATA ATGTGACCAA CAGCAAACCT GAATTTAGCA TTTCTTCTTT |
| 3541 | AAATGGACTG ATGTACAGTT CCACTCAGCC CAGCAGTTGC ATGGATAACA GGACATTTTC |
| 3601 | ATACCCAGGT GTAGTAAAAA CTCCTGTGAG TCCTACTTTC CCTGAGCACT TGGGCTGTGC |
| 3661 | AGGGTCTAGA CCAGAATCTG GGCTTTTGAA TGGGTGTTCC ATGCCCAGTG AGAAAGGACC |
| 3721 | CATTAAGTGG GTTATCACTG ATGCGGAGAA GAATGAGTAT GAAAAAGACT CTCCAAGATT |
| 3781 | GACCAAAACC AACCCAATAC TATATTACAT GCTTCAAAAA GGAGGCAATT CTGTTACCAG |
| 3841 | TCGAGAAACA CAAGACAAGG ACATTTGGAG GGAGGCTTCA TCTGCTGAAA GTGTCTCACA |
| 3901 | GGTCACAGCC AAAGAAGAGT TACTTCCTAC TGCAGAAACG AAAGCTTCTT TCTTTAATTT |
| 3961 | AAGAAGCCCT TACAATAGCC ATATGGGAAA TAATGCTTCT CGCCCACACA GCGCAAATGG |
| 4021 | AGAAGTTTAT GGACTTCTGG GAAGCGTGCT AACGATAAAG AAAGAATCAG AATAAAATGT |
| 4081 | ACCTGCCATC CAGTTTTGGA TCTTTTTAAA ACTAATGAGT ATGAACTTGA GATCTGTATA |
| 4141 | AATAAGAGCA TGATTTGAAA AAAAGCATGG TATAATTGAA ACTTTTTTCA TTTTGAAAAG |
| 4201 | TATTGGTTAC TGGTGATGTT GAAATATGCA TACTAATTTT TGCTTAACAT TAGATGTCAT |
| 4261 | GAGGAAACTA CTGAACTAGC AATTGGTTGT TTAACACTTC TGTATGCATC AGATAACAAC |
| 4321 | TGTGAGTAGC CTATGAATGA AATTCTTTTA TAAATATTAG GCATAAATTA AAATGTAAAA |
| 4381 | CTCCATTCAT AGTGGATTAA TGCATTTTGC TGCCTTTATT AGGGTACTTT ATTTTGCTTT |
| 4441 | TCAGAAGTCA GCCTACATAA CACATTTTTA AAGTCTAAAC TGTTAAACAA CTCTTTAAAG |
| 4501 | GATAATTATC CAATAAAAAA AAACCTAGTG CTGATTCACA GCTTATTATC CAATTCAAAA |
| 4561 | ATAAATTAGA AAAATATATG CTTACATTTT TCACTTTTGC TAAAAAGAAA AAAAAAAGGT |
| 4621 | GTTTATTTTT AACTCTTGGA AGAGGTTTTG TGGTTCCCAA TGTGTCTGTC CCACCCTGAT |
| 4681 | CCTTTTCAAT ATATATTTCT TTAAACCTTG TGCTACTTAG TAAAAATTGA TTACAATTGA |
| 4741 | GGGAAGTTTG ATAGATCCTT TAAAAAAAAG GCAGATTTCC ATTTTTTGTA TTTTAACTAC |
| 4801 | TTTACTAAAT TAATACTCCT CCTTTTACAG AATTAGAAAA GTTAACATTT ATCTTTAGGT |
| 4861 | GGTTTCCTGA AAAGTTGAAT ATTTAAGAAA TTGTTTTTAA CAGAAGCAAA ATGGCTTTTC |
| 4921 | TTTGGACAGT TTTCACCATC TCTTGTAAAA GTTAATTCTC ACCATTCCTG TGGTACCTGC |
| 4981 | GAGTGTTATG ACCAGGATTC CTTAAACCTG AACTCAGACC ACTTGCATTA GAACCATCTG |
| 5041 | GAGCACTTGT TTTAAAATGC AGATTCATAG GCAGCATCTC AGATCTACAG AACAAGAATC |
| 5101 | TCTGCTAAGT GGACCTGGAA TCTTCCATCT GCATCTTAAC ATGCTCTCTA GGTGTTTCTT |
| 5161 | GTGTTTGAGA ACCATGACTT ATGACTTTCC TCAGAACATG AGACTGTAAA ACAAAAACAA |
| 5221 | AAAACTATGT GATGCCTCTA TTTTCCCCAA TACAGTCACA CATCAGCTCA AAATTTGCAA |
| 5281 | TATTGTAGTT CATATATTAC CGTTATGTCT TTGGAAATCG GGTTCAGAAC ACTTTTTATG |
| 5341 | ACAAAAATTG GGTGGAGGGG ATAACTTTCA TATCTGGCTC AACATCTCAG GAAAATCTGT |
| 5401 | GATTATTTGT GTGTTCTAAT GAGTAACATC TACTTAGTTA GCCTTAGGGA TGGAAAAACA |
| 5461 | GGGCCACTTA CCAAACTCAG GTGATTCCAG GATGGTTTGG AAACTTCTCC TGAATGCATC |
| 5521 | CTTAACCTTT ATTAAAACCA TTGTCCTAAG AACAATGCCA ACAAAGCTTA CAACATTTAG |
| 5581 | TTTAAACCCA AGAAGGGCAC TAAACTCAGA TTGACTAAAT AAAAAGTACA AAGGGCACAT |
| 5641 | ATACGTGACA GAATTGTACA CAATCACTCC ATTGGATCTT TTACTTTAAA GTAGTGATGA |
| 5701 | AAAGTACATG TTGATACTGT CTTAGAAGAA ATTAATATAT TAGTGAAGCC ACATGGGGTT |
| 5761 | TCAGTTGCGA AACAGGTCTG TTTTTATGTT CAGTTTGTAC AATCCACAAT TCATTCACCA |
| 5821 | GATATTTTGT TCTTAATTGT GAACCAGGTT AGCAAATGAC CTATCAAAAA TTATTCTATA |
| 5881 | ATCACTACTA GTTAGGATAT TGATTTAAAA TTGTTCTACT TGAAGTGGTT TCTAAGATTT |
| 5941 | TTATATTAAA AATAGGTGTG ATTTCCTAAT ATGATCTAAA ACCCTAAATG GTTATTTTTC |
| 6001 | CTCAGAATGA TTTGTAAATA GCTACTGGAA ATATTATACA GTAATAGGAG TGGGTATTAT |
| 6061 | GCAACATCAT GGAGAAGTGA AGGCATAGGC TTATTCTGAC ATAAAATTCC ACTGGCCAGT |
| 6121 | TGAATATATT CTATTCCATG TCCATACTAT GACAATCTTA TTGTCAACAC TATATAAATA |
| 6181 | AGCTTTTAAA CAAGTCATTT TTCTTGATCG TTGTGGAAGG TTTGGAGCCT TAGAGGTATG |
| 6241 | TCAGAAAAAA TATGTTGGTA TTCTCCCTTG GGTAGGGGGA AATGACCTTT TTACAAGAGA |
| 6301 | GTGAAATTTA GGTCAGGGAA AAGACCAAGG GCCAGCATTG CTACTTTTGT GTGTGTGTGT |
| 6361 | GTGGGTTTTG TTTTGTTTTT TTGGTTGGCT GGTTGTTTTC GTTGTTGTTA ACAAAGGAAT |
| 6421 | GAGAATATGT AATACTTAAA TAAACATGAC CACGAAGAAT GCTGTTCTGA TTTACTAGAG |
| 6481 | AATGTTCCCA ATTTGAATTT AGGGTGATTT TAAAGAACAG TGAGAAAGGG CATACATCCA |
| 6541 | CAGATTCACT TTGTTTATGC ATATGTAGAT ACAAGGATGC ACATATACAC ATTTTCAAGG |
| 6601 | ACTATTTTAG ATATCTAGAC AATTTCTTCT AATAAAGTCA TTTGTGAAAG GGTACTACAG |
| 6661 | CTTATTGACA TCAGTAAGGT AGCATTCATT ACCTGTTTAT TCTCTGCTGC ATCTTACAGA |
| 6721 | AGAGTAAACT GGTGAGAGTA TATATTTTAT ATATATATAT ATATATATAT ATATAATATG |
| 6781 | TATATATATA TATATTGACT TGTTACATGA AGATGTTAAA ATCGGTTTTT AAAGGTGATG |
| 6841 | TAAATAGTGA TTTCCTTAAT GAAAAATACA TATTTTGTAT TGTTCTAATG CAACAGAAAA |
| 6901 | GCCTTTTAAT CTCTTTGGTT CCTGTATATT CCATGTATAA GTGTAAATAT AATCAGACAG |
| 6961 | GTTTAAAAGT TGTGCATGTA TGTATACAGT TGCAAGTCTG GACAAATGTA TAGAATAAAC |
| 7021 | CTTTTATTTA AGTTGTGATT ACCTGCTGCA TGAAAAGTGC ATGGGGGACC CTGTGCATCT |
| 7081 | GTGCATTTGG CAAAATGTCT TAACAAATCA GATCAGATGT TCATCCTAAC ATGACAGTAT |
| 7141 | TCCATTTCTG GACATGACGT CTGTGGTTTA AGCTTTGTGA AAGAATGTGC TTTGATTCGA |
| 7201 | AGGGTCTTAA AGAATTTTTT TAATCGTCAA CCACTTTTAA ACATAAAGAA TTCACACAAC |
| 7261 | TACTTTCATG AATTTTTTAA TCCCATTGCA AACATTATTC CAAGAGTATC CCAGTATTAG |
| 7321 | CAATACTGGA ATATAGGCAC ATTACCATTC ATAGTAAGAA TTCTGGTGTT TACACAACCA |
| 7381 | AATTTGATGC GATCTGCTCA GTAATATAAT TTGCCATTTT TATTAGAAAT TTAATTTCTT |
| 7441 | CATGTGATGT CATGAAACTG TACATACTGC AGTGTGAATT TTTTTGTTTT GTTTTTTAAT |
| 7501 | CTTTTAGTGT TTACTTCCTG CAGTGAATTT GAATAAATGA GAAAAAATGC ATTGTC |

HOMO SAPIENS B-CELL CLL/LYMPHOMA 11B (BCL11B),
TRANSCRIPT VARIANT 2, MRNA (GENE ACCESSION NM_022898)

| | |
|---|---|
| 1 | TGCGCTTTCC ACCTACCAGA CCCTGAAAGA AAGTGTCAGG AGCCGGTGCA AAACCCAGTT |
| 61 | TAAGTTCAAG AAGACATTTG CAAGTGCAAG AGGCCAAGCA GTTTGAAGAA GTGTAAGAGA |
| 121 | TTTTTTTTCC TTCGAAAGAA TATATTTTTA AAGAAACCAG CCAGTCCGCG GAAAGCAACA |
| 181 | GCAGTTTTTT TTTTTTTTGC CTCTTTTTCT TATTTTAGAT CGAGAGGTTT TTCTTGCTTT |
| 241 | TCTTCCCTTT TTTTTCTTTT TGCAAACAAA ACAAAAAACA GCATAGAAGA AAGAGCAAAA |
| 301 | TAAAGAAGAA GAAGAGGAGG AAGAGAGGGA AAGAGAGGAA GGGAAAAAAA ACACCAACCC |
| 361 | GGGCAGAGGA GGAGGTGCGG CGGCGGCGGC GGCGGCGGCA GCGGCGGCAG CGGCGCGGCG |
| 421 | GCGGCTCGGA CCCCCTCCCC CGGCTCCCCC CATCAGTGCA GCTCTCCGGG CGATGCCAGA |
| 481 | ATAGATGCCG GGGCAATGTC CCGCCGCAAA CAGGGCAACC CGCAGCACTT GTCCCAGAGG |
| 541 | GAGCTCATCA CCCCAGAGGC TGACCATGTG GAGGCCGCCA TCCTCGAAGA AGACGAGGGT |
| 601 | CTGGAGATAG AGGAGCCAAG TGGCCTGGGG CTGATGGTGG GTGGCCCCGA CCCTGACCTG |
| 661 | CTCACCTGTG GCCAGTGTCA AATGAACTTC CCCTTGGGGG ACATCCTGGT TTTTATAGAG |
| 721 | CACAAAAGGA AGCAGTGTGG CGGCAGCTTG GGTGCCTGCT ATGACAAGGC CCTGGACAAG |
| 781 | GACAGCCCGC CACCCTCCTC ACGCTCCGAG CTCAGGAAAG TGTCCGAGCC GGTGGAGATC |
| 841 | GGGATCCAAG TCACCCCCGA CGAAGATGAC CACCTGCTCT CACCCACGAA AGGCATCTGT |
| 901 | CCCAAGCAGG AGAACATTGC AGGTAAAGAT GAGCCTTCCA GCTACATTTG CACAACATGC |
| 961 | AAGCAGCCCT TCAACAGCGC GTGGTTCCTG CTGCAGCACG CGCAGAACAC GCACGGCTTC |
| 1021 | CGCATCTACC TGGAGCCCGG GCCGGCCAGC AGCTCGCTCA CGCCGCGGCT CACCATCCCG |
| 1081 | CCGCCGCTCG GGCCGGAGGC CGTGGCGCAG TCCCCGCTCA TGAATTTCCT GGGCGACAGC |
| 1141 | AACCCCTTCA ACCTGCTGCG CATGACGGGC CCCATCCTGC GGGACCACCC GGGCTTCGGC |
| 1201 | GAGGGCCGCC TGCCGGGCAC GCCGCCTCTC TTCAGTCCCC CGCCGCGCCA CCACCTGGAC |
| 1261 | CCGCACCGCC TCAGTGCCGA GGAGATGGGG CTCGTCGCCC AGCACCCCAG TGCCTTCGAC |
| 1321 | CGAGTCATGC GCCTGAACCC CATGGCCATC GACTCGCCCG CCATGGACTT CTCGCGGCGG |
| 1381 | CTCCGCGAGC TGGCGGGCAA CAGCTCCACG CCGCCGCCCG TGTCCCCGGG CCGCGGCAAC |
| 1441 | CCTATGCACC GGCTCCTGAA CCCCTTCCAG CCCAGCCCCA AGTCCCCGTT CCTGAGCACG |
| 1501 | CCGCCGCTGC CGCCCATGCC CCCTGGCGGC ACGCCGCCCC CGCAGCCGCC AGCCAAGAGC |
| 1561 | AAGTCGTGCG AGTTCTGCGG CAAGACCTTC AAGTTCCAGA GCAATCTCAT CGTGCACCGG |
| 1621 | CGCAGTCACA CGGGCGAGAA GCCCTACAAG TGCCAGCTGT GCGACCACGC GTGCTCGCAG |
| 1681 | GCCAGCAAGC TCAAGCGCCA CATGAAGACG CACATGCACA AGGCCGGCTC GCTGGCCGGC |
| 1741 | CGCTCCGACG ACGGGCTCTC GGCCGCCAGC TCCCCCGAGC CCGGCACCAG CGAGCTGGCG |
| 1801 | GGCGAGGGCC TCAAGGCGGC CGACGGTGAC TTCCGCCACC ACGAGAGCGA CCCGTCGCTG |
| 1861 | GGCCACGAGC CGGAGGAGGA GGACGAGGAG GAGGAGGAGG AGGAGGAGGA GCTGCTACTG |
| 1921 | GAGAACGAGA GCCGGCCCGA GTCGAGCTTC AGCATGGACT CGGAGCTGAG CCGCAACCGC |
| 1981 | GAGAACGGCG GTGGTGGGGT GCCCGGGGTC CCGGGCGCGG GGGGCGGCGC GGCCAAGGCG |
| 2041 | CTGGCTGACG AGAAGGCGCT GGTGCTGGGC AAGGTCATGG AGAACGTGGG CCTAGGCGCA |
| 2101 | CTGCCGCAGT ACGGCGAGCT CCTGGCCGAC AAGCAGAAGC GCGGCGCCTT CCTGAAGCGT |
| 2161 | GCGGCGGGCG GCGGGGACGC GGGCGACGAC GACGACGCGG GCGGCTGCGG GGACGCGGGC |
| 2221 | GCGGGCGGCG CGGTCAACGG GCGCGGGGGC GGCTTCGCGC CAGGCACCGA GCCCTTCCCC |
| 2281 | GGGCTCTTCC CGCGCAAGCC CGCGCCGCTG CCCAGCCCCG GGCTCAACAG CGCCGCCAAG |
| 2341 | CGCATCAAGG TGGAGAAGGA CCTGGAGCTG CCGCCCGCCG CGCTCATCCC GTCCGAGAAC |
| 2401 | GTGTACTCGC AGTGGCTGGT GGGCTACGCG GCGTCGCGGC ACTTCATGAA GGACCCCTTC |
| 2461 | CTGGGCTTCA CGGACGCACG ACAGTCGCCC TTCGCCACGT CGTCCGAGCA CTCGTCCGAG |
| 2521 | AACGGCAGCC TGCGCTTCTC CACGCCGCCC GGGGACCTGC TGGACGGCGG CCTCTCGGGC |
| 2581 | CGCAGCGGCA CGGCCAGCGG AGGCAGCACC CCGCACCTGG GCGGCCCGGG CCCCGGGCGG |
| 2641 | CCCAGCTCCA AGGAGGGCCG CCGCAGCGAC ACGTGCGAGT ACTGCGGCAA GGTGTTCAAG |
| 2701 | AACTGCAGCA ACTTGACGGT GCACCGGCGG AGCCACACCG GCGAGCGGCC TTACAAGTGC |
| 2761 | GAGCTGTGCA ACTACGCGTG CGCGCAGAGC AGCAAGCTCA CGCGCCACAT GAAGACGCAC |
| 2821 | GGGCAGATCG GCAAGGAGGT GTACCGCTGC GACATCTGCC AGATGCCCTT CAGCGTCTAC |
| 2881 | AGCACCCTGG AGAAACACAT GAAAAAGTGG CACGGCGAGC ACTTGCTGAC TAACGACGTC |
| 2941 | AAAATCGAGC AGGCCGAGAG GAGCTAAGCG CGCGGGCCCC GGCGCCCCGC ACCTGTACAG |
| 3001 | TGGAACCGTT GCCAACCGAG AGAATGCTGA CCTGACTTGC CTCCGTGTCA CCGCCACCCC |
| 3061 | GCACCCCGCG TGTCCCCGGG GCCCAGGGGA GGCGGCACTC CAACCTAACC TGTGTCTGCG |
| 3121 | AAGTCCTATG GAAACCCGAG GGTTGATTAA GGCAGTACAA ATTGTGGAGC CTTTTAACTG |
| 3181 | TGCAATAATT TCTGTATTTA TTGGGTTTTG TAATTTTTTT GGCATGTGCA GGTACTTTTT |
| 3241 | ATTATTATTT TTTCTGTTTG AATTCCTTTA AGAGATTTTG TTGGGTATCC ATCCCTTCTT |
| 3301 | TGTTTTTTTT TTAACCCGGT AGTAGCCTGA GCAATGACTC GCAAGCAATG TTAGAGGGGA |
| 3361 | AGCATATCTT TTAAATTATA ATTTGGGGGG AGGGGTGGTG CTGCTTTTTT GAAATTTAAG |
| 3421 | CTAAGCATGT GTAATTTCTT GTGAAGAAGC CAACACTCAA ATGACTTTTA AAGTTGTTTA |
| 3481 | CTTTTTCATT CCTTCCTTTT TTTTGTCCTG AAATAAAAAG TGGCATGCAG TTTTTTTTTT |
| 3541 | AATTATTTTT TAATTTTTTT TTTGGTTTTT GTTTTTGGGG TGGGGGGTGT GGATGTACAG |
| 3601 | CGGATAACAA TCTTTCAAGT CGTAGCACTT TGTTTCAGAA CTGGAATGGA GATGTAGCAC |
| 3661 | TCATGTCGTC CCGAGTCAAG CGGCCTTTTC TGTGTTGATT TCGGCTTTCA TATTACATAA |
| 3721 | GGGAAACCTT GAGTGGTGGT GCTGGGGGAG GCACCCCACA GACTCAGCGC CGCCAGAGAT |
| 3781 | AGGGTTTTTG GAGGGCTCCT CTGGGAAATG GCCCGACAGC ATTCTGAGGT TGTGCATGAC |
| 3841 | CAGCAGATAC TATCCTGTTG GTGTGCCCTG GGGTGCCATG GCTGCTATTC GCTGTAGATT |
| 3901 | AGGCTACATA AAATGGGCTG AGGGTACCTT TTTGGGGAGA TGGGGTGGCC TGCAGTGACA |
| 3961 | CAGAAAGGAA GAAACTAGCG GTGTTCTTTT AGGCGTTTTC TGGCTTGACG GCTTCTCTCT |
| 4021 | TTTTTTAAAT CACCCCCACC ACATAAATCT CAAATCCTAT GTTGCTACAA GGGGTCATCC |
| 4081 | ATCATTTCCC AAGCAGACGA ATGCCCTAAT TAATTGAAGT TAGTGTTCTC TCATTTAATG |
| 4141 | CACACTGATG ATATTGTAGG GATGGGTGGG GTGGGGATCT TGCAAATTTC TATTCTCTTT |
| 4201 | TACTGAAAAA GCAGGGGATG AGTTCCATCA GAAGGTGCCC AGCGCTACTT CCCAGGTTTT |
| 4261 | TATTTTTTTT TTCCTATCTC ATTAGGTTGG AAGGTACTAA ATATTGAACT GTTAAGATTA |
| 4321 | GACATTTGAA TTCTGTTGAC CCGCACTTTA AAGCTTTTGT TTGCATTTAA ATTAAATGGC |
| 4381 | TTCTAAACAA GAAATTGCAG CATATTCTTC TCTTTGGCCC AGAGGTGGGT TAAACTGTAA |
| 4441 | GGGACAGCTG AGATTGAGTG TCAGTATTGC TAAGCGTGGC ATTCACAATA CTGGCACTAT |
| 4501 | AAAGAACAAA ATAAAATAAT AATTTATAGG ACAGTTTTTC TACTGCCATT CAATTTGATG |
| 4561 | TGAGTGCCTT GAAAACTGAT CTTCCTATTT GAGTCTCTTG AGACAAATGC AAAACTTTTT |
| 4621 | TTTTGAAATG AAAAGACTTT TTAAAAAAGT AAAACAAGAA AAGTACATTC TTTAGAAACT |
| 4681 | AACAAAGCCA CATTTACTTT AAGTAAAAAA AAAAAAAATT CTGGTTGAAG ATAGAGGATA |
| 4741 | TGAAATGCCA TAAGACCCAA TCAAATGAAG AAATAAACCC AGCACAACCT TGGACATCCA |
| 4801 | TTAGCTGAAT TATCCTCAGC CCCTTTTGTT TTTGGGACAA CGCTGCTTAG ATATGGAGTG |
| 4861 | GAGGTGATTT ACTGCTGAAT TAAAACTCAA GTGACACAAG TTACAAGTTG ATATCGTTGA |
| 4921 | ATGAAAAGCA AAACAAAAAC AATTCAGGAA CAACGGCTAA TTTTTTCTAA AGTTAAATTT |
| 4981 | AGTGCACTCT GTCTTAAAAA TACGTTTACA GTATTGGGTA CATACAAGGG TAAAAAAAAA |
| 5041 | ATTGTGTGTA TGTGTGTTGG AGCGATCTTT TTTTTTCAAA GTTTGCTTAA TAGGTTATAC |
| 5101 | AAAAATGCCA CAGTGGCCGC GTGTATATTG TTTTCTTTTG GTGACGGGGT TTTAGTATAT |
| 5161 | ATTATATATA TTAAAATTTC TTGATTACTG TAAAAGTGGA CCAGTATTTG TAATAATCGA |
| 5221 | GAATGCCTGG GCATTTTACA AAACAAGAAA AAAAATACCC TTTTCTTTTC CTTGAAAATG |
| 5281 | TTGCAGTAAA ATTTAAATGG TGGGTCTATA AATTTGTTCT TGTTACAGTA ACTGTAAAGT |
| 5341 | CGGAGTTTTA GTAAATTTTT TTCTGCCTTG GGTGTTGAAT TTTTATTTCA AAAAAAATGT |
| 5401 | ATAGAAACTT GTATTTGGGG ATTCAAAGGG GATTGCTACA CCATGTAGAA AAAGTATGTA |
| 5461 | GAAAAAAAGT GCTTAATATT GTTATTGCTT TGCAGAAAAA AAAAAAATCA CATTTCTGAC |
| 5521 | CTGTACTTAT TTTTCTCTTC CCGCCTCCCT CTGGAATGGA TATATTGGTT GGTTCATATG |
| 5581 | ATGTAGGCAC TTGCTGTATT TTTACTGGAG CTCGTAATTT TTTAACTGTA AGCTTGTCCT |
| 5641 | TTTAAAGGGA TTTAATGTAC CTTTTTGTTA GTGAATTTGG AAATAAAAAG AAAAAAAAAA |
| 5701 | CAAAAACAAA CAGGCTGCCA TAATATATTT TTTTAATTTG GCAGGATAAA ATATTGCAAA |
| 5761 | AAAAACACAT TTGTATGTTA AGTCCTATTG TACAGGAGAA AAAGGGTTGT TTGACAACCT |
| 5821 | TTGAGAAAAA GAAACAAAAG GAAGTAGTTA AATGCTTTGG TTCACAAATC ATTTAGTTGT |
| 5881 | ATATATTTTT TGTCGGAATT GGCCTACACA GAGAACCGTT CGTGTTGGGC TTCTCTCTGA |
| 5941 | ACGCCCCGAA CCTTGCATCA AGGCTCCTTG GTGTGGCCAC AGCAGACCAG ATGGGAAATT |
| 6001 | ATTTGTGTTG AGTGGAAAAA AATCAGTTTT TGTAAAGATG TCAGTAACAT TCCACATCGT |
| 6061 | CCTCCCTTTC TCTAAGAGGC CATCTCTAAG ATGTCAGATG TAGAGGAGAG AGAGCGAGAG |
| 6121 | AACATCTTCC TTCTCTACCA TCACTCCTGT GGCGGTCACC ACCACCACCT CTCCCGCCCT |
| 6181 | TACCAGCAGA AAGCAATGCA AACTGAGCTG CTTTAGTCCT TGAGAAATTG TGAAACAAAC |
| 6241 | ACAAATATCA TAAAAGGAGC TGGTGATTCA GCTGGGTCCA GGTGAAGTGA CCTGCTGTTG |
| 6301 | AGACCGGTAC AAATTGGATT TCAGGAAGGA GACTCCATCA CAGCCAGGAC CTTTCGTGCC |
| 6361 | ATGGAGAGTG TTGGCCTCTT GTCTTTCTTC CCTGCTTTGC TGCTTTGCTC TCTGAAACCT |
| 6421 | ACATTCCGTC AGTTTCCGAA TGCGAGGGCC TGGGATGAAT TTGGTGCCTT TCCATATCTC |
| 6481 | GTTCTCTCTC CTTCCCCTGC GTTTCCTCTC CATCCTTCAT CCTCCATTGG TCCTTTTTTT |
| 6541 | TTCTTTCATT TTTTATTTAA TTTCTTTTCT TCCTGTCTGT TCCTCCCCTA ATCCTCTATT |
| 6601 | TTATTTTTAT TTTTTGTAAA GCCAAGTAGC TTTAAGATAA AGTGGTGGTC TTTTGGATGA |
| 6661 | GGGAATAATG CATTTTTAAA TAAAATACCA ATATCAGGAA GCCATTTTTT ATTTCAGGAA |
| 6721 | ATGTAAGAAA CCATTATTTC AGGTTATGAA AGTATAACCA AGCATCCTTT TGGGCAATTC |
| 6781 | CTTACCAAAT GCAGAAGCTT TTCTGTTCGA TGCACTCTTT CCTCCTTGCC ACTTACCTTT |
| 6841 | GCAAAGTTAA AAAAAAGGGG GGAGGGAATG GGAGAGAAAG CTGAGATTTC AGTTTCCTAC |
| 6901 | TGCAGTTTCC TACCTGCAGA TCCAGGGGCT GCTGTTGCCT TTGGATGCCC CACTGAGGTC |
| 6961 | CTAGAGTGCC TCCAGGGTGG TCTTCCTGTA GTCATAACAG CTAGCCAGTG CTCACCAGCT |
| 7021 | TACCAGATTG CCAGGACTAA GCCATCCCAA AGCACAAGCA TTGTGTGTCT CTGTGACTGC |
| 7081 | AGAGAAGAGA GAATTTTGCT TCTGTTTTGT GTTTAAAAAA CCAACACGGA AGCAGATGAT |
| 7141 | CCCGAGAGAG AGGCCTCTAG CATGGGTGAC CCAGCCGACC TCAGGCCGGT TTCCGCACTG |
| 7201 | CCACAACTTT GTTCAAAGTT GCCCCCAATT GGAACCTGCC ACTTGGCATT AGAGGGTCTT |
| 7261 | TCATGGGGAG AGAAGGAGAC TGAATTACTC TAAGCAAAAT GTGAAAAGTA AGGAAATCAG |
| 7321 | CCTTTCATCC CGGTCCTAAG TAACCGTCAG CCGAAGGTCT CGTGGAACAC AGGCAAACCC |
| 7381 | GTGATTTTGG TGCTCCTTGT AACTCAGCCC TGCAAAGCAA AGTCCCATTG ATTTAAGTTG |
| 7441 | TTTGCATTTG TACTGGCAAG GCAAAATATT TTTATTACCT TTTCTATTAC TTATTGTATG |
| 7501 | AGCTTTTGTT GTTTACTTGG AGGTTTTGTC TTTTACTACA AGTTTGGAAC TATTTATTAT |
| 7561 | TGCTTGGTAT TTGTGCTCTG TTTAAGAAAC AGGCACTTTT TTTTATTATG GATAAAATGT |
| 7621 | TGAGATGACA GGAGGTCATT TCAATATGGC TTAGTAAAAT ATTTATTGTT CCTTTATTCT |
| 7681 | CTGTACAAGA TTTTGGGCCT CTTTTTTTCC TTAATGTCAC AATGTTGAGT TCAGCATGTG |
| 7741 | TCTGCCATTT CATTTGTACG CTTGTTCAAA ACCAAGTTTG TTCTGGTTTC AAGTTATAAA |
| 7801 | AATAAATTGG ACATTTAACT TGATCTCCAA A |

1. A method of treating or preventing at least one of atherosclerosis, cardiovascular disease, hyperlipidemia, dyslipidemia, obesity, type II diabetes, or metabolic syndrome, comprising administering a therapeutically effective amount of a micro-RNA (miR) comprising SEQ ID NO:1 to a subject in need thereof.

2. The method of claim 1, wherein the subject is a human.

3. The method of claim 1, wherein the micro-RNA is administered at a dose of 0.1-2 mg/kg/week.

4. The method of claim 3, wherein the micro-RNA is administered at a dose of 0.1-0.5 mg/kg/week.

5. The method of claim 3, wherein the micro-RNA is administered at a dose of 0.5-1 mg/kg/week.

6. The method of claim 3, wherein the micro-RNA is administered at a dose of 1-2 mg/kg/week.

7. The method of claim 3, wherein the micro-RNA is administered at a dose of 0.1 mg/kg/week.

8. The method of claim 3, wherein the micro-RNA is administered at a dose of 1 mg/kg/week.

9. The method of claim 6, wherein the micro-RNA is administered at a dose of 1.5 mg/kg/week.

10. The method of claim 3, wherein the micro-RNA is administered at a dose of 2 mg/kg/week.

11. The method of claim 1, wherein the miRNA has at least 70%, identity to SEQ ID NO:2.

12. The method of claim 1, wherein the miRNA has at least 75%, identity to SEQ ID NO:2.

13. The method of claim 1, wherein the miRNA has at least 80%, identity to SEQ ID NO:2.

14. The method of claim 1, wherein the miRNA has at least 85%, identity to SEQ ID NO:2.

15. The method of claim 1, wherein the miRNA has at least 90%, identity to SEQ ID NO:2.

16. The method of claim 1, wherein the miRNA has at least 95%, identity to SEQ ID NO:2.

17. The method of claim 1, wherein the miRNA is miR-1200.

18. The method of claim 1, wherein apoB is decreased.

19. The method of claim 1, wherein apoAI is increased.

20. The method of claim 1, wherein NCORI is decreased.

21. The method of claim 18, wherein apoAI is increased.

22. The method of claim 18, wherein NCORI is decreased.

23. The method of claim 1, wherein LDL is decreased.

24. The method of claim 1, wherein VLDL is decreased.

25. The method of claim 1, wherein HDL is increased.

26. The method of claim 23, wherein HDL is increased.

27. The method of claim 19, wherein the miR inhibits expression of BCL11B.

28. The method of claim 1, wherein reverse cholesterol transport is increased.

29. A method of increasing apoAI expression or secretion by a cell, comprising contacting the cell with an inhibitor of BCL11B, thereby increasing expression or secretion of apoAI.

30. The method of claim 29, wherein the inhibitor is a small molecule.

31. The method of claim 29, wherein the inhibitor is a nucleic acid.

32. The method of claim 31, wherein the inhibitor is a miR.

33. The method of claim 32, wherein the miR comprises the sequence of SEQ ID NO:1.

34. The method of claim 32, wherein the miR has at least 75%, identity to SEQ ID NO:2.

35. The method of claim 32, wherein the miR has at least 80%, identity to SEQ ID NO:2.

36. The method of claim 32, wherein the miR has at least 85%, identity to SEQ ID NO:2.

37. The method of claim 32, wherein the miR has at least 90%, identity to SEQ ID NO:2.

38. The method of claim 32, wherein the miR has at least 95%, identity to SEQ ID NO:2.

39. The method of claim 32, wherein the miR is miR-1200.

40. A method of increasing HDL in a subject in need thereof, comprising administering a therapeutically effective amount of an inhibitor of BCL11B, thereby increasing HDL.

41. The method of claim 40, wherein the inhibitor is a small molecule.

42. The method of claim 40, wherein the inhibitor is a nucleic acid.

43. The method of claim 42, wherein the inhibitor is a miR.

44. The method of claim 43, wherein the miR comprises the sequence of SEQ ID NO:1.

45. The method of claim 43, wherein the miR has at least 70%, identity to SEQ ID NO:2.

46. The method of claim 43, wherein the miR has at least 75%, identity to SEQ ID NO:2.

47. The method of claim 43, wherein the miR has at least 80%, identity to SEQ ID NO:2.

48. The method of claim 43, wherein the miR has at least 85%, identity to SEQ ID NO:2.

49. The method of claim 43, wherein the miR has at least 90%, identity to SEQ ID NO:2.

50. The method of claim 43, wherein the miR has at least 95%, identity to SEQ ID NO:2.

51. The method of claim 43, wherein the miR is miR-1200.

52. The method of claim 1 or 40, wherein the route of administration is oral, nasal, buccal, sublingual, or transdermal, subcutaneous, intrasternal, intracutaneous, intramuscular, intraarticular, intraperitoneal, intrasynovial, intrathecal, intralesional, intravenous or intradermal injection or infusion.

53. The method of claim 40, wherein the subject is a human patient.

54. The method of claim 1 or 40, wherein delivery is facilitated by at least one carrier of the group consisting of a liposome, a nanoparticle, a polyurethane, a disulfide linked nanocarrier, a dendrimer, a PLGA particle, a protamine, a polymer, and a translocation domain derived peptide.

55. A micro-RNA (miR) comprising SEQ ID NO:1 for use as a medicament.

56. A miR comprising SEQ ID NO:1 for use in the treatment or prevention of at least one of atherosclerosis, cardiovascular disease, hyperlipidemia, dyslipidemia, obesity, type II diabetes, or metabolic syndrome.

57. The miR of claim 55 or 56 for use in treatment of a human.

58. The miR of claim 55 or 56, wherein the miR is administered at a dose of 0.1-2 mg/kg/week.

59. The miR of claim 55 or 56, wherein the miR is administered at a dose of 0.1-0.5 mg/kg/week.

60. The miR of claim 55 or 56, wherein the miR is administered at a dose of 0.5-1 mg/kg/week.

61. The miR of claim 55 or 56, wherein the miR is administered at a dose of 1-2 mg/kg/week.

62. The miR of claim 55 or 56, wherein the miR is administered at a dose of 0.1 mg/kg/week.

63. The miR of claim 55 or 56, wherein the miR is administered at a dose of 1 mg/kg/week.

64. The miR of claim 55 or 56, wherein the miR is administered at a dose of 1.5 mg/kg/week.

65. The miR of claim 55 or 56, wherein the miR is administered at a dose of 2 mg/kg/week.

66. The miR of claim 55 or 56, wherein the miR has at least 70%, identity to SEQ ID NO:2.

67. The miR of claim 55 or 56, wherein the miR has at least 75%, identity to SEQ ID NO:2.

68. The miR of claim 55 or 56, wherein the miR has at least 80%, identity to SEQ ID NO:2.

69. The miR of claim 55 or 56, wherein the miR has at least 85%, identity to SEQ ID NO:2.

70. The miR of claim 55 or 56, wherein the miR has at least 90%, identity to SEQ ID NO:2.

71. The miR of claim 55 or 56, wherein the miR has at least 95%, identity to SEQ ID NO:2.

72. The miR of claim 55 or 56, wherein the miR is miR-1200.

73. The miR of claim 55 or 56, wherein apoB is decreased.

74. The miR of claim 55 or 56, wherein apoAI is increased.

75. The miR of claim 55 or 56, wherein NCORI is decreased.

76. The miR of claim 73, wherein apoAI is increased.

77. The miR of claim 55 or 56, wherein NCORI is decreased.

78. The miR of claim 55 or 56, wherein LDL is decreased.

79. The miR of claim 55 or 56, wherein VLDL is decreased.

80. The miR of claim 55 or 56, wherein HDL is increased.

81. The miR of claim 78, wherein HDL is increased.

82. The miR of claim 55 or 56, wherein the miR inhibits expression of BCL11B.

83. An inhibitor of BCL11B for use as a medicament.

84. An inhibitor of BCL11B for use in the treatment or prevention of at least one of low HDL, atherosclerosis, cardiovascular disease, hyperlipidemia, dyslipidemia, obesity, type II diabetes or metabolic syndrome.

85. The inhibitor of claim 83 or 84, wherein the inhibitor comprises a small molecule.

86. The inhibitor of claim 83 or 84, wherein the inhibitor comprises a nucleic acid.

87. The inhibitor of claim 83 or 84, wherein the inhibitor comprises a miR.

88. The inhibitor of claim 83 or 84, wherein the inhibitor comprises a miR having the sequence of SEQ ID NO:1.

89. The inhibitor of claim 87, wherein the miR has at least 70%, identity to SEQ ID NO:2.

90. The inhibitor of claim 87, wherein the miR has at least 75%, identity to SEQ ID NO:2.

91. The inhibitor of claim 87, wherein the miR has at least 80%, identity to SEQ ID NO:2.

92. The inhibitor of claim 87, wherein the miR has at least 85%, identity to SEQ ID NO:2.

93. The inhibitor of claim 87, wherein the miR has at least 90%, identity to SEQ ID NO:2.

94. The inhibitor of claim 87, wherein the miR has at least 95%, identity to SEQ ID NO:2.

95. The inhibitor of claim 87, wherein the miR is miR-1200.

96. The miR of claim 87, wherein the miR is administered by oral, nasal, buccal, sublingual, or transdermal, subcutaneous, intrasternal, intracutaneous, intramuscular, intraarticular, intraperitoneal, intrasynovial, intrathecal, intralesional, intravenous or intradermal administration or injection or infusion.

97. The miR of claim 87, wherein the subject is a human patient.

98. The miR of claim 87, wherein delivery of the miR is facilitated by at least one carrier of the group consisting of a liposome, a nanoparticle, a polyurethane, a disulfide linked nanocarrier, a dendrimer, a PLGA particle, a protamine, a polymer, and a translocation domain derived peptide.

99. A method of increasing apoAI expression or secretion by a cell, comprising contacting the cell with an inhibitor of NRIP1, thereby increasing expression or secretion of apoAI.

100. The method of claim 99, wherein the inhibitor is a small molecule.

101. The method of claim 99, wherein the inhibitor is a nucleic acid.

102. The method of claim 101, wherein the inhibitor is an siRNA.

103. The method of claim 102, wherein the inhibitor is an siRNA comprising the sequence of SEQ ID NO:3.

104. The method of claim 102, wherein the siRNA has at least 75%, identity to SEQ ID NO:3.

105. The method of claim 102, wherein the siRNA has at least 80%, identity to SEQ ID NO:3.

106. The method of claim 102, wherein the siRNA has at least 85%, identity to SEQ ID NO:3.

107. The method of claim 102, wherein the siRNA has at least 90%, identity to SEQ ID NO:3.

108. The method of claim 102, wherein the siRNA has at least 95%, identity to SEQ ID NO:3.

109. The method of claim 102, wherein the siRNA is siNRIP1.

110. A method of increasing HDL in a subject in need thereof, comprising administering a therapeutically effective amount of an inhibitor of NRIP1, thereby increasing HDL.

111. The method of claim 110, wherein the inhibitor is a small molecule.

112. The method of claim 110, wherein the inhibitor is a nucleic acid.

113. The method of claim 112, wherein the inhibitor is an siRNA.

114. The method of claim 113, wherein the siRNA comprises the sequence of SEQ ID NO:3.

115. The method of claim 113, wherein the siRNA has at least 70%, identity to SEQ ID NO:3.

116. The method of claim 113, wherein the siRNA has at least 75%, identity to SEQ ID NO:3.

117. The method of claim 113, wherein the siRNA has at least 80%, identity to SEQ ID NO:3.

118. The method of claim 113, wherein the siRNA has at least 85%, identity to SEQ ID NO:3.

119. The method of claim 113, wherein the siRNA has at least 90%, identity to SEQ ID NO:3.

120. The method of claim 113, wherein the siRNA has at least 95%, identity to SEQ ID NO:3.

121. The method of claim 113, wherein the siRNA is siNRIP1.

122. The method of claim 110, wherein the route of administration is oral, nasal, buccal, sublingual, or transdermal, subcutaneous, intrasternal, intracutaneous, intramuscular, intraarticular, intraperitoneal, intrasynovial, intrathecal, intralesional, intravenous or intradermal injection or infusion.

123. The method of claim 110, wherein the subject is a human patient.

124. The method of claim 1, wherein the miR comprises SEQ ID NO:2.

125. The method of claim 124, wherein the miR consists of the sequence of SEQ ID NO:2.

126. The miR of claim 56, wherein the miR comprises the sequence of SEQ ID NO:2

127. The miR of claim 126, wherein the miR consists of the sequence of SEQ ID NO:2.

128. A method of increasing apoAI expression or secretion by a cell, comprising contacting the cell with an inhibitor of BCL11B, thereby increasing expression or secretion of apoAI.

129. The method of claim 128, wherein the inhibitor is a small molecule.

130. The method of claim 128, wherein the inhibitor is a nucleic acid.

131. The method of claim 130, wherein the inhibitor is an siRNA.

132. The method of claim 130, wherein the inhibitor is an siRNA comprising the sequence of SEQ ID NO:4.

133. The method of claim 131, wherein the siRNA has at least 75%, identity to SEQ ID NO:4.

134. The method of claim 131, wherein the siRNA has at least 80%, identity to SEQ ID NO:4.

135. The method of claim 131, wherein the siRNA has at least 85%, identity to SEQ ID NO:4.

136. The method of claim 131, wherein the siRNA has at least 90%, identity to SEQ ID NO:4.

137. The method of claim 131, wherein the siRNA has at least 95%, identity to SEQ ID NO:4.

138. The method of claim 131, wherein the siRNA is siBCL11B.

139. A method of increasing HDL in a subject in need thereof, comprising administering a therapeutically effective amount of an inhibitor of BCL11B, thereby increasing HDL.

140. The method of claim 139, wherein the inhibitor is a small molecule.

141. The method of claim 139, wherein the inhibitor is a nucleic acid.

142. The method of claim 141, wherein the inhibitor is an siRNA.

143. The method of claim 142, wherein the siRNA comprises the sequence of SEQ ID NO:4.

144. The method of claim 142, wherein the siRNA has at least 70%, identity to SEQ ID NO:4.

145. The method of claim 142, wherein the siRNA has at least 75%, identity to SEQ ID NO:4.

146. The method of claim 142, wherein the siRNA has at least 80%, identity to SEQ ID NO:4.

147. The method of claim 142, wherein the siRNA has at least 85%, identity to SEQ ID NO:4.

148. The method of claim 142, wherein the siRNA has at least 90%, identity to SEQ ID NO:4.

149. The method of claim 142, wherein the siRNA has at least 95%, identity to SEQ ID NO:4.

150. The method of claim 142, wherein the siRNA is siBCL11B.

151. The method of claim 139, wherein the route of administration is oral, nasal, buccal, sublingual, or transdermal, subcutaneous, intrasternal, intracutaneous, intramuscular, intraarticular, intraperitoneal, intrasynovial, intrathecal, intralesional, intravenous or intradermal injection or infusion.

152. The method of claim 139, wherein the subject is a human patient.


SEQUENCE LISTING (50 sequences)

SEQ ID NO: 1 - RNA, 6 nt, Artificial Sequence
  Description of Artificial Sequence Synthetic oligonucleotide
uccuga

SEQ ID NO: 2 - RNA, 22 nt, Artificial Sequence
  Description of Artificial Sequence Synthetic oligonucleotide
cuccugagccauucugagccuc

SEQ ID NO: 3 - RNA, 19 nt, Homo sapiens
gaacaaaggucaugaguga

SEQ ID NO: 4 - RNA, 19 nt, Homo sapiens
gagcaagucgugcgaguuc

SEQ ID NO: 5 - RNA, 19 nt, Homo sapiens
ucaccgcgcucauggacuu

SEQ ID NO: 6 - DNA, 24 bp, Artificial Sequence
  Description of Artificial Sequence Synthetic primer
gcagagactatgtgtcccagtttg

SEQ ID NO: 7 - DNA, 21 bp, Artificial Sequence
  Description of Artificial Sequence Synthetic primer
ccagttgtcaaggagctttag

SEQ ID NO: 8 - DNA, 20 bp, Artificial Sequence
  Description of Artificial Sequence Synthetic primer
tgaccttgtccagtgaagtc

SEQ ID NO: 9 - DNA, 20 bp, Artificial Sequence
  Description of Artificial Sequence Synthetic primer
gttctgaatgtccagggtga

SEQ ID NO: 10 - DNA, 20 bp, Artificial Sequence
  Description of Artificial Sequence Synthetic primer
tggtctccaagcagagtgtg

SEQ ID NO: 11 - DNA, 19 bp, Artificial Sequence
  Description of Artificial Sequence Synthetic primer
gagcagcagctcccaatac

SEQ ID NO: 12 - DNA, 22 bp, Artificial Sequence
  Description of Artificial Sequence Synthetic primer
cacccccgacgaagatgaccac

SEQ ID NO: 13 - DNA, 22 bp, Artificial Sequence
  Description of Artificial Sequence Synthetic primer
cggcccgggctccaggtagatg

SEQ ID NO: 14 - DNA, 20 bp, Artificial Sequence
  Description of Artificial Sequence Synthetic primer
ctgacaggcctcaagaaagg

SEQ ID NO: 15 - DNA, 20 bp, Artificial Sequence
  Description of Artificial Sequence Synthetic primer
aacctgttccagacgtggtc

SEQ ID NO: 16 - DNA, 18 bp, Artificial Sequence
  Description of Artificial Sequence Synthetic primer
ggccgtggctctggtctt

SEQ ID NO: 17 - DNA, 21 bp, Artificial Sequence
  Description of Artificial Sequence Synthetic primer
ggttcatcttgctgccatacc

SEQ ID NO: 18 - DNA, 18 bp, Artificial Sequence
  Description of Artificial Sequence Synthetic primer
ctcgaccatcggcactgt

SEQ ID NO: 19 - DNA, 22 bp, Artificial Sequence
  Description of Artificial Sequence Synthetic primer
agtttcttctctggaggggact

SEQ ID NO: 20 - DNA, 24 bp, Artificial Sequence
  Description of Artificial Sequence Synthetic primer
cacacaactggcctctcattaaat

SEQ ID NO: 21 - DNA, 20 bp, Artificial Sequence
  Description of Artificial Sequence Synthetic primer
tgcccccatcaagaaacact

SEQ ID NO: 22 - DNA, 21 bp, Artificial Sequence
  Description of Artificial Sequence Synthetic primer
ttggcgctcaacttttacgaa

SEQ ID NO: 23 - DNA, 19 bp, Artificial Sequence
  Description of Artificial Sequence Synthetic primer
gagcgaatgtccttcccca

SEQ ID NO: 24 - DNA, 20 bp, Artificial Sequence
  Description of Artificial Sequence Synthetic primer
gagccctttccagctctctt

SEQ ID NO: 25 - DNA, 20 bp, Artificial Sequence
  Description of Artificial Sequence Synthetic primer
ccaggtctttctccaccttg

SEQ ID NO: 26 - DNA, 20 bp, Artificial Sequence
  Description of Artificial Sequence Synthetic primer
acaacttcacagaggcccag

SEQ ID NO: 27 - DNA, 20 bp, Artificial Sequence
  Description of Artificial Sequence Synthetic primer
tttcccagagatccctttca

SEQ ID NO: 28 - DNA, 20 bp, Artificial Sequence
  Description of Artificial Sequence Synthetic primer
acggccagaagccagtagtc

SEQ ID NO: 29 - DNA, 25 bp, Artificial Sequence
  Description of Artificial Sequence Synthetic primer
gaccttttgtctgaactccctgtag

SEQ ID NO: 30 - DNA, 23 bp, Artificial Sequence
  Description of Artificial Sequence Synthetic primer
agaacttctgatgtttcttccag

SEQ ID NO: 31 - DNA, 20 bp, Artificial Sequence
  Description of Artificial Sequence Synthetic primer
ctggagacttggctggtata

SEQ ID NO: 32 - DNA, 20 bp, Artificial Sequence
  Description of Artificial Sequence Synthetic primer
aagcaccagcacctgtaccg

SEQ ID NO: 33 - DNA, 22 bp, Artificial Sequence
  Description of Artificial Sequence Synthetic primer
cctttacagtgtccatcctctg

SEQ ID NO: 34 - DNA, 22 bp, Artificial Sequence
  Description of Artificial Sequence Synthetic primer
aagagttcattctcaacagccc

SEQ ID NO: 35 - DNA, 21 bp, Artificial Sequence
  Description of Artificial Sequence Synthetic primer
cttggacagactctgagctgc

SEQ ID NO: 36 - DNA, 21 bp, Artificial Sequence
  Description of Artificial Sequence Synthetic primer
ttaccgaagagttggcgtatg

SEQ ID NO: 37 - DNA, 21 bp, Artificial Sequence
  Description of Artificial Sequence Synthetic primer
atcttctggccgttgataaca

SEQ ID NO: 38 - DNA, 22 bp, Artificial Sequence
  Description of Artificial Sequence Synthetic primer
ataccgcaaagagcacgagaag

SEQ ID NO: 39 - DNA, 25 bp, Artificial Sequence
  Description of Artificial Sequence Synthetic primer
ctcaagagcagcgaaagcgtcacag

SEQ ID NO: 40 - DNA, 22 bp, Artificial Sequence
  Description of Artificial Sequence Synthetic primer
agtcccttgccctttgtacaca

SEQ ID NO: 41 - DNA, 21 bp, Artificial Sequence
  Description of Artificial Sequence Synthetic primer
gatccgagggcctcactaaac

SEQ ID NO: 42 - DNA, 57 bp, Artificial Sequence
  Description of Artificial Sequence Synthetic primer
tagcaaaataactcagatcgccattttctttaacttgcaaaaaatgccatccttctg

SEQ ID NO: 43 - DNA, 57 bp, Artificial Sequence
  Description of Artificial Sequence Synthetic primer
cagaaggatggcattttttgcaagttaaagaaaatggcgatctgagttattttgcta

SEQ ID NO: 44 - DNA, 14121 bp, Homo sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgacgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagaccctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacatttgcacaccgtgacttcagtgcagaatatgaagaagatggcaaatatgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcagtgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 45 - DNA, 7556 bp, Homo sapiens
gcaggcgccttcgcggaccgagcctgacggagccggaggctgggagccgcggcggcctggggaagtgtttggattgtgagctatttcagaactgttctcaggactcattattttaacatttgggagaaacacagccagaagatgcacacttgactgaaggaggacagggaatctgaagactccggatgacatcagagctacttttcaacagccttctcaattttctttctcagaaagcagaggctcagagcttggagacagacgaacactgatatttgcatttaatggggaacaaaagatgaagaaggaaaaggaatatattcactaaggattctatctgcttactgctacagacctatgtgttaaggaattcttctcctcctccttgcgtagaagttgatcagcactgtggtcagactgcatttatcttgtcattgccagaagaaatcttggacagaatgtaacagtacgtctctctctgattgcgatggaaggtgataaactgatactcctttattaaagttacatcgcactcaccacagaaaaccattctttaaagtgaatagaaaccaagcccttgtgaacacttctattgaacatgactcatggagaagagcttggctctgatgtgcaccaggattctattgttttaacttacctagaaggattactaatgcatcaggcagcagggggatcaggtactgccgttgacaaaaagtctgctgggcataatgaagaggatcagaactttaacatttctggcagtgcatttcccacctgtcaaagtaatggtccagttctcaatacacatacatatcaggggtctggcatgctgcacctcaaaaaagccagactgttgcagtcttctgaggactggaatgcagcaaagcggaagaggctgtctgattctatcatgaatttaaacgtaaagaaggaagctttgctagctggcatggttgacagtgtgcctaaaggcaaacaggatagcacattactggcctctttgcttcagtcattcagctctaggctgcagactgttgctctgtcacaacaaatcaggcagagcctcaaggagcaaggatatgccctcagtcatgattctttaaaagtggagaaggatttaaggtgctatggtgttgcatcaagtcacttaaaaactttgttgaagaaaagtaaagttaaagatcaaaagcctgatacgaatcttcctgatgtgactaaaaacctcatcagagataggtttgcagagtctcctcatcatgttggacaaagtggaacaaaggtcatgagtgaaccgttgtcatgtgctgcaagattacaggctgttgcaagcatggtggaaaaaagggctagtcctgccacctcacctaaacctagtgttgcttgtagccagttagcattacttctgtcaagcgaagcccatttgcagcagtattctcgagaacacgctttaaaaacgcaaaatgcaaatcaagcagcaagtgaaagacttgctgctatggccagattgcaagaaaatggccagaaggatgttggcagttaccagctcccaaaaggaatgtcaagccatcttaatggtcaggcaagaacatcatcaagcaaactgatggctagcaaaagtagtgctacagtgtttcaaaatccaatgggtatcattccttcttcccctaaaaatgcaggttataagaactcactggaaagaaacaatataaaacaagctgctaacaatagtttgcttttacatcttcttaaaagccagactatacctaagccaatgaatggacacagtcacagtgagagaggaagcatttttgaggaaagtagtacacctacaactattgatgaatattcagataacaatcctagttttacagatgacagcagtggtgatgaaagttcttattccaactgtgttcccatagacttgtcttgcaaacaccgaactgaaaaatcagaatctgaccaacctgtttccctggataacttcactcaatccttgctaaacacttgggatccaaaagtcccagatgtagatatcaaagaagatcaagatacctcaaagaattctaagctaaactcacaccagaaagtaacacttcttcaattgctacttggccataagaatgaagaaaatgtagaaaaaaacaccagccctcagggagtacacaatgatgtgagcaagttcaatacacaaaattatgcaaggacttctgtgatagaaagccccagtacaaatcggactactccagtgagcactccacctttacttacatcaagcaaagcagggtctcccatcaatctctctcaacactctctggtcatcaaatggaattccccaccatatgtctgcagtactcagtctgaaaagctaacaaatactgcatctaaccactcaatggaccttacaaaaagcaaagacccaccaggagagaaaccagcccaaaatgaaggtgcacagaactctgcaacgtttagtgccagtaagctgttacaaaatttagcacaatgtggaatgcagtcatccatgtcagtggaagagcagagacccagcaaacagctgttaactggaaacacagataaaccgataggtatgattgatagattaaatagccctttgctctcaaataaaacaaatgcagttgaagaaaataaagcatttagtagtcaaccaacaggtcctgaaccagggctttctggttctgaaatagaaaatctgcttgaaagacgtactgtcctccagttgctcctggggaaccccaacaaagggaagagtgaaaaaaaagagaaaactcccttaagagatgaaagtactcaggaacactcagagagagctttaagtgaacaaatactgatggtgaaaataaaatctgagccttgtgatgacttacaaattcctaacacaaatgtgcacttgagccatgatgctaagagtgccccattcttgggtatggctcctgctgtgcagagaagcgcacctgccttaccagtgtccgaagactttaaatcggagcctgtttcacctcaggatttttctttctccaagaatggtctgctaagtcgattgctaagacaaaatcaagatagttacctggcagatgattcagacaggagtcacagaaataatgaaatggcacttctagaatcaaagaatctttgcatggtccctaagaaaaggaagctttatactgagccattagaaaatccatttaaaaagatgaaaaacaacattgttgatgctgcaaacaatcacagtgccccagaagtactgtatgggtccttgcttaaccaggaagagctgaaatttagcagaaatgatcttgaatttaaatatcctgctggtcatggctcagccagcgaaagtgaacacaggagttgggccagagagagcaaaagctttaatgttctgaaacagctgcttctctcagaaaactgtgtgcgagatttgtccccgcacagaagtaactctgtggctgacagtaaaaagaaaggacacaaaaataatgtgaccaacagcaaacctgaatttagcatttcttctttaaatggactgatgtacagttccactcagcccagcagttgcatggataacaggacattttcatacccaggtgtagtaaaaactcctgtgagtcctactttccctgagcacttgggctgtgcagggtctagaccagaatctgggcttttgaatgggtgttccatgcccagtgagaaaggacccattaagtgggttatcactgatgcggagaagaatgagtatgaaaaagactctccaagattgaccaaaaccaacccaatactatattacatgcttcaaaaaggaggcaattctgttaccagtcgagaaacacaagacaaggacatttggagggaggcttcatctgctgaaagtgtctcacaggtcacagccaaagaagagttacttcctactgcagaaacgaaagcttctttctttaatttaagaagcccttacaatagccatatgggaaataatgcttctcgcccacacagcgcaaatggagaagtttatggacttctgggaagcgtgctaacgataaagaaagaatcagaataaaatgtacctgccatccagttttggatctttttaaaactaatgagtatgaacttgagatctgtataaataagagcatgatttgaaaaaaagcatggtataattgaaacttttttcattttgaaaagtattggttactggtgatgttgaaatatgcatactaatttttgcttaacattagatgtcatgaggaaactactgaactagcaattggttgtttaacacttctgtatgcatcagataacaactgtgagtagcctatgaatgaaattcttttataaatattaggcataaattaaaatgtaaaactccattcatagtggattaatgcattttgctgcctttattagggtactttattttgcttttcagaagtcagcctacataacacatttttaaagtctaaactgttaaacaactctttaaaggataattatccaataaaaaaaaacctagtgctgattcacagcttattatccaattcaaaaataaattagaaaaatatatgcttacatttttcacttttgctaaaaagaaaaaaaaaaggtgtttatttttaactcttggaagaggttttgtggttcccaatgtgtctgtcccaccctgatccttttcaatatatatttctttaaaccttgtgctacttagtaaaaattgattacaattgagggaagtttgatagatcctttaaaaaaaaggcagatttccattttttgtattttaactactttactaaattaatactcctccttttacagaattagaaaagttaacatttatctttaggtggtttcctgaaaagttgaatatttaagaaattgtttttaacagaagcaaaatggcttttctttggacagttttcaccatctcttgtaaaagttaattctcaccattcctgtggtacctgcgagtgttatgaccaggattccttaaacctgaactcagaccacttgcattagaaccatctggagcacttgttttaaaatgcagattcataggcagcatctcagatctacagaacaagaatctctgctaagtggacctggaatcttccatctgcatcttaacatgctctctaggtgtttcttgtgtttgagaaccatgacttatgactttcctcagaacatgagactgtaaaacaaaaacaaaaaactatgtgatgcctctattttccccaatacagtcacacatcagctcaaaatttgcaatattgtagttcatatattaccgttatgtctttggaaatcgggttcagaacactttttatgacaaaaattgggtggaggggataactttcatatctggctcaacatctcaggaaaatctgtgattatttgtgtgttctaatgagtaacatctacttagttagccttagggatggaaaaacagggccacttaccaaactcaggtgattccaggatggtttggaaacttctcctgaatgcatccttaacctttattaaaaccattgtcctaagaacaatgccaacaaagcttacaacatttagtttaaacccaagaagggcactaaactcagattgactaaataaaaagtacaaagggcacatatacgtgacagaattgtacacaatcactccattggatcttttactttaaagtagtgatgaaaagtacatgttgatactgtcttagaagaaattaatatattagtgaagccacatggggtttcagttgcgaaacaggtctgtttttatgttcagtttgtacaatccacaattcattcaccagatattttgttcttaattgtgaaccaggttagcaaatgacctatcaaaaattattctataatcactactagttaggatattgatttaaaattgttctacttgaagtggtttctaagatttttatattaaaaataggtgtgatttcctaatatgatctaaaaccctaaatggttatttttcctcagaatgatttgtaaatagctactggaaatattatacagtaataggagtgggtattatgcaacatcatggagaagtgaaggcataggcttattctgacataaaattccactggccagttgaatatattctattccatgtccatactatgacaatcttattgtcaacactatataaataagcttttaaacaagtcatttttcttgatcgttgtggaaggtttggagccttagaggtatgtcagaaaaaatatgttggtattctcccttgggtagggggaaatgacctttttacaagagagtgaaatttaggtcagggaaaagaccaagggccagcattgctacttttgtgtgtgtgtgtgtgggttttgttttgtttttttggttggctggttgttttcgttgttgttaacaaaggaatgagaatatgtaatacttaaataaacatgaccacgaagaatgctgttctgatttactagagaatgttcccaatttgaatttagggtgattttaaagaacagtgagaaagggcatacatccacagattcactttgtttatgcatatgtagatacaaggatgcacatatacacattttcaaggactattttagatatctagacaatttcttctaataaagtcatttgtgaaagggtactacagcttattgacatcagtaaggtagcattcattacctgtttattctctgctgcatcttacagaagagtaaactggtgagagtatatattttatatatatatatatatatatatatataatatgtatatatatatatattgacttgttacatgaagatgttaaaatcggtttttaaaggtgatgtaaatagtgatttccttaatgaaaaatacatattttgtattgttctaatgcaacagaaaagccttttaatctctttggttcctgtatattccatgtataagtgtaaatataatcagacaggtttaaaagttgtgcatgtatgtatacagttgcaagtctggacaaatgtatagaataaaccttttatttaagttgtgattacctgctgcatgaaaagtgcatgggggaccctgtgcatctgtgcatttggcaaaatgtcttaacaaatcagatcagatgttcatcctaacatgacagtattccatttctggacatgacgtctgtggtttaagctttgtgaaagaatgtgctttgattcgaagggtcttaaagaatttttttaatcgtcaaccacttttaaacataaagaattcacacaactactttcatgaattttttaatcccattgcaaacattattccaagagtatcccagtattagcaatactggaatataggcacattaccattcatagtaagaattctggtgtttacacaaccaaatttgatgcgatctgctcagtaatataatttgccatttttattagaaatttaatttcttcatgtgatgtcatgaaactgtacatactgcagtgtgaatttttttgttttgttttttaatcttttagtgtttacttcctgcagtgaatttgaataaatgagaaaaaatgcattgtc

SEQ ID NO: 46 - DNA, 7831 bp, Homo sapiens
tgcgctttccacctaccagaccctgaaagaaagtgtcaggagccggtgcaaaacccagtttaagttcaagaagacatttgcaagtgcaagaggccaagcagtttgaagaagtgtaagagattttttttccttcgaaagaatatatttttaaagaaaccagccagtccgcggaaagcaacagcagttttttttttttttgcctctttttcttattttagatcgagaggtttttcttgcttttcttcccttttttttctttttgcaaacaaaacaaaaaacagcatagaagaaagagcaaaataaagaagaagaagaggaggaagagagggaaagagaggaagggaaaaaaaacaccaacccgggcagaggaggaggtgcggcggcggcggcggcggcggcagcggcggcagcggcgcggcggcggctcggaccccctcccccggctccccccatcagtgcagctctccgggcgatgccagaatagatgccggggcaatgtcccgccgcaaacagggcaacccgcagcacttgtcccagagggagctcatcaccccagaggctgaccatgtggaggccgccatcctcgaagaagacgagggtctggagatagaggagccaagtggcctggggctgatggtgggtggccccgaccctgacctgctcacctgtggccagtgtcaaatgaacttccccttgggggacatcctggtttttatagagcacaaaaggaagcagtgtggcggcagcttgggtgcctgctatgacaaggccctggacaaggacagcccgccaccctcctcacgctccgagctcaggaaagtgtccgagccggtggagatcgggatccaagtcacccccgacgaagatgaccacctgctctcacccacgaaaggcatctgtcccaagcaggagaacattgcaggtaaagatgagccttccagctacatttgcacaacatgcaagcagcccttcaacagcgcgtggttcctgctgcagcacgcgcagaacacgcacggcttccgcatctacctggagcccgggccggccagcagctcgctcacgccgcggctcaccatcccgccgccgctcgggccggaggccgtggcgcagtccccgctcatgaatttcctgggcgacagcaaccccttcaacctgctgcgcatgacgggccccatcctgcgggaccacccgggcttcggcgagggccgcctgccgggcacgccgcctctcttcagtcccccgccgcgccaccacctggacccgcaccgcctcagtgccgaggagatggggctcgtcgcccagcaccccagtgccttcgaccgagtcatgcgcctgaaccccatggccatcgactcgcccgccatggacttctcgcggcggctccgcgagctggcgggcaacagctccacgccgccgcccgtgtccccgggccgcggcaaccctatgcaccggctcctgaaccccttccagcccagccccaagtccccgttcctgagcacgccgccgctgccgcccatgccccctggcggcacgccgcccccgcagccgccagccaagagcaagtcgtgcgagttctgcggcaagaccttcaagttccagagcaatctcatcgtgcaccggcgcagtcacacgggcgagaagccctacaagtgccagctgtgcgaccacgcgtgctcgcaggccagcaagctcaagcgccacatgaagacgcacatgcacaaggccggctcgctggccggccgctccgacgacgggctctcggccgccagctcccccgagcccggcaccagcgagctggcgggcgagggcctcaaggcggccgacggtgacttccgccaccacgagagcgacccgtcgctgggccacgagccggaggaggaggacgaggaggaggaggaggaggaggaggagctgctactggagaacgagagccggcccgagtcgagcttcagcatggactcggagctgagccgcaaccgcgagaacggcggtggtggggtgcccggggtcccgggcgcggggggcggcgcggccaaggcgctggctgacgagaaggcgctggtgctgggcaaggtcatggagaacgtgggcctaggcgcactgccgcagtacggcgagctcctggccgacaagcagaagcgcggcgccttcctgaagcgtgcggcgggcggcggggacgcgggcgacgacgacgacgcgggcggctgcggggacgcgggcgcgggcggcgcggtcaacgggcgcgggggcggcttcgcgccaggcaccgagcccttccccgggctcttcccgcgcaagcccgcgccgctgcccagccccgggctcaacagcgccgccaagcgcatcaaggtggagaaggacctggagctgccgcccgccgcgctcatcccgtccgagaacgtgtactcgcagtggctggtgggctacgcggcgtcgcggcacttcatgaaggaccccttcctgggcttcacggacgcacgacagtcgcccttcgccacgtcgtccgagcactcgtccgagaacggcagcctgcgcttctccacgccgcccggggacctgctggacggcggcctctcgggccgcagcggcacggccagcggaggcagcaccccgcacctgggcggcccgggccccgggcggcccagctccaaggagggccgccgcagcgacacgtgcgagtactgcggcaaggtgttcaagaactgcagcaacttgacggtgcaccggcggagccacaccggcgagcggccttacaagtgcgagctgtgcaactacgcgtgcgcgcagagcagcaagctcacgcgccacatgaagacgcacgggcagatcggcaaggaggtgtaccgctgcgacatctgccagatgcccttcagcgtctacagcaccctggagaaacacatgaaaaagtggcacggcgagcacttgctgactaacgacgtcaaaatcgagcaggccgagaggagctaagcgcgcgggccccggcgccccgcacctgtacagtggaaccgttgccaaccgagagaatgctgacctgacttgcctccgtgtcaccgccaccccgcaccccgcgtgtccccggggcccaggggaggcggcactccaacctaacctgtgtctgcgaagtcctatggaaacccgagggttgattaaggcagtacaaattgtggagccttttaactgtgcaataatttctgtatttattgggttttgtaatttttttggcatgtgcaggtactttttattattattttttctgtttgaattcctttaagagattttgttgggtatccatcccttctttgttttttttttaacccggtagtagcctgagcaatgactcgcaagcaatgttagaggggaagcatatcttttaaattataatttggggggaggggtggtgctgcttttttgaaatttaagctaagcatgtgtaatttcttgtgaagaagccaacactcaaatgacttttaaagttgtttactttttcattccttccttttttttgtcctgaaataaaaagtggcatgcagttttttttttaattattttttaattttttttttggtttttgtttttggggtggggggtgtggatgtacagcggataacaatctttcaagtcgtagcactttgtttcagaactggaatggagatgtagcactcatgtcgtcccgagtcaagcggccttttctgtgttgatttcggctttcatattacataagggaaaccttgagtggtggtgctgggggaggcaccccacagactcagcgccgccagagatagggtttttggagggctcctctgggaaatggcccgacagcattctgaggttgtgcatgaccagcagatactatcctgttggtgtgccctggggtgccatggctgctattcgctgtagattaggctacataaaatgggctgagggtacctttttggggagatggggtggcctgcagtgacacagaaaggaagaaactagcggtgttcttttaggcgttttctggcttgacggcttctctctttttttaaatcacccccaccacataaatctcaaatcctatgttgctacaaggggtcatccatcatttcccaagcagacgaatgccctaattaattgaagttagtgttctctcatttaatgcacactgatgatattgtagggatgggtggggtggggatcttgcaaatttctattctcttttactgaaaaagcaggggatgagttccatcagaaggtgcccagcgctacttcccaggtttttattttttttttcctatctcattaggttggaaggtactaaatattgaactgttaagattagacatttgaattctgttgacccgcactttaaagcttttgtttgcatttaaattaaatggcttctaaacaagaaattgcagcatattcttctctttggcccagaggtgggttaaactgtaagggacagctgagattgagtgtcagtattgctaagcgtggcattcacaatactggcactataaagaacaaaataaaataataatttataggacagtttttctactgccattcaatttgatgtgagtgccttgaaaactgatcttcctatttgagtctcttgagacaaatgcaaaactttttttttgaaatgaaaagactttttaaaaaagtaaaacaagaaaagtacattctttagaaactaacaaagccacatttactttaagtaaaaaaaaaaaaaattctggttgaagatagaggatatgaaatgccataagacccaatcaaatgaagaaataaacccagcacaaccttggacatccattagctgaattatcctcagccccttttgtttttgggacaacgctgcttagatatggagtggaggtgatttactgctgaattaaaactcaagtgacacaagttacaagttgatatcgttgaatgaaaagcaaaacaaaaacaattcaggaacaacggctaattttttctaaagttaaatttagtgcactctgtcttaaaaatacgtttacagtattgggtacatacaagggtaaaaaaaaaattgtgtgtatgtgtgttggagcgatctttttttttcaaagtttgcttaataggttatacaaaaatgccacagtggccgcgtgtatattgttttcttttggtgacggggttttagtatatattatatatattaaaatttcttgattactgtaaaagtggaccagtatttgtaataatcgagaatgcctgggcattttacaaaacaagaaaaaaaatacccttttcttttccttgaaaatgttgcagtaaaatttaaatggtgggtctataaatttgttcttgttacagtaactgtaaagtcggagttttagtaaatttttttctgccttgggtgttgaatttttatttcaaaaaaaatgtatagaaacttgtatttggggattcaaaggggattgctacaccatgtagaaaaagtatgtagaaaaaaagtgcttaatattgttattgctttgcagaaaaaaaaaaaatcacatttctgacctgtacttatttttctcttcccgcctccctctggaatggatatattggttggttcatatgatgtaggcacttgctgtatttttactggagctcgtaattttttaactgtaagcttgtccttttaaagggatttaatgtacctttttgttagtgaatttggaaataaaaagaaaaaaaaaacaaaaacaaacaggctgccataatatatttttttaatttggcaggataaaatattgcaaaaaaaacacatttgtatgttaagtcctattgtacaggagaaaaagggttgtttgacaacctttgagaaaaagaaacaaaaggaagtagttaaatgctttggttcacaaatcatttagttgtatatattttttgtcggaattggcctacacagagaaccgttcgtgttgggcttctctctgaacgccccgaaccttgcatcaaggctccttggtgtggccacagcagaccagatgggaaattatttgtgttgagtggaaaaaaatcagtttttgtaaagatgtcagtaacattccacatcgtcctccctttctctaagaggccatctctaagatgtcagatgtagaggagagagagcgagagaacatcttccttctctaccatcactcctgtggcggtcaccaccaccacctctcccgcccttaccagcagaaagcaatgcaaactgagctgctttagtccttgagaaattgtgaaacaaacacaaatatcataaaaggagctggtgattcagctgggtccaggtgaagtgacctgctgttgagaccggtacaaattggatttcaggaaggagactccatcacagccaggacctttcgtgccatggagagtgttggcctcttgtctttcttccctgctttgctgctttgctctctgaaacctacattccgtcagtttccgaatgcgagggcctgggatgaatttggtgcctttccatatctcgttctctctccttcccctgcgtttcctctccatccttcatcctccattggtcctttttttttctttcattttttatttaatttcttttcttcctgtctgttcctcccctaatcctctattttatttttattttttgtaaagccaagtagctttaagataaagtggtggtcttttggatgagggaataatgcatttttaaataaaataccaatatcaggaagccattttttatttcaggaaatgtaagaaaccattatttcaggttatgaaagtataaccaagcatccttttgggcaattccttaccaaatgcagaagcttttctgttcgatgcactctttcctccttgccacttacctttgcaaagttaaaaaaaaggggggagggaatgggagagaaagctgagatttcagtttcctactgcagtttcctacctgcagatccaggggctgctgttgcctttggatgccccactgaggtcctagagtgcctccagggtggtcttcctgtagtcataacagctagccagtgctcaccagcttaccagattgccaggactaagccatcccaaagcacaagcattgtgtgtctctgtgactgcagagaagagagaattttgcttctgttttgtgtttaaaaaaccaacacggaagcagatgatcccgagagagaggcctctagcatgggtgacccagccgacctcaggccggtttccgcactgccacaactttgttcaaagttgcccccaattggaacctgccacttggcattagagggtctttcatggggagagaaggagactgaattactctaagcaaaatgtgaaaagtaaggaaatcagcctttcatcccggtcctaagtaaccgtcagccgaaggtctcgtggaacacaggcaaacccgtgattttggtgctccttgtaactcagccctgcaaagcaaagtcccattgatttaagttgtttgcatttgtactggcaaggcaaaatatttttattaccttttctattacttattgtatgagcttttgttgtttacttggaggttttgtcttttactacaagtttggaactatttattattgcttggtatttgtgctctgtttaagaaacaggcacttttttttattatggataaaatgttgagatgacaggaggtcatttcaatatggcttagtaaaatatttattgttcctttattctctgtacaagattttgggcctctttttttccttaatgtcacaatgttgagttcagcatgtgtctgccatttcatttgtacgcttgttcaaaaccaagtttgttctggtttcaagttataaaaataaattggacatttaacttgatctccaaa

SEQ ID NO: 47 - RNA, 22 nt, Homo sapiens
gcaaguuaaagaaaaucaggau

SEQ ID NO: 48 - DNA, 22 bp, Homo sapiens
gcaagttaaagaaaatggcgat

SEQ ID NO: 49 - RNA, 21 nt, Homo sapiens
uucccugagacuuuucaggag

SEQ ID NO: 50 - RNA, 21 nt, Mus sp.
ugcccagagacuucucaggag